(19) United States
(12) Patent Application Publication
(10) Pub. No.: US 2023/0329583 A1
(43) Pub. Date: Oct. 19, 2023

(54) DETECTING AND PREVENTING SLEEPWALKING EVENTS

(71) Applicant: Sleep Number Corporation, Minneapolis, MN (US)

(72) Inventors: Gary N. Garcia Molina, Madison, WI (US); Yehor Shcherbakov, Kyiv (UA)

(21) Appl. No.: 18/131,218

(22) Filed: Apr. 5, 2023

Related U.S. Application Data
(60) Provisional application No. 63/330,442, filed on Apr. 13, 2022.

(51) Int. Cl. A61B 5/11; A61B 5/00; A47C 21/04

(52) U.S. Cl. CPC A61B 5/1115; A61B 5/4809; A61B 5/7264; A61B 5/7275; A47C 21/048

(57) ABSTRACT

A bed system detects and prevents user sleepwalking events. The system includes a sensor and computer system to receive data from the sensor during the user's sleep session, provide, as input to a sleep state classifier, a first portion of the data, the sleep state classifier using a machine learning model to determine the user's sleep states, receive, as output, a sleep state classification for the user, provide, as input to a bed exit detection classifier, a second portion of the sensor data, the bed exit detection classifier using a machine learning model to determine when the user exits the bed, receive, as output, a bed exit detection classification for the user, determine whether (i) the sleep state classification satisfies a first threshold condition and (ii) the bed exit detection classification satisfies a second threshold condition, generate, based on the determination, a probability of a sleepwalking event for the user.

### INCORPORATION BY REFERENCE

This application claims priority to U.S. Provisional Application Serial No. 63/330,442, filed on Apr. 13, 2022, the disclosure of which is incorporated by reference in its entirety.

### TECHNICAL FIELD

The present document relates to detecting and preventing sleepwalking events of a user in a bed.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

This disclosure generally relates to systems, methods, and techniques for detecting sleepwalking events of a user of a bed and preventing sleepwalking events (e.g., somnambulism) of that user during sleep sessions. The disclosed technology can be performed with a bed system having smart sensors. The disclosed technology can also be performed with any other type of sleep system, including but not limited to wearable devices. Sensors, such as temperature and/or pressure sensors of a bed system or other sensors (e.g., wearable sensors/devices), can be used to collect user data. Machine learning techniques can be applied to the collected user data to determine sleep state classification and bed presence detection of the user during a sleep session. Using the sleep state classification and bed presence detection, a computing system can detect a sleepwalking event. The computing system can also generate output about the detected sleepwalking event. The output can be provided to the user and/or healthcare providers of the user. The computing system can also determine a probability that the user will sleepwalk during a sleep session (e.g., current, subsequent, or future sleep sessions), which can be based on historic detections of sleepwalking events for that user. Based on the probability of sleepwalking onset, the computing system can generate one or more outputs, such as interventions, to prevent the user from sleepwalking during the sleep session. By intervening and preventing the onset of the sleepwalking event, the user can continue to experience uninterrupted sleep, thereby improving the user's overall sleep experience and/or sleep quality.

Sleepwalking, or somnambulism, can occur within 1 to 3 hours of falling asleep (e.g., 1 to 2 sleep cycles), when non-rapid-eye-movement (NREM) sleep is most prevalent. NREM includes N1, N2, and N3 sleep, and the latter is when sleep is the deepest. Each episode may last from approximately 30 seconds to 30 minutes. Sleepwalking can occur in NREM phases, usually in N3, or stage 3. Moreover, since sleepwalking occurs during a specific stage of a user's sleep cycle, sleepwalking may typically occur around the same time during each sleep session (e.g., each night). Therefore, the disclosed technology can be used to detect when the user experiences sleepwalking events during prior sleep sessions to then detect onset of a sleepwalking event during a given sleep session (e.g., current) and perform an intervention to prevent the onset of the sleepwalking event.

The intervention can be performed to make deep sleep occur at a different timing (preferably earlier) compared to prior sleep walking episodes in the given sleep session. The intervention can also be performed to make deep sleep deeper (e.g., by increasing power in the 0.5 to 4 Hz band in a sleep EEG) during the given sleep session. The intervention can include adjusting a microclimate of the user's bed. For example, the microclimate of the bed can be increased in temperature by a threshold amount (e.g., in an order of 1° C.) to promote a faster decrease in core body temperature (CBT), which can shorten latency to N3 sleep and thus prevent onset of the sleepwalking event. As another example, the intervention can include delivering sensory stimulation to the user that shallows sleep such that N3 sleep occurs later, thereby preventing onset of the sleepwalking event.

As described throughout this disclosure, non-invasive, contact-free, real-time sleep state and bed exit detection can be performed in order to detect sleepwalking events and prevent the onset of sleepwalking events to improve the user's sleep quality without disturbing the user during their sleep session. One or more machine learning models can be used to train a classifier to determine sleep staging of the user based on ballistocardiography (BCG) signals from that user. In some implementations, sensors of the bed system can be used to detect the BCG signals and one or more other signals that can be used to determine the user's sleep state. The machine learning models can be trained using robust training datasets that include BCG signals tagged with different sleep states. The training BCG signals can be received from any variety of sources, including but not limited to a data store, cloud service, database, and/or beds (e.g., smart beds or other bed systems having sensors). Moreover, to detect bed exits, a pump or other device may include a sensor that monitors pressure of one or more air bladders/chambers in a mattress of the bed system. When the user enters the bed system, the pressure may quickly increase. One or more techniques can be performed to filter the noise and other influences on the increased pressure in order to generate a determination of user occupancy and/or user exit from the bed system. A combination of the sleep state classification and bed exit detection can then be used to detect a sleepwalking event of the user, as described herein.

Some embodiments described herein include a system for detecting sleepwalking events of a user in a bed, the system including: at least one sensor and a computer system in communication with the at least one sensor, the computer system being able to: receive sensor data from the at least one sensor during a sleep session of a user of a bed, provide, as input to a sleep state classifier, a first portion of the sensor data, the sleep state classifier using a machine-learning model to determine the user's sleep states during the sleep session, receive, as output from the sleep state classifier, a sleep state classification for the user, provide, as input to a bed exit detection classifier, a second portion of the sensor data, the bed exit detection classifier using a machine-learning model to determine when the user exits the bed during the sleep session, receive, as output from the bed exit detection classifier, a bed exit detection classification for the user, determine whether (i) the sleep state classification for the user satisfies a first threshold condition and (ii) the bed exit detection classification for the user satisfies a second threshold condition, identify, based on a determination that the first and the second threshold conditions are satisfied, a sleepwalking event for the user, and generate output based on identification of the sleepwalking event.

Embodiments described herein can include one or more optional features. For example, the first threshold condition can be a N3 sleep state. The second threshold condition can be detection of at least one bed exit event. The computer system can also: identify, based on (i) the determination that the first and the second threshold conditions are satisfied and (ii) a total time from a start of the sleep session is within a threshold time range since sleep onset, wherein the threshold time range is associated with prior sleep session time ranges associated the user, the sleepwalking event for the user. The threshold range can be 1 to 3 hours. The threshold range can also be 1 to 2 sleep cycles. Generating the output can include storing the sleep state classification, the bed exit detection classification, and the total time in a data store. Generating the output can include storing the identification of the sleepwalking event in a data store. The identification of the sleepwalking event can include information about a time during the user's sleep session when the sleepwalking event was identified.

As another example, generating the output can include generating a notification indicating that the sleepwalking event was identified during the user's sleep session. The computer system can also transmit the notification to a user device of the user for presentation in a graphical user interface (GUI) display when the user wakes up from the sleep session. The computer system may transmit the notification to a user device of a healthcare provider associated with the user, the notification being a machine-instruction to engage an automated device.

Some embodiments described herein include a system for detecting and preventing sleepwalking events of a user in a bed, the system including: at least one sensor and a computer system in communication with the at least one sensor, the computer system being configured to: receive sensor data from the at least one sensor during a sleep session of a user of a bed, provide, as input to a sleep state classifier, a first portion of the sensor data, the sleep state classifier using a machine learning model to determine the user's sleep states during the sleep session, receive, as output from the sleep state classifier, a sleep state classification for the user, provide, as input to a bed exit detection classifier, a second portion of the sensor data, the bed exit detection classifier using a machine learning model to determine when the user exits the bed during the sleep session, receive, as output from the bed exit detection classifier, a bed exit detection classification for the user, determine whether (i) the sleep state classification for the user satisfies a first threshold condition and (ii) the bed exit detection classification for the user satisfies a second threshold condition, generate, based on a determination that the first and the second threshold conditions are satisfied, a probability of a sleepwalking event for the user, and generate output based on the probability of the sleepwalking event for the user.

Embodiments described herein can include one or more optional features. For example, the probability of the sleepwalking event can indicate a likelihood that the user will experience the sleepwalking event within a threshold amount of time from a current time during the sleep session. The threshold amount of time can be 1 to 15 minutes. The first threshold condition can be a N3 sleep state. In some implementations, the bed exit detection classification can indicate a time at which bed presence of the user is detected, and the bed exit detection classification for the user satisfies the second threshold condition based on the time at which the bed presence of the user is detecting corresponding to historic data of detected sleepwalking events for the user.

As another example, generating output based on the probability of the sleepwalking event for the user can include generating an intervention to prevent onset of the sleepwalking event for the user. The computer system can generate the intervention based on transmitting instructions to a controller of the bed that, when executed by the controller, cause the controller to actuate a heating element in the bed to increase a temperature of a microclimate of the bed by a threshold amount. The threshold amount can be 0.5 to 1° C. The temperature of the microclimate can be increased to approximately to 36° C. As another example, the computer system can generate the intervention based on transmitting instructions to a controller of the bed that, when executed by the controller, cause the controller to generate and output sensory stimulation to the user. The sensory stimulation can include audio, the audio being the user's name. The sensory stimulation can also include vibrations. Sometimes, the computer system can generate the intervention based on transmitting instructions to a controller of the bed that, when executed by the controller, cause the controller to lower an adjustable foundation of the bed to a threshold height.

Embodiments described herein can include a system for preventing sleepwalking events of a user in a bed, the system including a bed having at least one sensor and a computer system in communication with the bed, the computer system being configured to: retrieve, from a data store, historic data for the user, the historic data including information about prior detected sleepwalking events of the user, receive sensor data from the at least one sensor throughout a sleep session of a user of the bed, determine, based on the sensor data, a real-time sleep state classification of the user during the sleep session, determine, based on the sensor data, bed presence detection of the user during the sleep session, determine a probability of onset of a sleepwalking event of the user based on a determination that (i) the real-time sleep state classification is N3 and (ii) the bed presence detection corresponds to the information about prior detected sleepwalking events of the user, and generate output to prevent the onset of the sleepwalking event of the user based on the probability satisfying a threshold condition.

Embodiments described herein can include one or more optional features. For example, the information about prior detected sleepwalking events of the user can include, for each of the prior detected sleepwalking events, a time at which the sleepwalking event was detected during a sleep session of the user. The computer system can also generate and perform the output within 15 minutes of determining that the probability satisfies the threshold condition. The computer system can also determine that the bed presence detection corresponds to the information about prior detected sleepwalking events of the user based on the bed presence detection indicating an in-bed time of the user that is similar to in-bed times associated with the prior detected sleepwalking events of the user. The computer system can generate and perform the intervention based on generating and transmitting instructions to a controller of the bed that, when executed by the controller, cause the controller to actuate a heating element in the bed to increase a temperature of a microclimate of the bed by a threshold amount. The threshold condition can be a likelihood that the user will sleepwalk within 15 minutes of determining (i) and (ii). Sometimes, the computer system can generate and perform the intervention based on generating and transmitting instructions to a controller of the bed that, when executed by the controller, cause the controller to lower an adjustable foundation of the bed to a threshold height.

Embodiments described herein can include a sleep system for determining when a user is sleepwalking, the system including: one or more sensors, at least one output device, one or more processors, and a computer-readable storage medium coupled to the one or more processors having instructions stored thereon that, when executed by the one or more processors, cause the one or more processors to perform operations that include: determining a sleep state of a user based on sensed data received from the one or more sensors during a sleep session of the user, determining a bed presence of the user based on the sensed data received from the one or more sensors during the sleep session of the user, determining whether the user is sleepwalking as a function of (i) determining the sleep state and (ii) determining the bed presence of the user, and generating an output to be presented at the at least one output device and based on a determination that the user is sleepwalking.

Embodiments described herein can include one or more optional features. For example, the operations can also include determining a duration of the sleep session of the user based on the sensed data received from the one or more sensors during the sleep session of the user. Determining whether the user is sleepwalking can be determined further as a function of determining the duration of the sleep session of the user. The operations further can include: storing the output in a data store, the output including an indication that the user is sleepwalking and a time during the sleep session at which the user is sleepwalking. The output can include information about a time during the sleep session at which the user is sleepwalking. The output can also include a notification that the user was sleepwalking during the sleep session.

Embodiments described herein can include a sleep system for preventing onset of a sleepwalking event of a user, the system including: one or more sensors, one or more processors, and a computer-readable storage medium coupled to the one or more processors having instructions stored thereon that, when executed by the one or more processors, cause the one or more processors to perform operations including: retrieving historic sleep data about a user, the historic sleep data including information about prior detected sleepwalking events of the user during other sleep sessions, determining a current sleep state of the user based on sensed data received from the one or more sensors during a sleep session of the user, determining a current bed presence of the user based on the sensed data received from the one or more sensors during the sleep session of the user, generating a probability of a sleepwalking event onset based on whether the current sleep state and the current bed presence of the user correspond to the historic sleep data, and generating output based on the probability of the sleepwalking event onset satisfying a threshold condition.

Embodiments described herein include can include one or more of the following features. For example, performing the intervention by generating and transmitting instructions to a controller of a bed of the user that, when executed by the controller, cause the controller to actuate a heating element in the bed to increase a temperature of a microclimate of the bed.

Embodiments described herein can include a sleep system that can be configured to determine that a user is sleepwalking upon detection of a user exiting a bed during a particular sleep state. Embodiments described herein include one or more of the following features. For example, the detection of the user exiting the bed during the particular sleep state can be within a threshold duration since beginning a sleep session. The threshold duration can be a time period of 3 hours of less. The threshold duration can be a number of sleep cycles of 2 or fewer. The particular sleep state can be N3.

Embodiments described herein include a sleep system that can be configured to prevent a user from sleepwalking upon detection of user activity in a bed that corresponds to historic sleepwalking data for the user. Embodiments described herein can include one or more of the following features. For example, the sleep system can prevent the user from sleepwalking within a threshold amount of time of detecting the user activity in the bed. The threshold amount of time can be 1 to 15 minutes. The sleep system can prevent the user from sleepwalking based on increasing a temperature of a microclimate in the bed. The user activity can include bed presence detection. The user activity can include (i) a particular sleep state and (ii) a bed presence detection. The particular sleep state can be N3.

The devices, system, and techniques described herein may provide one or more of the following advantages. For example, the disclosed technology provides for non-invasively and non-obtrusively monitoring a user's sleep behavior to accurately detect sleepwalking events, determine likelihoods of sleepwalking onset for that user during a given sleep session, and perform an intervention to prevent the somnambulism onset. This contact-free and real-time approach to detecting and preventing sleepwalking can provide more insight into the user's health and improve the user's overall sleep quality.

The disclosed technology can leverage a combination of different metrics to accurately detect and prevent sleepwalking events of the user. For example, the disclosed technology can leverage a combination of sleep state determinations and bed exit analysis to accurately detect if the user sleepwalks and when the user is likely to sleepwalk during a sleep session. The disclosed technology can also leverage historic detections and learnings about the user's sleepwalking routine as well as real-time monitoring of when the user goes to sleep and enters different sleep states during a given sleep session to accurately detect and prevent sleepwalking events. Combining various data points that have been collected and/or determined over time as well as during runtime can provide for accurate detection and prevention of sleepwalking events to improve overall user sleep quality.

Similarly, the disclosed technology provides for detecting and preventing sleepwalking events without the use of additional sensors and/or technology. In other words, existing systems, such as a wearable device and/or a smart bed system, can be used with the disclosed technology in order to accurately detect and prevent somnambulism. The user may not have to purchase, install, or otherwise utilize additional sensors, systems, or devices, to receive information about their sleepwalking and experience improved sleep from the prevention of sleepwalking onset.

As another example, the disclosed technology can provide for overall improvement in operation of a computer or computing system. The disclosed techniques can utilize less processing power and computational resources. Real-time machine learning modeling can be relatively light compared to other models, thereby gaining advantages in terms of memory requirements for hardware and low latency in computation. As a result, sleep state determinations can be made quicker, in real-time, as well as more accurately to then determine quickly and efficiently whether the user is sleepwalking and/or likely to sleepwalk. Alternative methods, on the other hand, may require more processing power, may lag in making such sleep-state determinations, and therefore may not be as accurate in determining current sleep states of the user in real-time to then accurately determine whether the user is sleepwalking and/or likely to sleepwalk.

The disclosed technology can also provide for accurate, no-contact monitoring of the user as the user sleeps. The user may not be required to wear sensors, such as wearable devices, straps, masks, or other sensor signals. The user can merely go to bed and their sleep states and bed presence can be monitored and tracked based on BCG signals or other signals that are sensed by components of the bed (e.g., one or more sensors, sensor pads, sensor strips, sensor arrays, etc.).

Moreover, non-invasive, contactless and real-time monitoring of the user can provide timely information for applications like closed-loop temperature control, comfort adjustment and smart alarm, which can help enhance the sleep quality and sleep efficiency by preventing onset of sleepwalking events. Other methods, on the other hand, may require the user to wear sensors or other contact-based monitoring devices to ensure some level of accuracy of sleep monitoring. Such contact-based monitoring may interfere with sleep comfort, thereby defeating the purpose of improving sleep comfort. Moreover, the contact-based monitoring devices may require more processing power than the disclosed technology. The more processing power, the slower the other methods may be in determining whether the user is sleepwalking and/or likely to sleepwalk during a sleep session. Thus, the other methods may not generate accurate determinations about sleepwalking events. The disclosed technology, on the other hand, can provide for accurate, real-time sleep monitoring while ensuring that the user can continue to experience quality sleep without interference from monitoring devices.

Furthermore, by tracing instant pressure readings of an air bladder and comparing the instant pressure readings to another value, an occupancy status of a bed may be determined and used in combination with one or more other data signals to accurately detect and prevent sleepwalking events. For example, as a trailing average approaches the instant pressure readings, a computer system may determine that a user is in the bed. Similarly, a smoothed pressure reading may be compared to an adjustable threshold and, if above the threshold, a computer system may determine that a user is in the bed. Coupled with sleep state information and historic sleepwalking data about the user, the computer system can accurately determine in real-time whether the user is likely to sleepwalk during a current sleep session. Based on this determination, the computer system can implement one or more interventions to prevent the user from sleepwalking and thus promote quality sleep and comfort during the current sleep session.

The details of one or more implementations are set forth in the accompanying drawings and the description below. Other features, aspects and potential advantages will be apparent from the accompanying description and figures.

### DESCRIPTION OF DRAWINGS

FIG. 1 shows an example air bed system.

FIG. 2 is a block diagram of an example of various components of an air bed system.

FIG. 3 shows an example environment including a bed in communication with devices located in and around a home.

FIGS. 4A and 4B are block diagrams of example data processing systems that can be associated with a bed.

FIGS. 5 and 6 are block diagrams of examples of motherboards that can be used in a data processing system associated with a bed.

FIG. 7 is a block diagram of an example of a daughterboard that can be used in a data processing system associated with a bed.

FIG. 8 is a block diagram of an example of a motherboard with no daughterboard that can be used in a data processing system associated with a bed.

FIG. 9 is a block diagram of an example of a sensory array that can be used in a data processing system associated with a bed.

FIG. 10 is a block diagram of an example of a control array that can be used in a data processing system associated with a bed

FIG. 11 is a block diagram of an example of a computing device that can be used in a data processing system associated with a bed.

FIGS. 12-16 are block diagrams of example cloud services that can be used in a data processing system associated with a bed.

FIG. 17 is a block diagram of an example of using a data processing system that can be associated with a bed to automate peripherals around the bed.

FIG. 18 is a schematic diagram that shows an example of a computing device and a mobile computing device.

FIG. 19 is a conceptual diagram for detecting and preventing sleepwalking events of a user in a bed system.

FIG. 20A is a flowchart of a process for detecting a sleepwalking event of a user.

FIG. 20B is a flowchart of another process for detecting a sleepwalking event of a user.

FIG. 21A is a flowchart of a process for preventing onset of a sleepwalking event of a user.

FIG. 21B is a flowchart of another process for preventing onset of a sleepwalking event of a user.

FIG. 22 is a system diagram of components of a computing system that can detect and prevent onset of a sleepwalking event of a user.

FIG. 23 is a swimlane diagram of an example process for training and using machine-learning classifiers to determine user sleep state, which can include sleep state.

FIG. 24 is a flowchart of an example process that may be used to train a sleep-stage classifier.

FIG. 25 is a flowchart of an example process for determining user presence in a bed.

FIG. 26 is a flowchart of another example process for determining user presence in a bed.

Like reference symbols in the various drawings indicate like elements.

### DETAILED DESCRIPTION

This disclosure generally describes technology that uses a bed to detect and prevent sleepwalking (e.g., somnambulism). To detect sleepwalking events, sensor data can be received by a computer system and from a sensor, such as a sensor integrated into the bed system and/or a sensor that is worn by the user (e.g., a wearable device, like a smart watch). The computer system can apply one or more machine learning models to the sensor data in order to classify sleep states of the user. The computer system can also use machine learning techniques to detect, from the sensor data, if and when the user exits the bed system. If the sleep state classification is a deep stage, such as N3, the user is detected as exiting the bed, and a total time from sleep onset is within 1 to 3 hours (e.g., 1 to 2 sleep cycles), the computer system can determine that a sleepwalking event has occurred. The computer system can store information about this detected sleepwalking event to be used during real-time prevention of sleepwalking onset.

To prevent the onset of somnambulism, the computer system can determine, during a given sleep session, whether a bed presence time of the user corresponds to the information that was stored about the detected sleepwalking event. The computer system can then determine a probability of sleepwalking onset within a threshold amount of time from a current time in the user's sleep session. Based on the probability, the computer system can perform an intervention to prevent the sleepwalking onset during the given sleep session. As described further below, the intervention can include adjusting a microclimate of the bed system by increasing a temperature of the microclimate such that the user's CBT decreases quickly and N3 sleep can be advanced. Another intervention can include providing sensory stimulation to delay N3 sleep. One or more other interventions may also be determined and performed.

### Example Airbed Hardware

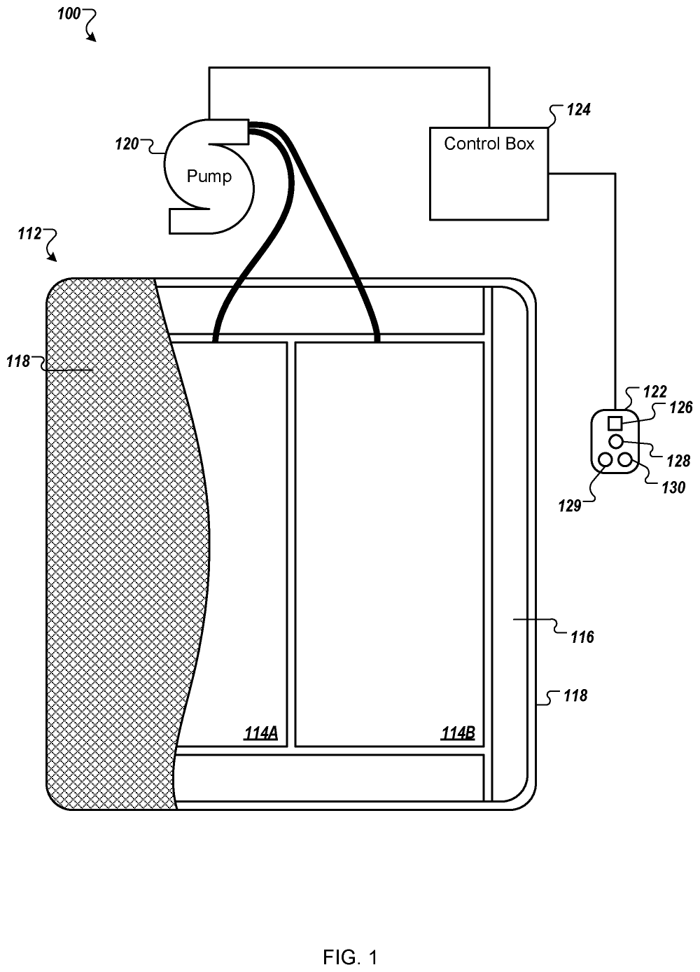

FIG. 1 shows an example air bed system 100 that includes a bed 112. The bed 112 can be a mattress that includes at least one air chamber 114 surrounded by a resilient border 116 and encapsulated by bed ticking 118. The resilient border 116 can comprise any suitable material, such as foam. In some embodiments, the resilient border 116 can combine with a top layer or layers of foam (not shown in FIG. 1) to form an upside down foam tub. In other embodiments, mattress structure can be varied as suitable for the application.

As illustrated in FIG. 1, the bed 112 can be a two chamber design having first and second fluid chambers, such as a first air chamber 114A and a second air chamber 114B. Sometimes, the bed 112 can include chambers for use with fluids other than air that are suitable for the application. For example, the fluids can include liquid. In some embodiments, such as single beds or kids' beds, the bed 112 can include a single air chamber 114A or 114B or multiple air chambers 114A and 114B. Although not depicted, sometimes, the bed 112 can include additional air chambers.

The first and second air chambers 114A and 114B can be in fluid communication with a pump 120. The pump 120 can be in electrical communication with a remote control 122 via control box 124. The control box 124 can include a wired or wireless communications interface for communicating with one or more devices, including the remote control 122. The control box 124 can be configured to operate the pump 120 to cause increases and decreases in the fluid pressure of the first and second air chambers 114A and 114B based upon commands input by a user using the remote control 122. In some implementations, the control box 124 is integrated into a housing of the pump 120. Moreover, sometimes, the pump 120 can be in wireless communication (e.g., via a home network, WIFI, BLUETOOTH, or other wireless network) with a mobile device via the control box 124. The mobile device can include but is not limited to the user's smartphone, cell phone, laptop, tablet, computer, wearable device, home automation device, or other computing device. A mobile application can be presented at the mobile device and provide functionality for the user to control the bed 112 and view information about the bed 112. The user can input commands in the mobile application presented at the mobile device. The inputted commands can be transmitted to the control box 124, which can operate the pump 120 based upon the commands.

The remote control 122 can include a display 126, an output selecting mechanism 128, a pressure increase button 129, and a pressure decrease button 130. The remote control 122 can include one or more additional output selecting mechanisms and/or buttons. The display 126 can present information to the user about settings of the bed 112. For example, the display 126 can present pressure settings of both the first and second air chambers 114A and 114B or one of the first and second air chambers 114A and 114B. Sometimes, the display 126 can be a touch screen, and can receive input from the user indicating one or more commands to control pressure in the first and second air chambers 114A and 114B and/or other settings of the bed 112.

The output selecting mechanism 128 can allow the user to switch air flow generated by the pump 120 between the first and second air chambers 114A and 114B, thus enabling control of multiple air chambers with a single remote control 122 and a single pump 120. For example, the output selecting mechanism 128 can by a physical control (e.g., switch or button) or an input control presented on the display 126. Alternatively, separate remote control units can be provided for each air chamber 114A and 114B and can each include the ability to control multiple air chambers. Pressure increase and decrease buttons 129 and 130 can allow the user to increase or decrease the pressure, respectively, in the air chamber selected with the output selecting mechanism 128. Adjusting the pressure within the selected air chamber can cause a corresponding adjustment to the firmness of the respective air chamber. In some embodiments, the remote control 122 can be omitted or modified as appropriate for an application. For example, as mentioned above, the bed 112 can be controlled by a mobile device in wired or wireless communication with the bed 112.

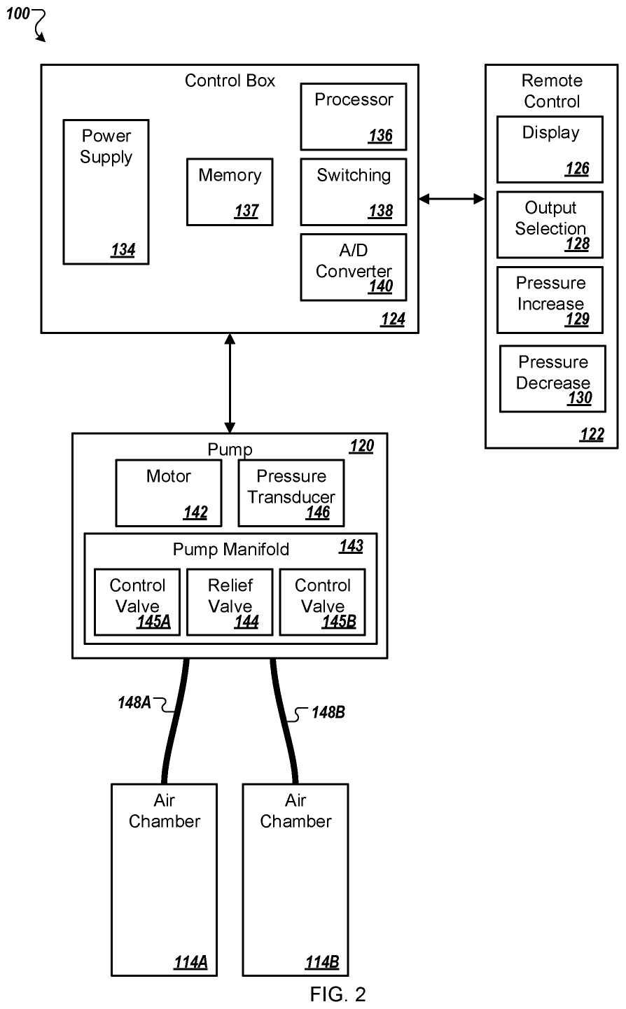

FIG. 2 is a block diagram of an example of various components of an air bed system. For example, these components can be used in the example air bed system 100. As shown in FIG. 2, the control box 124 can include a power supply 134, a processor 136, a memory 137, a switching mechanism 138, and an analog to digital (A/D) converter 140. The switching mechanism 138 can be, for example, a relay or a solid state switch. In some implementations, the switching mechanism 138 can be located in the pump 120 rather than the control box 124.

The pump 120 and the remote control 122 can be in two-way communication with the control box 124. The pump 120 includes a motor 142, a pump manifold 143, a relief valve 144, a first control valve 145A, a second control valve 145B, and a pressure transducer 146. The pump 120 is fluidly connected with the first air chamber 114A and the second air chamber 114B via a first tube 148A and a second tube 148B, respectively. The first and second control valves 145A and 145B can be controlled by switching mechanism 138, and are operable to regulate the flow of fluid between the pump 120 and first and second air chambers 114A and 114B, respectively.

In some implementations, the pump 120 and the control box 124 can be provided and packaged as a single unit. In some implementations, the pump 120 and the control box 124 can be provided as physically separate units. In yet some implementations, the control box 124, the pump 120, or both can be integrated within or otherwise contained within a bed frame, foundation, or bed support structure that supports the bed 112. Sometimes, the control box 124, the pump 120, or both can be located outside of a bed frame, foundation, or bed support structure (as shown in the example in FIG. 1).

The example air bed system 100 depicted in FIG. 2 includes the two air chambers 114A and 114B and the single pump 120 of the bed 112 depicted in FIG. 1. However, other implementations can include an air bed system having two or more air chambers and one or more pumps incorporated into the air bed system to control the air chambers. For example, a separate pump can be associated with each air chamber of the air bed system. As another example, a pump can be associated with multiple chambers of the air bed system. A first pump can, for example, be associated with air chambers that extend longitudinally from a left side to a midpoint of the air bed system 100 and a second pump can be associated with air chambers that extend longitudinally from a right side to the midpoint of the air bed system 100. Separate pumps can allow each air chamber to be inflated or deflated independently and/or simultaneously. Furthermore, additional pressure transducers can be incorporated into the air bed system 100 such that, for example, a separate pressure transducer can be associated with each air chamber.

As an illustrative example, in use, the processor 136 can send a decrease pressure command to one of air chambers 114A or 114B, and the switching mechanism 138 can convert the low voltage command signals sent by the processor 136 to higher operating voltages sufficient to operate the relief valve 144 of the pump 120 and open the respective control valve 145A or 145B. Opening the relief valve 144 can allow air to escape from the air chamber 114A or 114B through the respective air tube 148A or 148B. During deflation, the pressure transducer 146 can send pressure readings to the processor 136 via the A/D converter 140. The A/D converter 140 can receive analog information from pressure transducer 146 and can convert the analog information to digital information useable by the processor 136. The processor 136 can send the digital signal to the remote control 122 to update the display 126 in order to convey the pressure information to the user. The processor 136 can also send the digital signal to one or more other devices in wired or wireless communication with the air bed system, including but not limited to mobile devices such as smartphones, cellphones, tablets, computers, wearable devices, and home automation devices. As a result, the user can view pressure information associated with the air bed system at their mobile device instead of at, or in addition to, the remote control 122.

As another example, the processor 136 can send an increase pressure command. The pump motor 142 can be energized in response to the increase pressure command and send air to the designated one of the air chambers 114A or 114B through the air tube 148A or 148B via electronically operating the corresponding valve 145A or 145B. While air is being delivered to the designated air chamber 114A or 114B in order to increase the firmness of the chamber, the pressure transducer 146 can sense pressure within the pump manifold 143. Again, the pressure transducer 146 can send pressure readings to the processor 136 via the A/D converter 140. The processor 136 can use the information received from the A/D converter 140 to determine the difference between the actual pressure in air chamber 114A or 114B and the desired pressure. The processor 136 can send the digital signal to the remote control 122 to update display 126 in order to convey the pressure information to the user.

Generally speaking, during an inflation or deflation process, the pressure sensed within the pump manifold 143 can provide an approximation of the pressure within the respective air chamber that is in fluid communication with the pump manifold 143. An example method of obtaining a pump manifold pressure reading that is substantially equivalent to the actual pressure within an air chamber includes turning off the pump 120, allowing the pressure within the air chamber 114A or 114B and the pump manifold 143 to equalize, and then sensing the pressure within the pump manifold 143 with the pressure transducer 146. Thus, providing a sufficient amount of time to allow the pressures within the pump manifold 143 and chamber 114A or 114B to equalize can result in pressure readings that are accurate approximations of actual pressure within air chamber 114A or 114B. In some implementations, the pressure of the air chambers 114A and/or 114B can be continuously monitored using multiple pressure sensors (not shown). The pressure sensors can be positioned within the air chambers 114A and/or 114B. The pressure sensors can also be fluidly connected to the air chambers 114A and 114B, such as along the air tubes 148A and 148B.

In some implementations, information collected by the pressure transducer 146 can be analyzed to determine various states of a user laying on the bed 112. For example, the processor 136 can use information collected by the pressure transducer 146 to determine a heartrate or a respiration rate for the user laying on the bed 112. As an illustrative example, the user can be laying on a side of the bed 112 that includes the chamber 114A. The pressure transducer 146 can monitor fluctuations in pressure of the chamber 114A, and this information can be used to determine the user's heartrate and/or respiration rate. As another example, additional processing can be performed using the collected data to determine a sleep state of the user (e.g., awake, light sleep, deep sleep). For example, the processor 136 can determine when the user falls asleep and, while asleep, the various sleep states (e.g., sleep states) of the user. Based on the determined heartrate, respiration rate, and/or sleep states of the user, the processor 136 can determine information about the user's sleep quality. The processor 136 can, for example, determine how well the user slept during a particular sleep cycle. The processor 136 can also determine user sleep cycle trends. Accordingly, the processor 136 can generate recommendations to improve the user's sleep quality and overall sleep cycle. Information that is determined about the user's sleep cycle (e.g., heartrate, respiration rate, sleep states, sleep quality, recommendations to improve sleep quality, etc.) can be transmitted to the user's mobile device and presented in a mobile application, as described above.

Additional information associated with the user of the air bed system 100 that can be determined using information collected by the pressure transducer 146 includes motion of the user, presence of the user on a surface of the bed 112, weight of the user, heart arrhythmia of the user, snoring of the user or another user on the air bed system, and apnea of the user. One or more other health conditions of the user can also be determined based on the information collected by the pressure transducer 146. Taking user presence detection for example, the pressure transducer 146 can be used to detect the user's presence on the bed 112, e.g., via a gross pressure change determination and/or via one or more of a respiration rate signal, heartrate signal, and/or other biometric signals. Detection of the user's presence on the bed 112 can be beneficial to determine, by the processor 136, one or more adjustments to make to settings of the bed 112 (e.g., adjusting a firmness of the bed 112 when the user is present to a user-preferred firmness setting) and/or peripheral devices (e.g., turning off lights when the user is present, activating a heating or cooling system, etc.).

For example, a simple pressure detection process can identify an increase in pressure as an indication that the user is present on the bed 112. As another example, the processor 136 can determine that the user is present on the bed 112 if the detected pressure increases above a specified threshold (so as to indicate that a person or other object above a certain weight is positioned on the bed 112). As yet another example, the processor 136 can identify an increase in pressure in combination with detected slight, rhythmic fluctuations in pressure as corresponding to the user being present on the bed 112. The presence of rhythmic fluctuations can be identified as being caused by respiration or heart rhythm (or both) of the user. The detection of respiration or a heartbeat can distinguish between the user being present on the bed and another object (e.g., a suitcase, a pet, a pillow, etc.) being placed upon the bed.

In some implementations, fluctuations in pressure can be measured at the pump 120. For example, one or more pressure sensors can be located within one or more internal cavities of the pump 120 to detect fluctuations in pressure within the pump 120. The fluctuations in pressure detected at the pump 120 can indicate fluctuations in pressure in one or both of the chambers 114A and 114B. One or more sensors located at the pump 120 can be in fluid communication with one or both of the chambers 114A and 114B, and the sensors can be operative to determine pressure within the chambers 114A and 114B. The control box 124 can be configured to determine at least one vital sign (e.g., heartrate, respiratory rate) based on the pressure within the chamber 114A or the chamber 114B.

In some implementations, the control box 124 can analyze a pressure signal detected by one or more pressure sensors to determine a heartrate, respiration rate, and/or other vital signs of the user lying or sitting on the chamber 114A and/or 114B. More specifically, when a user lies on the bed 112 and is positioned over the chamber 114A, each of the user's heart beats, breaths, and other movements (e.g., hand, arm, leg, foot, or other gross body movements) can create a force on the bed 112 that is transmitted to the chamber 114A. As a result of the force input applied to the chamber 114A from the user's movement, a wave can propagate through the chamber 114A and into the pump 120. A pressure sensor located at the pump 120 can detect the wave, and thus the pressure signal outputted by the sensor can indicate a heartrate, respiratory rate, or other information regarding the user.

With regard to sleep state, the air bed system 100 can determine the user's sleep state by using various biometric signals such as heartrate, respiration, and/or movement of the user. While the user is sleeping, the processor 136 can receive one or more of the user's biometric signals (e.g., heartrate, respiration, motion, etc.) and can determine the user's present sleep state based on the received biometric signals. In some implementations, signals indicating fluctuations in pressure in one or both of the chambers 114A and 114B can be amplified and/or filtered to allow for more precise detection of heartrate and respiratory rate.

Sometimes, the processor 136 can also receive additional biometric signals of the user from one or more other sensors or sensor arrays that are positioned on or otherwise integrated into the air bed system 100. For example, one or more sensors can be attached or removably attached to a top surface of the air bed system 100 and configured to detect signals such as heartrate, respiration rate, and/or motion of the user. The processor 136 can then combine biometric signals received from pressure sensors located at the pump 120, the pressure transducer 146, and/or the sensors positioned throughout the air bed system 100 to generate accurate and more precise heartrate, respiratory rate, and other information about the user and the user's sleep quality.

Sometimes, the control box 124 can perform a pattern recognition algorithm or other calculation based on the amplified and filtered pressure signal(s) to determine the user's heartrate and/or respiratory rate. For example, the algorithm or calculation can be based on assumptions that a heartrate portion of the signal has a frequency in a range of 0.5-4.0 Hz and that a respiration rate portion of the signal has a frequency in a range of less than 1 Hz. Sometimes, the control box 124 can use one or more machine learning models to determine the user's heartrate, respiratory rate, or other health information. The models can be trained using training data that includes training pressure signals and expected heartrates and/or respiratory rates. Sometimes, the control box 124 can determine the user's heartrate, respiratory rate, or other health information by using a lookup table that corresponds to sensed pressure signals.

The control box 124 can also be configured to determine other characteristics of the user based on the received pressure signal, such as blood pressure, tossing and turning movements, rolling movements, limb movements, weight, presence or lack of presence of the user, and/or the identity of the user.

For example, the pressure transducer 146 can be used to monitor the air pressure in the chambers 114A and 114B of the bed 112. If the user on the bed 112 is not moving, the air pressure changes in the air chamber 114A or 114B can be relatively minimal, and can be attributable to respiration and/or heartbeat. When the user on the bed 112 is moving, however, the air pressure in the mattress can fluctuate by a much larger amount. Thus, the pressure signals generated by the pressure transducer 146 and received by the processor 136 can be filtered and indicated as corresponding to motion, heartbeat, or respiration. The processor 136 can also attribute such fluctuations in air pressure to sleep quality of the user. Such attributions can be determined based on applying one or more machine learning models and/or algorithms to the pressure signals generated by the pressure transducer 146. For example, if the user shifts and turns a lot during a sleep cycle (for example, in comparison to historic trends of the user's sleep cycles), the processor 136 can determine that the user experienced poor sleep during that particular sleep cycle.

In some implementations, rather than performing the data analysis in the control box 124 with the processor 136, a digital signal processor (DSP) can be provided to analyze the data collected by the pressure transducer 146. Alternatively, the data collected by the pressure transducer 146 can be sent to a cloud-based computing system for remote analysis.

In some implementations, the example air bed system 100 further includes a temperature controller configured to increase, decrease, or maintain a temperature of the bed 112, for example for the comfort of the user. For example, a pad (e.g., mat, layer, etc.) can be placed on top of or be part of the bed 112, or can be placed on top of or be part of one or both of the chambers 114A and 114B. Air can be pushed through the pad and vented to cool off the user on the bed 112. Additionally or alternatively, the pad can include a heating element that can be used to keep the user warm. In some implementations, the temperature controller can receive temperature readings from the pad. The temperature controller can determine whether the temperature readings are less than or greater than some threshold range and/or value. Based on this determination, the temperature controller can actuate components to push air through the pad to cool off the user or active the heating element. In some implementations, separate pads are used for different sides of the bed 112 (e.g., corresponding to the locations of the chambers 114A and 114B) to provide for differing temperature control for the different sides of the bed 112. Each pad can therefore be selectively controlled by the temperature controller to provide cooling or heating that is preferred by each of the users on the different sides of the bed 112. For example, a first user on a left side of the bed 112 can prefer to have their side of the bed 112 cooled during the night while a second user on a right side of the bed 112 can prefer to have their side of the bed 112 warmed during the night.

In some implementations, the user of the air bed system 100 can use an input device, such as the remote control 122 or a mobile device as described above, to input a desired temperature for a surface of the bed 112 (or for a portion of the surface of the bed 112, for example at a foot region, a lumbar or waist region, a shoulder region, and/or a head region of the bed 112). The desired temperature can be encapsulated in a command data structure that includes the desired temperature and also identifies the temperature controller as the desired component to be controlled. The command data structure can then be transmitted via Bluetooth or another suitable communication protocol (e.g., WIFI, a local network, etc.) to the processor 136. In various examples, the command data structure is encrypted before being transmitted. The temperature controller can then configure its elements to increase or decrease the temperature of the pad depending on the temperature input provided at the remote control 122 by the user.

In some implementations, data can be transmitted from a component back to the processor 136 or to one or more display devices, such as the display 126 of the remote controller 122. For example, the current temperature as determined by a sensor element of temperature controller, the pressure of the bed, the current position of the foundation or other information can be transmitted to control box 124. The control box 124 can then transmit the received information to the remote control 122, where the information can be displayed to the user (e.g., on the display 126). As described above, the control box 124 can also transmit the received information to a mobile device (e.g., smartphone, cellphone, laptop, tablet, computer, wearable device, or home automation device) to be displayed in a mobile application or other graphical user interface (GUI) to the user.

In some implementations, the example air bed system 100 further includes an adjustable foundation and an articulation controller configured to adjust the position of a bed (e.g., the bed 112) by adjusting the adjustable foundation that supports the bed. For example, the articulation controller can adjust the bed 112 from a flat position to a position in which a head portion of a mattress of the bed is inclined upward (e.g., to facilitate a user sitting up in bed and/or watching television). The bed 112 can also include multiple separately articulable sections. As an illustrative example, the bed 112 can include one or more of a head portion, a lumbar/waist portion, a leg portion, and/or a foot portion, all of which can be separately articulable. As another example, portions of the bed 112 corresponding to the locations of the chambers 114A and 114B can be articulated independently from each other, to allow one user positioned on the bed 112 surface to rest in a first position (e.g., a flat position or other desired position) while a second user rests in a second position (e.g., a reclining position with the head raised at an angle from the waist or another desired position). Separate positions can also be set for two different beds (e.g., two twin beds placed next to each other). The foundation of the bed 112 can include more than one zone that can be independently adjusted.

Sometimes, the bed 112 can be adjusted to one or more user-defined positions based on user input and/or user preferences. For example, the bed 112 can automatically adjust, by the articulation controller, to one or more user-defined settings. As another example, the user can control the articulation controller to adjust the bed 112 to one or more user-defined positions. Sometimes, the bed 112 can be adjusted to one or more positions that may provide the user with improved or otherwise improve sleep and sleep quality. For example, a head portion on one side of the bed 112 can be automatically articulated, by the articulation controller, when one or more sensors of the air bed system 100 detect that a user sleeping on that side of the bed 112 is snoring. As a result, the user's snoring can be mitigated so that the snoring does not wake up another user sleeping in the bed 112.

In some implementations, the bed 112 can be adjusted using one or more devices in communication with the articulation controller or instead of the articulation controller. For example, the user can change positions of one or more portions of the bed 112 using the remote control 122 described above. The user can also adjust the bed 112 using a mobile application or other graphical user interface presented at a mobile computing device of the user.

The articulation controller can also be configured to provide different levels of massage to one or more portions of the bed 112 for one or more users on the bed 112. The user(s) can also adjust one or more massage settings for different portions of the bed 112 using the remote control 122 and/or a mobile device in communication with the air bed system 100, as described above.

### Example of a Bed in a Bedroom Environment

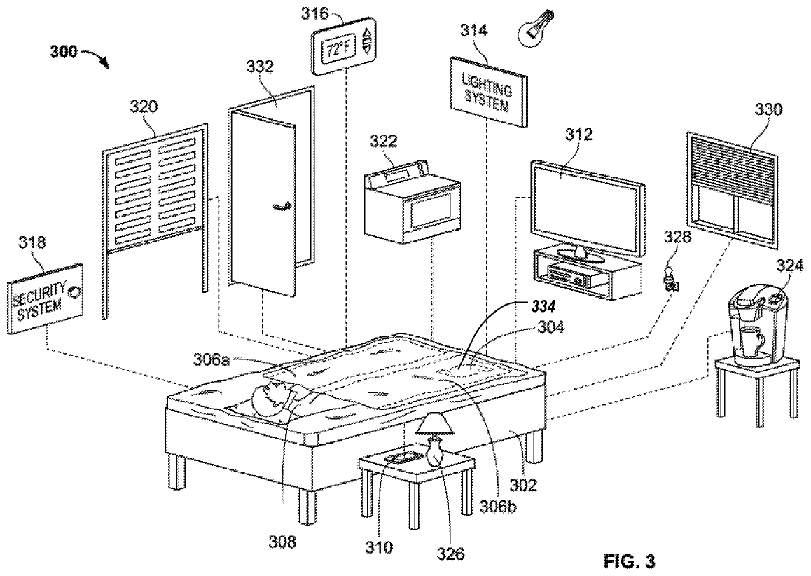

FIG. 3 shows an example environment 300 including a bed 302 in communication with devices located in and around a home. In the example shown, the bed 302 includes pump 304 for controlling air pressure within two air chambers 306a and 306b (as described above with respect to the air chambers 114A and 114B). The pump 304 additionally includes circuitry 334 for controlling inflation and deflation functionality performed by the pump 304. The circuitry 334 is further programmed to detect fluctuations in air pressure of the air chambers 306a-b and uses the detected fluctuations in air pressure to identify bed presence of a user 308, sleep state of the user 308, movement of the user 308, and biometric signals of the user 308, such as heartrate and respiration rate. The detected fluctuations in air pressure can also be used to detect when the user 308 is snoring and whether the user 308 has sleep apnea or other health conditions. Moreover, the detected fluctuations in air pressure can be used to determine an overall sleep quality of the user 308.

In the example shown, the pump 304 is located within a support structure of the bed 302 and the control circuitry 334 for controlling the pump 304 is integrated with the pump 304. In some implementations, the control circuitry 334 is physically separate from the pump 304 and is in wireless or wired communication with the pump 304. In some implementations, the pump 304 and/or control circuitry 334 are located outside of the bed 302. In some implementations, various control functions can be performed by systems located in different physical locations. For example, circuitry for controlling actions of the pump 304 can be located within a pump casing of the pump 304 while control circuitry 334 for performing other functions associated with the bed 302 can be located in another portion of the bed 302, or external to the bed 302. As another example, the control circuitry 334 located within the pump 304 can communicate with control circuitry 334 at a remote location through a LAN or WAN (e.g., the internet). As yet another example, the control circuitry 334 can be included in the control box 124 of FIGS. 1 and 2.

In some implementations, one or more devices other than, or in addition to, the pump 304 and control circuitry 334 can be utilized to identify user bed presence, sleep state, movement, biometric signals, and other information (e.g., sleep quality and/or health related) about the user 308. For example, the bed 302 can include a second pump in addition to the pump 304, with each of the two pumps connected to a respective one of the air chambers 306a-b. For example, the pump 304 can be in fluid communication with the air chamber 306b to control inflation and deflation of the air chamber 306b as well as detect user signals for a user located over the air chamber 306b, such as bed presence, sleep state, movement, and biometric signals. The second pump can then be in fluid communication with the air chamber 306a and used to control inflation and deflation of the air chamber 306a as well as detect user signals for a user located over the air chamber 306a.

As another example, the bed 302 can include one or more pressure sensitive pads or surface portions that are operable to detect movement, including user presence, user motion, respiration, and heartrate. A first pressure sensitive pad can be incorporated into a surface of the bed 302 over a left portion of the bed 302, where a first user would normally be located during sleep, and a second pressure sensitive pad can be incorporated into the surface of the bed 302 over a right portion of the bed 302, where a second user would normally be located during sleep. The movement detected by the one or more pressure sensitive pads or surface portions can be used by control circuitry 334 to identify user sleep state, bed presence, or biometric signals for each of the users. The pressure sensitive pads can also be removable rather than incorporated into the surface of the bed 302.

The bed 302 can also include one or more temperature sensors and/or array of sensors that are operable to detect temperatures in microclimates of the bed 302. Detected temperatures in different microclimates of the bed 302 can be used by the control circuitry 334 to determine one or more modifications to the user 308's sleep environment. For example, a temperature sensor located near a core region of the bed 302 where the user 308 rests can detect high temperature values. Such high temperature values can indicate that the user 308 is warm. To lower the user's body temperature in this microclimate, the control circuitry 334 can determine that a cooling element of the bed 302 can be activated. As another example, the control circuitry 334 can determine that a cooling unit in the home can be automatically activated to cool an ambient temperature in the environment 300.

The control circuitry 334 can also process a combination of signals sensed by different sensors that are integrated into, positioned on, or otherwise in communication with the bed 112. For example, pressure and temperature signals can be processed by the control circuitry 334 to more accurately determine one or more health conditions of the user 308 and/or sleep quality of the user 308. Acoustic signals detected by one or more microphones or other audio sensors can also be used in combination with pressure or motion sensors in order to determine when the user 308 snores, whether the user 308 has sleep apnea, and/or overall sleep quality of the user 308. Combinations of one or more other sensed signals are also possible for the control circuitry 334 to more accurately determine one or more health and/or sleep conditions of the user 308.

Accordingly, information detected by one or more sensors or other components of the bed 112 (e.g., motion information) can be processed by the control circuitry 334 and provided to one or more user devices, such as a user device 310 for presentation to the user 308 or to other users. The information can be presented in a mobile application or other graphical user interface at the user device 310. The user 308 can view different information that is processed and/or determined by the control circuitry 334 and based the signals that are detected by components of the bed 302. For example, the user 308 can view their overall sleep quality for a particular sleep cycle (e.g., the previous night), historic trends of their sleep quality, and health information. The user 308 can also adjust one or more settings of the bed 302 (e.g., increase or decrease pressure in one or more regions of the bed 302, incline or decline different regions of the bed 302, turn on or off massage features of the bed 302, etc.) using the mobile application that is presented at the user device 310.

In the example depicted in FIG. 3, the user device 310 is a mobile phone; however, the user device 310 can also be any one of a tablet, personal computer, laptop, a smartphone, a smart television (e.g., a television 312), a home automation device, or other user device capable of wired or wireless communication with the control circuitry 334, one or more other components of the bed 302, and/or one or more devices in the environment 300. The user device 310 can be in communication with the control circuitry 334 of the bed 302 through a network or through direct point-to-point communication. For example, the control circuitry 334 can be connected to a LAN (e.g., through a WIFI router) and communicate with the user device 310 through the LAN. As another example, the control circuitry 334 and the user device 310 can both connect to the Internet and communicate through the Internet. For example, the control circuitry 334 can connect to the Internet through a WIFI router and the user device 310 can connect to the Internet through communication with a cellular communication system. As another example, the control circuitry 334 can communicate directly with the user device 310 through a wireless communication protocol, such as Bluetooth. As yet another example, the control circuitry 334 can communicate with the user device 310 through a wireless communication protocol, such as ZigBee, Z-Wave, infrared, or another wireless communication protocol suitable for the application. As another example, the control circuitry 334 can communicate with the user device 310 through a wired connection such as, for example, a USB connector, serial/RS232, or another wired connection suitable for the application.

As mentioned above, the user device 310 can display a variety of information and statistics related to sleep, or user 308's interaction with the bed 302. For example, a user interface displayed by the user device 310 can present information including amount of sleep for the user 308 over a period of time (e.g., a single evening, a week, a month, etc.), amount of deep sleep, ratio of deep sleep to restless sleep, time lapse between the user 308 getting into bed and the user 308 falling asleep, total amount of time spent in the bed 302 for a given period of time, heartrate for the user 308 over a period of time, respiration rate for the user 308 over a period of time, or other information related to user interaction with the bed 302 by the user 308 or one or more other users of the bed 302. In some implementations, information for multiple users can be presented on the user device 310, for example information for a first user positioned over the air chamber 306a can be presented along with information for a second user positioned over the air chamber 306b. In some implementations, the information presented on the user device 310 can vary according to the age of the user 308. For example, the information presented on the user device 310 can evolve with the age of the user 308 such that different information is presented on the user device 310 as the user 308 ages as a child or an adult.

The user device 310 can also be used as an interface for the control circuitry 334 of the bed 302 to allow the user 308 to enter information and/or adjust one or more settings of the bed 302. The information entered by the user 308 can be used by the control circuitry 334 to provide better information to the user 308 or to various control signals for controlling functions of the bed 302 or other devices. For example, the user 308 can enter information such as weight, height, and age of the user 308. The control circuitry 334 can use this information to provide the user 308 with a comparison of the user 308's tracked sleep information to sleep information of other people having similar weights, heights, and/or ages as the user 308. The control circuitry 308 can also use this information to more accurately determine overall sleep quality and/or health of the user 308 based on information that is detected by one or more components (e.g., sensors) of the bed 302.

As another example, and as mentioned above, the user 308 can use the user device 310 as an interface for controlling air pressure of the air chambers 306a and 306b, for controlling various recline or incline positions of the bed 302, for controlling temperature of one or more surface temperature control devices of the bed 302, or for allowing the control circuitry 334 to generate control signals for other devices (as described in greater detail below).

In some implementations, the control circuitry 334 of the bed 302 can communicate with other devices or systems in addition to or instead of the user device 310. For example, the control circuitry 334 can communicate with the television 312, a lighting system 314, a thermostat 316, a security system 318, home automation devices, and/or other household devices, including but not limited to an oven 322, a coffee maker 324, a lamp 326, and/or a nightlight 328. Other examples of devices and/or systems that the control circuitry 334 can communicate with include a system for controlling window blinds 330, one or more devices for detecting or controlling the states of one or more doors 332 (such as detecting if a door is open, detecting if a door is locked, or automatically locking a door), and a system for controlling a garage door 320 (e.g., control circuitry 334 integrated with a garage door opener for identifying an open or closed state of the garage door 320 and for causing the garage door opener to open or close the garage door 320). Communications between the control circuitry 334 of the bed 302 and other devices can occur through a network (e.g., a LAN or the Internet) or as point-to-point communication (e.g., using Bluetooth, radio communication, or a wired connection). In some implementations, control circuitry 334 of different beds 302 can communicate with different sets of devices. For example, a kid's bed may not communicate with and/or control the same devices as an adult bed. In some embodiments, the bed 302 can evolve with the age of the user such that the control circuitry 334 of the bed 302 communicates with different devices as a function of age of the user of that bed 302.

The control circuitry 334 can receive information and inputs from other devices/systems and use the received information and inputs to control actions of the bed 302 and/or other devices. For example, the control circuitry 334 can receive information from the thermostat 316 indicating a current environmental temperature for a house or room in which the bed 302 is located. The control circuitry 334 can use the received information (along with other information, such as signals detected from one or more sensors of the bed 302) to determine if a temperature of all or a portion of the surface of the bed 302 should be raised or lowered. The control circuitry 334 can then cause a heating or cooling mechanism of the bed 302 to raise or lower the temperature of the surface of the bed 302. The control circuitry 334 can also cause a heating or cooling unit of the house or room in which the bed 302 is located to raise or lower the ambient temperature surrounding the bed 302. Thus, by adjusting the temperature of the bed 302 and/or the room in which the bed 302 is located, the user 308 can experience more improved sleep quality and comfort.

As an example, the user 308 can indicate a desired sleeping temperature of 74 degrees while a second user of the bed 302 indicates a desired sleeping temperature of 72 degrees. The thermostat 316 can transmit signals indicating room temperature at predetermined times to the control circuitry 334. The thermostat 316 can also send a continuous stream of detected temperature values of the room to the control circuitry 334. The transmitted signal(s) can indicate to the control circuitry 334 that the current temperature of the bedroom is 72 degrees. The control circuitry 334 can identify that the user 308 has indicated a desired sleeping temperature of 74 degrees, and can accordingly send control signals to a heating pad located on the user 308's side of the bed to raise the temperature of the portion of the surface of the bed 302 where the user 308 is located until the user 308's desired temperature is achieved. Moreover, the control circuitry 334 can sent control signals to the thermostat 316 and/or a heating unit in the house to raise the temperature in the room in which the bed 302 is located.

The control circuitry 334 can generate control signals to control other devices and propagate the control signals to the other devices. In some implementations, the control signals are generated based on information collected by the control circuitry 334, including information related to user interaction with the bed 302 by the user 308 and/or one or more other users. Information collected from one or more other devices other than the bed 302 can also be used when generating the control signals. For example, information relating to environmental occurrences (e.g., environmental temperature, environmental noise level, and environmental light level), time of day, time of year, day of the week, or other information can be used when generating control signals for various devices in communication with the control circuitry 334 of the bed 302.

For example, information on the time of day can be combined with information relating to movement and bed presence of the user 308 to generate control signals for the lighting system 314. The control circuitry 334 can, based on detected pressure signals of the user 308 on the bed 302, determine when the user 308 is presently in the bed 302 and when the user 308 falls asleep. Once the control circuitry 334 determines that the user has fallen asleep, the control circuitry 334 can transmit control signals to the lighting system 314 to turn off lights in the room in which the bed 302 is located, to lower the window blinds 330 in the room, and/or to activate the nightlight 328. Moreover, the control circuitry 334 can receive input from the user 308 (e.g., via the user device 310) that indicates a time at which the user 308 would like to wake up. When that time approaches, the control circuitry 334 can transmit control signals to one or more devices in the environment 300 to control devices that may cause the user 308 to wake up. For example, the control signals can be sent to a home automation device that controls multiple devices in the home. The home automation device can be instructed, by the control circuitry 334, to raise the window blinds 330, turn off the nightlight 328, turn on lighting beneath the bed 302, start the coffee machine 324, change a temperature in the house via the thermostat 316, or perform some other home automation. The home automation device can also be instructed to activate an alarm that can cause the user 308 to wake up. Sometimes, the user 308 can input information at the user device 310 that indicates what actions can be taken by the home automation device or other devices in the environment 300.

In some implementations, rather than or in addition to providing control signals for one or more other devices, the control circuitry 334 can provide collected information (e.g., information related to user movement, bed presence, sleep state, or biometric signals for the user 308) to one or more other devices to allow the one or more other devices to utilize the collected information when generating control signals. For example, the control circuitry 334 of the bed 302 can provide information relating to user interactions with the bed 302 by the user 308 to a central controller (not shown) that can use the provided information to generate control signals for various devices, including the bed 302.

The central controller can, for example, be a hub device that provides a variety of information about the user 308 and control information associated with the bed 302 and one or more other devices in the house. The central controller can include one or more sensors that detect signals that can be used by the control circuitry 334 and/or the central controller to determine information about the user 308 (e.g., biometric or other health data, sleep quality, etc.). The sensors can detect signals including but not limited to ambient light, temperature, humidity, volatile organic compound(s), pulse, motion, and audio. These signals can be combined with signals that are detected by sensors of the bed 302 to determine more accurate information about the user 308's health and sleep quality. The central controller can provide controls (e.g., user-defined, presets, automated, user initiated, etc.) for the bed 302, determining and viewing sleep quality and health information, a smart alarm clock, a speaker or other home automation device, a smart picture frame, a nightlight, and one or more mobile applications that the user 308 can install and use at the central controller. The central controller can include a display screen that can output information and also receive input from the user 308. The display can output information such as the user 308's health, sleep quality, weather information, security integration features, lighting integration features, heating and cooling integration features, and other controls to automate devices in the house. The central controller can therefore operate to provide the user 308 with functionality and control of multiple different types of devices in the house as well as the user 308's bed 302.

Still referring to FIG. 3, the control circuitry 334 of the bed 302 can generate control signals for controlling actions of other devices, and transmit the control signals to the other devices in response to information collected by the control circuitry 334, including bed presence of the user 308, sleep state of the user 308, and other factors. For example, the control circuitry 334 integrated with the pump 304 can detect a feature of a mattress of the bed 302, such as an increase in pressure in the air chamber 306b, and use this detected increase in air pressure to determine that the user 308 is present on the bed 302. In some implementations, the control circuitry 334 can identify a heartrate or respiratory rate for the user 308 to identify that the increase in pressure is due to a person sitting, laying, or otherwise resting on the bed 302, rather than an inanimate object (such as a suitcase) having been placed on the bed 302. In some implementations, the information indicating user bed presence can be combined with other information to identify a current or future likely state for the user 308. For example, a detected user bed presence at 11:00 am can indicate that the user is sitting on the bed (e.g., to tie her shoes, or to read a book) and does not intend to go to sleep, while a detected user bed presence at 10:00 pm can indicate that the user 308 is in bed for the evening and is intending to fall asleep soon. As another example, if the control circuitry 334 detects that the user 308 has left the bed 302 at 6:30 am (e.g., indicating that the user 308 has woken up for the day), and then later detects presence of the user 308 at 7:30 am on the bed 302, the control circuitry 334 can use this information that the newly detected presence is likely temporary (e.g., while the user 308 ties her shoes before heading to work) rather than an indication that the user 308 is intending to stay on the bed 302 for an extended period of time.

If the control circuitry 334 determines that the user 308 is likely to remain on the bed 302 for an extended period of time, the control circuitry 334 can determine one or more home automation controls that can aid the user 308 in falling asleep and experiencing improved sleep quality throughout the user 308's sleep cycle. For example, the control circuitry 334 can communicate with security system 318 to ensure that doors are locked. The control circuitry 334 can communicate with the oven 322 to ensure that the oven 322 is turned off. The control circuitry 334 can also communicate with the lighting system 314 to dim or otherwise turn off lights in the room in which the bed 302 is located and/or throughout the house, and the control circuitry 334 can communicate with the thermostat 316 to ensure that the house is at a desired temperature of the user 308. The control circuitry 334 can also determine one or more adjustments that can be made to the bed 302 to facilitate the user 308 falling asleep and staying asleep (e.g., changing a position of one or more regions of the bed 302, foot warming, massage features, pressure/firmness in one or more regions of the bed 302, etc.).

In some implementations, the control circuitry 334 is able to use collected information (including information related to user interaction with the bed 302 by the user 308, as well as environmental information, time information, and input received from the user 308) to identify use patterns for the user 308. For example, the control circuitry 334 can use information indicating bed presence and sleep states for the user 308 collected over a period of time to identify a sleep pattern for the user. The control circuitry 334 can identify that the user 308 generally goes to bed between 9:30 pm and 10:00 pm, generally falls asleep between 10:00 μm and 11:00 μm, and generally wakes up between 6:30 am and 6:45 am, based on information indicating user presence and biometrics for the user 308 collected over a week or a different time period. The control circuitry 334 can use identified patterns of the user 308 to better process and identify user interactions with the bed 302.

For example, given the above example user bed presence, sleep, and wake patterns for the user 308, if the user 308 is detected as being on the bed 302 at 3:00 pm, the control circuitry 334 can determine that the user 308's presence on the bed 302 is only temporary, and use this determination to generate different control signals than would be generated if the control circuitry 334 determined that the user 308 was in bed for the evening (e.g., at 3:00 pm, a head region of the bed 302 can be raised to facilitate reading or watching TV while in the bed 302, whereas in the evening, the bed 302 can be adjusted to a flat position to facilitate falling asleep). As another example, if the control circuitry 334 detects that the user 308 has gotten out of bed at 3:00 am, the control circuitry 334 can use identified patterns for the user 308 to determine that the user has only gotten up temporarily (e.g., to use the bathroom, or get a glass of water) and is not up for the day. For example, the control circuitry 334 can turn on underbed lighting to assist the user 308 in carefully moving around the bed 302 and the room. By contrast, if the control circuitry 334 identifies that the user 308 has gotten out of the bed 302 at 6:40 am, the control circuitry 334 can determine that the user 308 is up for the day and generate a different set of control signals than those that would be generated if it were determined that the user 308 were only getting out of bed temporarily (as would be the case when the user 308 gets out of the bed 302 at 3:00 am) (e.g., the control circuitry 334 can turn on light 326 near the bed 302 and/or raise the window blinds 330 when it is determined that the user 308 is up for the day). For other users, getting out of the bed 302 at 3:00 am can be a normal wake-up time, which the control circuitry 334 can learn and respond to accordingly. Moreover, if the bed 302 is occupied by two users, the control circuitry 334 can learn and respond to the patterns of each of the users.

As described above, the control circuitry 334 for the bed 302 can generate control signals for control functions of various other devices. The control signals can be generated, at least in part, based on detected interactions by the user 308 with the bed 302, as well as other information including time, date, temperature, etc. The control circuitry 334 can communicate with the television 312, receive information from the television 312, and generate control signals for controlling functions of the television 312. For example, the control circuitry 334 can receive an indication from the television 312 that the television 312 is currently turned on. If the television 312 is located in a different room than the bed 302, the control circuitry 334 can generate a control signal to turn the television 312 off upon making a determination that the user 308 has gone to bed for the evening or otherwise is remaining in the room with the bed 302. For example, if presence of the user 308 is detected on the bed 302 during a particular time range (e.g., between 8:00 μm and 7:00 am) and persists for longer than a threshold period of time (e.g., 10 minutes), the control circuitry 334 can determine that the user 308 is in bed for the evening. If the television 312 is on (as indicated by communications received by the control circuitry 334 of the bed 302 from the television 312), the control circuitry 334 can generate a control signal to turn the television 312 off. The control signals can be transmitted to the television (e.g., through a directed communication link between the television 312 and the control circuitry 334 or through a network, such as WIFI). As another example, rather than turning off the television 312 in response to detection of user bed presence, the control circuitry 334 can generate a control signal that causes the volume of the television 312 to be lowered by a pre-specified amount.

As another example, upon detecting that the user 308 has left the bed 302 during a specified time range (e.g., between 6:00 am and 8:00 am), the control circuitry 334 can generate control signals to cause the television 312 to turn on and tune to a pre-specified channel (e.g., the user 308 has indicated a preference for watching the morning news upon getting out of bed). The control circuitry 334 can generate the control signal and transmit the signal to the television 312 to cause the television 312 to turn on and tune to the desired station (which can be stored at the control circuitry 334, the television 312, or another location). As another example, upon detecting that the user 308 has gotten up for the day, the control circuitry 334 can generate and transmit control signals to cause the television 312 to turn on and begin playing a previously recorded program from a digital video recorder (DVR) in communication with the television 312.

As another example, if the television 312 is in the same room as the bed 302, the control circuitry 334 may not cause the television 312 to turn off in response to detection of user bed presence. Rather, the control circuitry 334 can generate and transmit control signals to cause the television 312 to turn off in response to determining that the user 308 is asleep. For example, the control circuitry 334 can monitor biometric signals of the user 308 (e.g., motion, heartrate, respiration rate) to determine that the user 308 has fallen asleep. Upon detecting that the user 308 is sleeping, the control circuitry 334 generates and transmits a control signal to turn the television 312 off. As another example, the control circuitry 334 can generate the control signal to turn off the television 312 after a threshold period of time has passed since the user 308 has fallen asleep (e.g., 10 minutes after the user has fallen asleep). As another example, the control circuitry 334 generates control signals to lower the volume of the television 312 after determining that the user 308 is asleep. As yet another example, the control circuitry 334 generates and transmits a control signal to cause the television to gradually lower in volume over a period of time and then turn off in response to determining that the user 308 is asleep. Any of the control signals described above in reference to the television 312 can also be determined by the central controller previously described.

In some implementations, the control circuitry 334 can similarly interact with other media devices, such as computers, tablets, mobile phones, smart phones, wearable devices, stereo systems, etc. For example, upon detecting that the user 308 is asleep, the control circuitry 334 can generate and transmit a control signal to the user device 310 to cause the user device 310 to turn off, or turn down the volume on a video or audio file being played by the user device 310.

The control circuitry 334 can additionally communicate with the lighting system 314, receive information from the lighting system 314, and generate control signals for controlling functions of the lighting system 314. For example, upon detecting user bed presence on the bed 302 during a certain time frame (e.g., between 8:00 pm and 7:00 am) that lasts for longer than a threshold period of time (e.g., 10 minutes), the control circuitry 334 of the bed 302 can determine that the user 308 is in bed for the evening. In response to this determination, the control circuitry 334 can generate control signals to cause lights in one or more rooms other than the room in which the bed 302 is located to switch off. The control signals can then be transmitted to the lighting system 314 and executed by the lighting system 314 to cause the lights in the indicated rooms to shut off. For example, the control circuitry 334 can generate and transmit control signals to turn off lights in all common rooms, but not in other bedrooms. As another example, the control signals generated by the control circuitry 334 can indicate that lights in all rooms other than the room in which the bed 302 is located are to be turned off, while one or more lights located outside of the house containing the bed 302 are to be turned on, in response to determining that the user 308 is in bed for the evening. Additionally, the control circuitry 334 can generate and transmit control signals to cause the nightlight 328 to turn on in response to determining user 308 bed presence or that the user 308 is asleep. As another example, the control circuitry 334 can generate first control signals for turning off a first set of lights (e.g., lights in common rooms) in response to detecting user bed presence, and second control signals for turning off a second set of lights (e.g., lights in the room in which the bed 302 is located) in response to detecting that the user 308 is asleep.

In some implementations, in response to determining that the user 308 is in bed for the evening, the control circuitry 334 of the bed 302 can generate control signals to cause the lighting system 314 to implement a sunset lighting scheme in the room in which the bed 302 is located. A sunset lighting scheme can include, for example, dimming the lights (either gradually over time, or all at once) in combination with changing the color of the light in the bedroom environment, such as adding an amber hue to the lighting in the bedroom. The sunset lighting scheme can help to put the user 308 to sleep when the control circuitry 334 has determined that the user 308 is in bed for the evening. Sometimes, the control signals can cause the lighting system 314 to dim the lights or change color of the lighting in the bedroom environment, but not both.

The control circuitry 334 can also be configured to implement a sunrise lighting scheme when the user 308 wakes up in the morning. The control circuitry 334 can determine that the user 308 is awake for the day, for example, by detecting that the user 308 has gotten off of the bed 302 (e.g., is no longer present on the bed 302) during a specified time frame (e.g., between 6:00 am and 8:00 am). As another example, the control circuitry 334 can monitor movement, heartrate, respiratory rate, or other biometric signals of the user 308 to determine that the user 308 is awake or is waking up, even though the user 308 has not gotten out of bed. If the control circuitry 334 detects that the user is awake or waking up during a specified timeframe, the control circuitry 334 can determine that the user 308 is awake for the day. The specified timeframe can be, for example, based on previously recorded user bed presence information collected over a period of time (e.g., two weeks) that indicates that the user 308 usually wakes up for the day between 6:30 am and 7:30 am. In response to the control circuitry 334 determining that the user 308 is awake, the control circuitry 334 can generate control signals to cause the lighting system 314 to implement the sunrise lighting scheme in the bedroom in which the bed 302 is located. The sunrise lighting scheme can include, for example, turning on lights (e.g., the lamp 326, or other lights in the bedroom). The sunrise lighting scheme can further include gradually increasing the level of light in the room where the bed 302 is located (or in one or more other rooms). The sunrise lighting scheme can also include only turning on lights of specified colors. For example, the sunrise lighting scheme can include lighting the bedroom with blue light to gently assist the user 308 in waking up and becoming active.

In some implementations, the control circuitry 334 can generate different control signals for controlling actions of one or more components, such as the lighting system 314, depending on a time of day that user interactions with the bed 302 are detected. For example, the control circuitry 334 can use historical user interaction information for interactions between the user 308 and the bed 302 to determine that the user 308 usually falls asleep between 10:00 μm and 11:00 μm and usually wakes up between 6:30 am and 7:30 am on weekdays. The control circuitry 334 can use this information to generate a first set of control signals for controlling the lighting system 314 if the user 308 is detected as getting out of bed at 3:00 am and to generate a second set of control signals for controlling the lighting system 314 if the user 308 is detected as getting out of bed after 6:30 am. For example, if the user 308 gets out of bed prior to 6:30 am, the control circuitry 334 can turn on lights that guide the user 308's route to a bathroom. As another example, if the user 308 gets out of bed prior to 6:30 am, the control circuitry 334 can turn on lights that guide the user 308's route to the kitchen (which can include, for example, turning on the nightlight 328, turning on under bed lighting, turning on the lamp 326, or turning on lights along a path that the user 308 takes to get to the kitchen).

As another example, if the user 308 gets out of bed after 6:30 am, the control circuitry 334 can generate control signals to cause the lighting system 314 to initiate a sunrise lighting scheme, or to turn on one or more lights in the bedroom and/or other rooms. In some implementations, if the user 308 is detected as getting out of bed prior to a specified morning rise time for the user 308, the control circuitry 334 can cause the lighting system 314 to turn on lights that are dimmer than lights that are turned on by the lighting system 314 if the user 308 is detected as getting out of bed after the specified morning rise time. Causing the lighting system 314 to only turn on dim lights when the user 308 gets out of bed during the night (e.g., prior to normal rise time for the user 308) can prevent other occupants of the house from being woken up by the lights while still allowing the user 308 to see in order to reach the bathroom, kitchen, or another destination in the house.

The historical user interaction information for interactions between the user 308 and the bed 302 can be used to identify user sleep and awake timeframes. For example, user bed presence times and sleep times can be determined for a set period of time (e.g., two weeks, a month, etc.). The control circuitry 334 can then identify a typical time range or timeframe in which the user 308 goes to bed, a typical timeframe for when the user 308 falls asleep, and a typical timeframe for when the user 308 wakes up (and in some cases, different timeframes for when the user 308 wakes up and when the user 308 actually gets out of bed). In some implementations, buffer time can be added to these timeframes. For example, if the user is identified as typically going to bed between 10:00 μm and 10:30 pm, a buffer of a half hour in each direction can be added to the timeframe such that any detection of the user getting in bed between 9:30 pm and 11:00 pm is interpreted as the user 308 going to bed for the evening. As another example, detection of bed presence of the user 308 starting from a half hour before the earliest typical time that the user 308 goes to bed extending until the typical wake up time (e.g., 6:30 am) for the user 308 can be interpreted as the user 308 going to bed for the evening. For example, if the user 308 typically goes to bed between 10:00 μm and 10:30 pm, if the user 308's bed presence is sensed at 12:30 am one night, that can be interpreted as the user 308 getting into bed for the evening even though this is outside of the user 308's typical timeframe for going to bed because it has occurred prior to the user 308's normal wake up time. In some implementations, different timeframes are identified for different times of the year (e.g., earlier bed time during winter vs. summer) or at different times of the week (e.g., user 308 wakes up earlier on weekdays than on weekends).

The control circuitry 334 can distinguish between the user 308 going to bed for an extended period (such as for the night) as opposed to being present on the bed 302 for a shorter period (such as for a nap) by sensing duration of presence of the user 308 (e.g., by detecting pressure signals and/or temperature signals of the user 308 on the bed 302 by one or more sensors that are integrated into the bed 302). In some examples, the control circuitry 334 can distinguish between the user 308 going to bed for an extended period (such as for the night) as opposed to going to bed for a shorter period (such as for a nap) by sensing duration of sleep of the user 308. For example, the control circuitry 334 can set a time threshold whereby if the user 308 is sensed on the bed 302 for longer than the threshold, the user 308 is considered to have gone to bed for the night. In some examples, the threshold can be about 2 hours, whereby if the user 308 is sensed on the bed 302 for greater than 2 hours, the control circuitry 334 registers that as an extended sleep event. In other examples, the threshold can be greater than or less than two hours. The threshold can also be determined based on historic trends indicating how long the user 302 usually sleeps or otherwise stays on the bed 302.

The control circuitry 334 can detect repeated extended sleep events to automatically determine a typical bed time range of the user 308, without requiring the user 308 to enter a bed time range. This can allow the control circuitry 334 to accurately estimate when the user 308 is likely to go to bed for an extended sleep event, regardless of whether the user 308 typically goes to bed using a traditional sleep schedule or a non-traditional sleep schedule. The control circuitry 334 can then use knowledge of the bed time range of the user 308 to control one or more components (including components of the bed 302 and/or non-bed peripherals) based on sensing bed presence during the bed time range or outside of the bed time range.

In some examples, the control circuitry 334 can automatically determine the bed time range of the user 308 without requiring user inputs. In some examples, the control circuitry 334 can determine the bed time range of the user 308 automatically and in combination with user inputs (e.g., using one or more signals that are sensed by sensors of the bed 302 and/or the central controller described above). In some examples, the control circuitry 334 can set the bed time range directly according to user inputs. In some examples, the control circuitry 334 can associate different bed times with different days of the week. In each of these examples, the control circuitry 334 can control one or more components (such as the lighting system 314, the thermostat 316, the security system 318, the oven 322, the coffee maker 324, the lamp 326, and the nightlight 328), as a function of sensed bed presence and the bed time range.

The control circuitry 334 can additionally communicate with the thermostat 316, receive information from the thermostat 316, and generate control signals for controlling functions of the thermostat 316. For example, the user 308 can indicate user preferences for different temperatures at different times, depending on the sleep state or bed presence of the user 308. For example, the user 308 may prefer an environmental temperature of 72 degrees when out of bed, 70 degrees when in bed but awake, and 68 degrees when sleeping. The control circuitry 334 of the bed 302 can detect bed presence of the user 308 in the evening and determine that the user 308 is in bed for the night. In response to this determination, the control circuitry 334 can generate control signals to cause the thermostat 316 to change the temperature to 70 degrees. The control circuitry 334 can then transmit the control signals to the thermostat 316. Upon detecting that the user 308 is in bed during the bed time range or asleep, the control circuitry 334 can generate and transmit control signals to cause the thermostat 316 to change the temperature to 68. The next morning, upon determining that the user 308 is awake for the day (e.g., the user 308 gets out of bed after 6:30 am), the control circuitry 334 can generate and transmit control circuitry 334 to cause the thermostat to change the temperature to 72 degrees.

The control circuitry 334 can also determine control signals to be transmitted to the thermostat 316 based on maintaining improved or preferred sleep quality of the user 308. In other words, the control circuitry 334 can determine adjustments to the thermostat 316 that are not merely based on user-inputted preferences. For example, the control circuitry 334 can determine, based on historic sleep patterns and quality of the user 308 and by applying one or more machine learning models, that the user 308 experiences their best sleep when the bedroom is at 74 degrees. The control circuitry 334 can receive temperature signals from one or more devices and/or sensors in the bedroom indicating a temperature of the bedroom. When the temperature is below 74 degrees, the control circuitry 334 can determine control signals that cause the thermostat 316 to activate a heating unit in the house to raise the temperature to 74 degrees in the bedroom. When the temperature is above 74 degrees, the control circuitry 334 can determine control signals that cause the thermostat 316 to activate a cooling unit in the house to lower the temperature back to 74 degrees. Sometimes, the control circuitry 334 can also determine control signals that cause the thermostat 316 to maintain the bedroom within a temperature range that is intended to keep the user 308 in particular sleep states and/or transition to next preferred sleep states.

In some implementations, the control circuitry 334 can generate control signals to cause one or more heating or cooling elements on the surface of the bed 302 to change temperature at various times, either in response to user interaction with the bed 302, at various pre-programmed times, based on user preference, and/or in response to detecting microclimate temperatures of the user 308 on the bed 302. For example, the control circuitry 334 can activate a heating element to raise the temperature of one side of the surface of the bed 302 to 73 degrees when it is detected that the user 308 has fallen asleep. As another example, upon determining that the user 308 is up for the day, the control circuitry 334 can turn off a heating or cooling element. As yet another example, the user 308 can pre-program various times at which the temperature at the surface of the bed should be raised or lowered. For example, the user 308 can program the bed 302 to raise the surface temperature to 76 degrees at 10:00 μm, and lower the surface temperature to 68 degrees at 11:30 pm. As another example, one or more temperature sensors on the surface of the bed 302 can detect microclimates of the user 308 on the bed 302. When a detected microclimate of the user 308 drops below a predetermined threshold temperature, the control circuitry 334 can activate a heating element to raise the user 308's body temperature, thereby improving the user 308's comfortability, maintaining the user 308 in their sleep cycle, transitioning the user 308 to a next preferred sleep state, and/or otherwise maintaining or improving the user 308's sleep quality.

In some implementations, in response to detecting user bed presence of the user 308 and/or that the user 308 is asleep, the control circuitry 334 can cause the thermostat 316 to change the temperature in different rooms to different values. For example, in response to determining that the user 308 is in bed for the evening, the control circuitry 334 can generate and transmit control signals to cause the thermostat 316 to set the temperature in one or more bedrooms of the house to 72 degrees and set the temperature in other rooms to 67 degrees. Other control signals are also possible, and can be based on user preference and user input.

The control circuitry 334 can also receive temperature information from the thermostat 316 and use this temperature information to control functions of the bed 302 or other devices. For example, as discussed above, the control circuitry 334 can adjust temperatures of heating elements included in or otherwise attached to the bed 302 (e.g., a foot warming pad) in response to temperature information received from the thermostat 316.

In some implementations, the control circuitry 334 can generate and transmit control signals for controlling other temperature control systems. For example, in response to determining that the user 308 is awake for the day, the control circuitry 334 can generate and transmit control signals for causing floor heating elements to activate in the bedroom and/or in other rooms in the house. For example, the control circuitry 334 can cause a floor heating system in a master bedroom to turn on in response to determining that the user 308 is awake for the day. One or more of the control signals described herein that are determined by the control circuitry 334 can also be determined by the central controller described above.

The control circuitry 334 can additionally communicate with the security system 318, receive information from the security system 318, and generate control signals for controlling functions of the security system 318. For example, in response to detecting that the user 308 in is bed for the evening, the control circuitry 334 can generate control signals to cause the security system 318 to engage or disengage security functions. The control circuitry 334 can then transmit the control signals to the security system 318 to cause the security system 318 to engage (e.g., turning on security cameras along a perimeter of the house, automatically locking doors in the house, etc.). As another example, the control circuitry 334 can generate and transmit control signals to cause the security system 318 to disable in response to determining that the user 308 is awake for the day (e.g., user 308 is no longer present on the bed 302 after 6:00 am). In some implementations, the control circuitry 334 can generate and transmit a first set of control signals to cause the security system 318 to engage a first set of security features in response to detecting user bed presence of the user 308, and can generate and transmit a second set of control signals to cause the security system 318 to engage a second set of security features in response to detecting that the user 308 has fallen asleep.

In some implementations, the control circuitry 334 can receive alerts from the security system 318 and indicate the alert to the user 308. For example, the control circuitry 334 can detect that the user 308 is in bed for the evening and in response, generate and transmit control signals to cause the security system 318 to engage or disengage. The security system can then detect a security breach (e.g., someone has opened the door 332 without entering the security code, or someone has opened a window when the security system 318 is engaged). The security system 318 can communicate the security breach to the control circuitry 334 of the bed 302. In response to receiving the communication from the security system 318, the control circuitry 334 can generate control signals to alert the user 308 to the security breach. For example, the control circuitry 334 can cause the bed 302 to vibrate. As another example, the control circuitry 334 can cause portions of the bed 302 to articulate (e.g., cause the head section to raise or lower) in order to wake the user 308 and alert the user to the security breach. As another example, the control circuitry 334 can generate and transmit control signals to cause the lamp 326 to flash on and off at regular intervals to alert the user 308 to the security breach. As another example, the control circuitry 334 can alert the user 308 of one bed 302 regarding a security breach in a bedroom of another bed, such as an open window in a kid's bedroom. As another example, the control circuitry 334 can send an alert to a garage door controller (e.g., to close and lock the door). As another example, the control circuitry 334 can send an alert for the security to be disengaged. The control circuitry 334 can also set off a smart alarm or other alarm device/clock near the bed 302. The control circuitry 334 can transmit a push notification, text message, or other indication of the security breach to the user device 310. Also, the control circuitry 334 can transmit a notification of the security breach to the central controller described above The central controller can then determine one or more responses to the security breach.

The control circuitry 334 can additionally generate and transmit control signals for controlling the garage door 320 and receive information indicating a state of the garage door 320 (e.g., open or closed). For example, in response to determining that the user 308 is in bed for the evening, the control circuitry 334 can generate and transmit a request to a garage door opener or another device capable of sensing if the garage door 320 is open. The control circuitry 334 can request information on the current state of the garage door 320. If the control circuitry 334 receives a response (e.g., from the garage door opener) indicating that the garage door 320 is open, the control circuitry 334 can either notify the user 308 that the garage door is open (e.g., by displaying a notification or other message at the user device 310, by outputting a notification at the central controller, etc.), and/or generate a control signal to cause the garage door opener to close the garage door 320. For example, the control circuitry 334 can send a message to the user device 310 indicating that the garage door is open. As another example, the control circuitry 334 can cause the bed 302 to vibrate. As yet another example, the control circuitry 334 can generate and transmit a control signal to cause the lighting system 314 to cause one or more lights in the bedroom to flash to alert the user 308 to check the user device 310 for an alert (in this example, an alert regarding the garage door 320 being open). Alternatively, or additionally, the control circuitry 334 can generate and transmit control signals to cause the garage door opener to close the garage door 320 in response to identifying that the user 308 is in bed for the evening and that the garage door 320 is open. Control signals can also vary depend on the age of the user 308.

The control circuitry 334 can similarly send and receive communications for controlling or receiving state information associated with the door 332 or the oven 322. For example, upon detecting that the user 308 is in bed for the evening, the control circuitry 334 can generate and transmit a request to a device or system for detecting a state of the door 332. Information returned in response to the request can indicate various states of the door 332 such as open, closed but unlocked, or closed and locked. If the door 332 is open or closed but unlocked, the control circuitry 334 can alert the user 308 to the state of the door, such as in a manner described above with reference to the garage door 320. Alternatively, or in addition to alerting the user 308, the control circuitry 334 can generate and transmit control signals to cause the door 332 to lock, or to close and lock. If the door 332 is closed and locked, the control circuitry 334 can determine that no further action is needed.

Similarly, upon detecting that the user 308 is in bed for the evening, the control circuitry 334 can generate and transmit a request to the oven 322 to request a state of the oven 322 (e.g., on or off). If the oven 322 is on, the control circuitry 334 can alert the user 308 and/or generate and transmit control signals to cause the oven 322 to turn off. If the oven is already off, the control circuitry 334 can determine that no further action is necessary. In some implementations, different alerts can be generated for different events. For example, the control circuitry 334 can cause the lamp 326 (or one or more other lights, via the lighting system 314) to flash in a first pattern if the security system 318 has detected a breach, flash in a second pattern if garage door 320 is on, flash in a third pattern if the door 332 is open, flash in a fourth pattern if the oven 322 is on, and flash in a fifth pattern if another bed has detected that a user 308 of that bed has gotten up (e.g., that a child of the user 308 has gotten out of bed in the middle of the night as sensed by a sensor in the child's bed). Other examples of alerts that can be processed by the control circuitry 334 of the bed 302 and communicated to the user (e.g., at the user device 310 and/or the central controller described herein) include a smoke detector detecting smoke (and communicating this detection of smoke to the control circuitry 334), a carbon monoxide tester detecting carbon monoxide, a heater malfunctioning, or an alert from any other device capable of communicating with the control circuitry 334 and detecting an occurrence that should be brought to the user 308's attention.

The control circuitry 334 can also communicate with a system or device for controlling a state of the window blinds 330. For example, in response to determining that the user 308 is in bed for the evening, the control circuitry 334 can generate and transmit control signals to cause the window blinds 330 to close. As another example, in response to determining that the user 308 is up for the day (e.g., user has gotten out of bed after 6:30 am) or that the user 308 set an alarm to wake up at a particular time, the control circuitry 334 can generate and transmit control signals to cause the window blinds 330 to open. By contrast, if the user 308 gets out of bed prior to a normal rise time for the user 308, the control circuitry 334 can determine that the user 308 is not awake for the day and may not generate control signals that cause the window blinds 330 to open. As yet another example, the control circuitry 334 can generate and transmit control signals that cause a first set of blinds to close in response to detecting user bed presence of the user 308 and a second set of blinds to close in response to detecting that the user 308 is asleep.

The control circuitry 334 can generate and transmit control signals for controlling functions of other household devices in response to detecting user interactions with the bed 302. For example, in response to determining that the user 308 is awake for the day, the control circuitry 334 can generate and transmit control signals to the coffee maker 324 to cause the coffee maker 324 to begin brewing coffee. As another example, the control circuitry 334 can generate and transmit control signals to the oven 322 to cause the oven 322 to begin preheating (for users that like fresh baked bread in the morning or otherwise bake or prepare food in the morning). As another example, the control circuitry 334 can use information indicating that the user 308 is awake for the day along with information indicating that the time of year is currently winter and/or that the outside temperature is below a threshold value to generate and transmit control signals to cause a car engine block heater to turn on.

As another example, the control circuitry 334 can generate and transmit control signals to cause one or more devices to enter a sleep mode in response to detecting user bed presence of the user 308, or in response to detecting that the user 308 is asleep. For example, the control circuitry 334 can generate control signals to cause a mobile phone of the user 308 to switch into sleep mode or night mode such that notifications from the mobile phone are muted to not disturb the user 308's sleep. The control circuitry 334 can then transmit the control signals to the mobile phone. Later, upon determining that the user 308 is up for the day, the control circuitry 334 can generate and transmit control signals to cause the mobile phone to switch out of sleep mode.

In some implementations, the control circuitry 334 can communicate with one or more noise control devices. For example, upon determining that the user 308 is in bed for the evening, or that the user 308 is asleep (e.g., based on pressure signals received from the bed 302, audio/decibel signals received from audio sensors positioned on or around the bed 302, etc.), the control circuitry 334 can generate and transmit control signals to cause one or more noise cancelation devices to activate. The noise cancelation devices can, for example, be included as part of the bed 302 or located in the bedroom with the bed 302. As another example, upon determining that the user 308 is in bed for the evening or that the user 308 is asleep, the control circuitry 334 can generate and transmit control signals to turn the volume on, off, up, or down, for one or more sound generating devices, such as a stereo system radio, television, computer, tablet, mobile phone, etc.

Additionally, functions of the bed 302 can be controlled by the control circuitry 334 in response to user interactions with the bed 302. As mentioned throughout, functions of the bed 302 described herein can also be controlled by the user device 310 and/or the central controller (e.g., a hub device or other home automation device that controls multiple different devices in the home). As mentioned above, the bed 302 can include an adjustable foundation and an articulation controller configured to adjust the position of one or more portions of the bed 302 by adjusting the adjustable foundation that supports the bed 302. For example, the articulation controller can adjust the bed 302 from a flat position to a position in which a head portion of a mattress of the bed 302 is inclined upward (e.g., to facilitate a user sitting up in bed, reading, and/or watching television). In some implementations, the bed 302 includes multiple separately articulable sections. For example, portions of the bed corresponding to the locations of the air chambers 306a and 306b can be articulated independently from each other, to allow one person positioned on the bed 302 surface to rest in a first position (e.g., a flat position) while a second person rests in a second position (e.g., a reclining position with the head raised at an angle from the waist). In some implementations, separate positions can be set for two different beds (e.g., two twin beds placed next to each other). The foundation of the bed 302 can include more than one zone that can be independently adjusted. The articulation controller can also be configured to provide different levels of massage to one or more users on the bed 302 or to cause the bed to vibrate to communicate alerts to the user 308 as described above.

The control circuitry 334 can adjust positions (e.g., incline and decline positions for the user 308 and/or an additional user of the bed 302) in response to user interactions with the bed 302. For example, the control circuitry 334 can cause the articulation controller to adjust the bed 302 to a first recline position for the user 308 in response to sensing user bed presence for the user 308. The control circuitry 334 can cause the articulation controller to adjust the bed 302 to a second recline position (e.g., a less reclined, or flat position) in response to determining that the user 308 is asleep. As another example, the control circuitry 334 can receive a communication from the television 312 indicating that the user 308 has turned off the television 312, and in response, the control circuitry 334 can cause the articulation controller to adjust the position of the bed 302 to a preferred user sleeping position (e.g., due to the user turning off the television 312 while the user 308 is in bed indicating that the user 308 wishes to go to sleep).

In some implementations, the control circuitry 334 can control the articulation controller so as to wake up one user of the bed 302 without waking another user of the bed 302. For example, the user 308 and a second user of the bed 302 can each set distinct wakeup times (e.g., 6:30 am and 7:15 am respectively). When the wakeup time for the user 308 is reached, the control circuitry 334 can cause the articulation controller to vibrate or change the position of only a side of the bed on which the user 308 is located to wake the user 308 without disturbing the second user. When the wakeup time for the second user is reached, the control circuitry 334 can cause the articulation controller to vibrate or change the position of only the side of the bed on which the second user is located. Alternatively, when the second wakeup time occurs, the control circuitry 334 can utilize other methods (such as audio alarms, or turning on the lights) to wake the second user since the user 308 is already awake and therefore will not be disturbed when the control circuitry 334 attempts to wake the second user.

Still referring to FIG. 3, the control circuitry 334 for the bed 302 can utilize information for interactions with the bed 302 by multiple users to generate control signals for controlling functions of various other devices. For example, the control circuitry 334 can wait to generate control signals for, for example, engaging the security system 318, or instructing the lighting system 314 to turn off lights in various rooms, until both the user 308 and a second user are detected as being present on the bed 302. As another example, the control circuitry 334 can generate a first set of control signals to cause the lighting system 314 to turn off a first set of lights upon detecting bed presence of the user 308 and generate a second set of control signals for turning off a second set of lights in response to detecting bed presence of a second user. As another example, the control circuitry 334 can wait until it has been determined that both the user 308 and a second user are awake for the day before generating control signals to open the window blinds 330. As yet another example, in response to determining that the user 308 has left the bed 302 and is awake for the day, but that a second user is still sleeping, the control circuitry 334 can generate and transmit a first set of control signals to cause the coffee maker 324 to begin brewing coffee, to cause the security system 318 to deactivate, to turn on the lamp 326, to turn off the nightlight 328, to cause the thermostat 316 to raise the temperature in one or more rooms to 72 degrees, and/or to open the window blinds 330 in rooms other than the bedroom in which the bed 302 is located. Later, in response to detecting that the second user is no longer present on the bed (or that the second user is awake or is waking up) the control circuitry 334 can generate and transmit a second set of control signals to, for example, cause the lighting system 314 to turn on one or more lights in the bedroom, to cause window blinds in the bedroom to open, and to turn on the television 312 to a pre-specified channel. One or more other home automation control signals can be determined and generated by the control circuitry 334, the user device 310, and/or the central controller described herein.

### Examples of Data Processing Systems Associated with a Bed

Described here are examples of systems and components that can be used for data processing tasks that are, for example, associated with a bed. In some cases, multiple examples of a particular component or group of components are presented. Some of these examples are redundant and/or mutually exclusive alternatives. Connections between components are shown as examples to illustrate possible network configurations for allowing communication between components. Different formats of connections can be used as technically needed or desired. The connections generally indicate a logical connection that can be created with any technologically feasible format. For example, a network on a motherboard can be created with a printed circuit board, wireless data connections, and/or other types of network connections. Some logical connections are not shown for clarity. For example, connections with power supplies and/or computer readable memory may not be shown for clarities sake, as many or all elements of a particular component may need to be connected to the power supplies and/or computer readable memory.

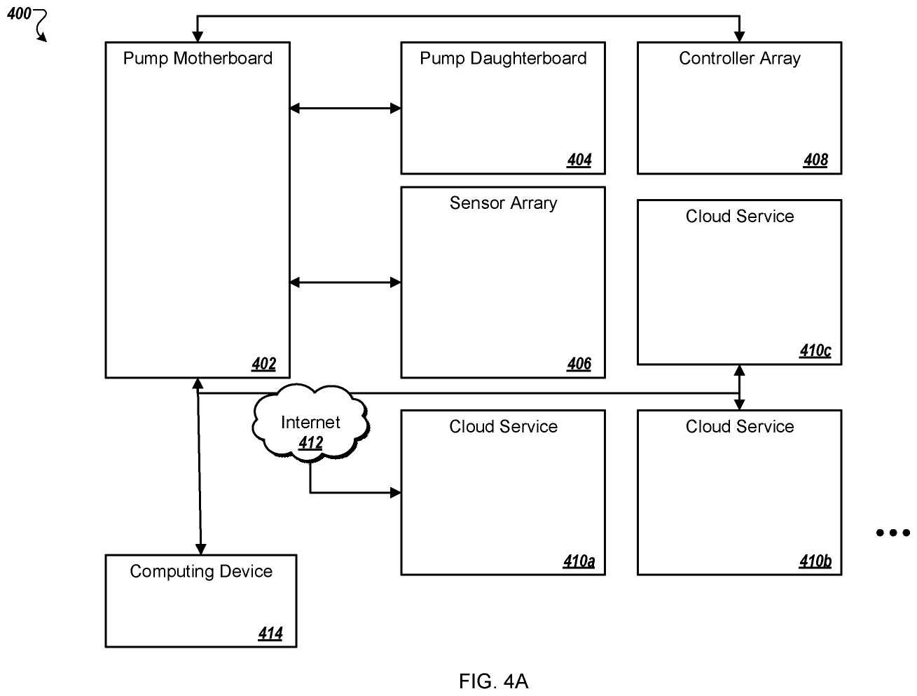

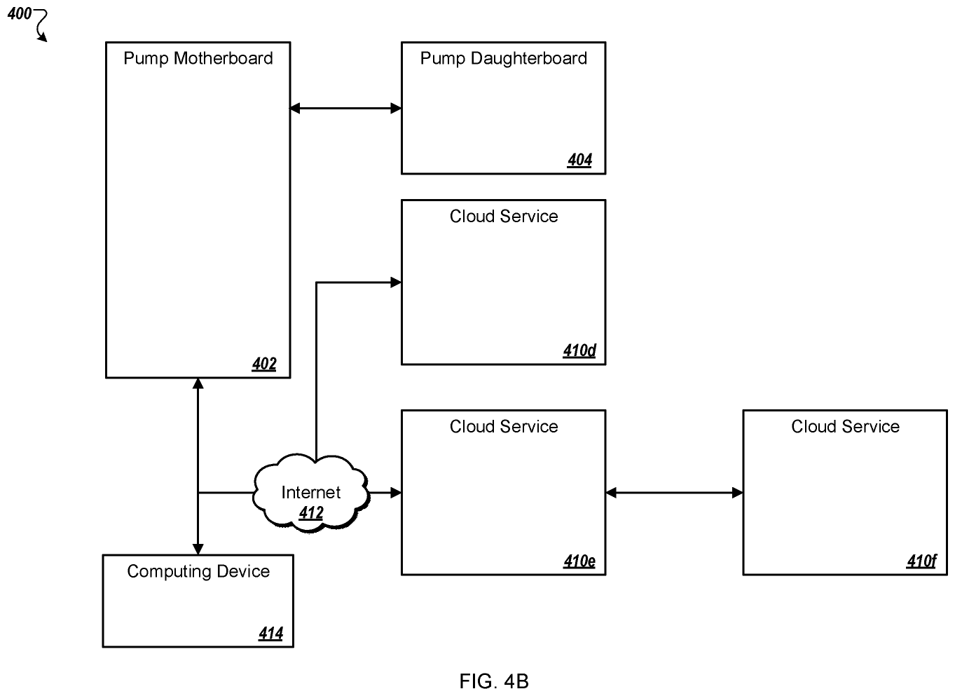

FIG. 4A is a block diagram of an example of a data processing system 400 that can be associated with a bed system, including those described above with respect to FIGS. 1-3. This system 400 includes a pump motherboard 402 and a pump daughterboard 404. The system 400 includes a sensor array 406 that can include one or more sensors configured to sense physical phenomenon of the environment and/or bed, and to report such sensing back to the pump motherboard 402 for, for example, analysis. The sensor array 406 can include one or more different types of sensors, including but not limited to pressure sensors, temperature sensors, light sensors, movement (e.g. motion) sensors, and audio sensors. The system 400 also includes a controller array 408 that can include one or more controllers configured to control logic-controlled devices of the bed and/or environment (such as home automation devices, security systems light systems, and other devices that are described in reference to FIG. 3). The pump motherboard 400 can be in communication with one or more computing devices 414 and one or more cloud services 410 over local networks, the Internet 412, or otherwise as is technically appropriate. Each of these components will be described in more detail, some with multiple example configurations, below.

In this example, a pump motherboard 402 and a pump daughterboard 404 are communicably coupled. They can be conceptually described as a center or hub of the system 400, with the other components conceptually described as spokes of the system 400. In some configurations, this can mean that each of the spoke components communicates primarily or exclusively with the pump motherboard 402. For example, a sensor of the sensor array 406 may not be configured to, or may not be able to, communicate directly with a corresponding controller. Instead, each spoke component can communicate with the motherboard 402. The sensor of the sensor array 406 can report a sensor reading to the motherboard 402, and the motherboard 402 can determine that, in response, a controller of the controller array 408 should adjust some parameters of a logic controlled device or otherwise modify a state of one or more peripheral devices. In one case, if the temperature of the bed is determined to be too hot based on received temperature signals from the sensor array 406, the pump motherboard 402 can determine that a temperature controller should cool the bed.

One advantage of a hub-and-spoke network configuration, sometimes also referred to as a star-shaped network, is a reduction in network traffic compared to, for example, a mesh network with dynamic routing. If a particular sensor generates a large, continuous stream of traffic, that traffic may only be transmitted over one spoke of the network to the motherboard 402. The motherboard 402 can, for example, marshal that data and condense it to a smaller data format for retransmission for storage in a cloud service 410. Additionally or alternatively, the motherboard 402 can generate a single, small, command message to be sent down a different spoke of the network in response to the large stream. For example, if the large stream of data is a pressure reading that is transmitted from the sensor array 406 a few times a second, the motherboard 402 can respond with a single command message to the controller array to increase the pressure in an air chamber of the bed. In this case, the single command message can be orders of magnitude smaller than the stream of pressure readings.

As another advantage, a hub-and-spoke network configuration can allow for an extensible network that can accommodate components being added, removed, failing, etc. This can allow, for example, more, fewer, or different sensors in the sensor array 406, controllers in the controller array 408, computing devices 414, and/or cloud services 410. For example, if a particular sensor fails or is deprecated by a newer version of the sensor, the system 400 can be configured such that only the motherboard 402 needs to be updated about the replacement sensor. This can allow, for example, product differentiation where the same motherboard 402 can support an entry level product with fewer sensors and controllers, a higher value product with more sensors and controllers, and customer personalization where a customer can add their own selected components to the system 400.

Additionally, a line of air bed products can use the system 400 with different components. In an application in which every air bed in the product line includes both a central logic unit and a pump, the motherboard 402 (and optionally the daughterboard 404) can be designed to fit within a single, universal housing. Then, for each upgrade of the product in the product line, additional sensors, controllers, cloud services, etc., can be added. Design, manufacturing, and testing time can be reduced by designing all products in a product line from this base, compared to a product line in which each product has a bespoke logic control system.

Each of the components discussed above can be realized in a wide variety of technologies and configurations. Below, some examples of each component will be further discussed. In some alternatives, two or more of the components of the system 400 can be realized in a single alternative component; some components can be realized in multiple, separate components; and/or some functionality can be provided by different components.

FIG. 4B is a block diagram showing some communication paths of the data processing system 400. As previously described, the motherboard 402 and the pump daughterboard 404 may act as a hub for peripheral devices and cloud services of the system 400. In cases in which the pump daughterboard 404 communicates with cloud services or other components, communications from the pump daughterboard 404 may be routed through the pump motherboard 402. This may allow, for example, the bed to have only a single connection with the internet 412. The computing device 414 may also have a connection to the internet 412, possibly through the same gateway used by the bed and/or possibly through a different gateway (e.g., a cell service provider).

Previously, a number of cloud services 410 were described. As shown in FIG. 4B, some cloud services, such as cloud services 410d and 410e, may be configured such that the pump motherboard 402 can communicate with the cloud service directly—that is the motherboard 402 may communicate with a cloud service 410 without having to use another cloud service 410 as an intermediary. Additionally or alternatively, some cloud services 410, for example cloud service 410f, may only be reachable by the pump motherboard 402 through an intermediary cloud service, for example cloud service 410e. While not shown here, some cloud services 410 may be reachable either directly or indirectly by the pump motherboard 402.

Additionally, some or all of the cloud services 410 may be configured to communicate with other cloud services. This communication may include the transfer of data and/or remote function calls according to any technologically appropriate format. For example, one cloud service 410 may request a copy for another cloud service's 410 data, for example, for purposes of backup, coordination, migration, or for performance of calculations or data mining. In another example, many cloud services 410 may contain data that is indexed according to specific users tracked by the user account cloud 410c and/or the bed data cloud 410a. These cloud services 410 may communicate with the user account cloud 410c and/or the bed data cloud 410a when accessing data specific to a particular user or bed.

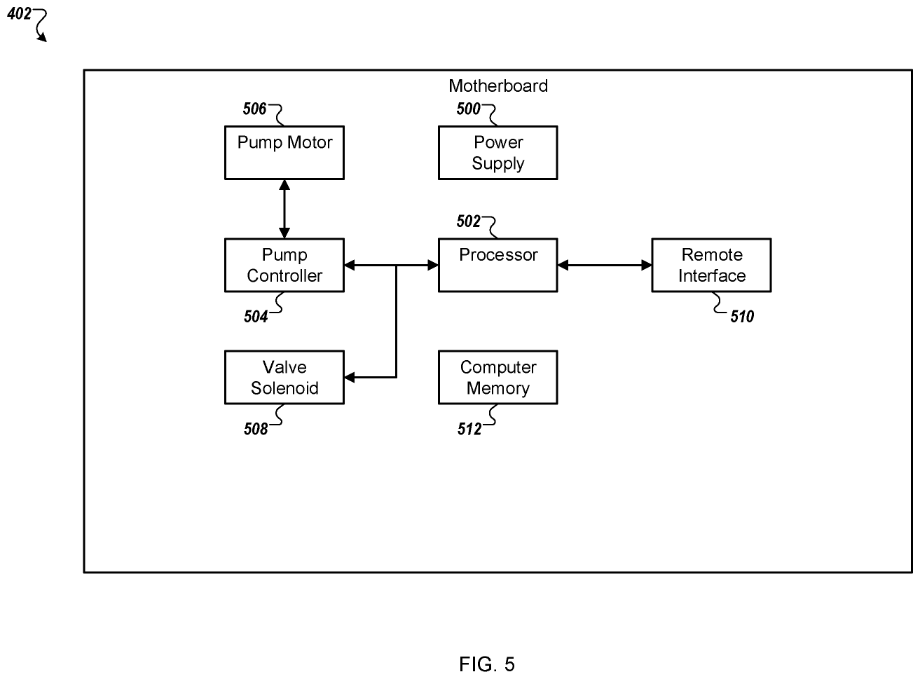

FIG. 5 is a block diagram of an example of a motherboard 402 that can be used in a data processing system that can be associated with a bed system, including those described above with respect to FIGS. 1-3. In this example, compared to other examples described below, this motherboard 402 consists of relatively fewer parts and can be limited to provide a relatively limited feature set.

The motherboard 402 includes a power supply 500, a processor 502, and computer memory 512. In general, the power supply 500 includes hardware used to receive electrical power from an outside source and supply it to components of the motherboard 402. The power supply can include, for example, a battery pack and/or wall outlet adapter, an AC to DC converter, a DC to AC converter, a power conditioner, a capacitor bank, and/or one or more interfaces for providing power in the current type, voltage, etc., needed by other components of the motherboard 402.

The processor 502 is generally a device for receiving input, performing logical determinations, and providing output. The processor 502 can be a central processing unit, a microprocessor, general purpose logic circuitry, application-specific integrated circuitry, a combination of these, and/or other hardware for performing the functionality needed.

The memory 512 is generally one or more devices for storing data. The memory 512 can include long term stable data storage (e.g., on a hard disk), short term unstable (e.g., on Random Access Memory) or any other technologically appropriate configuration.

The motherboard 402 includes a pump controller 504 and a pump motor 506. The pump controller 504 can receive commands from the processor 502 and, in response, control the functioning of the pump motor 506. For example, the pump controller 504 can receive, from the processor 502, a command to increase pressure of an air chamber by 0.3 pounds per square inch (PSI). The pump controller 504, in response, engages a valve so that the pump motor 506 is configured to pump air into the selected air chamber, and can engage the pump motor 506 for a length of time that corresponds to 0.3 PSI or until a sensor indicates that pressure has been increased by 0.3 PSI. In an alternative configuration, the message can specify that the chamber should be inflated to a target PSI, and the pump controller 504 can engage the pump motor 506 until the target PSI is reached.

A valve solenoid 508 can control which air chamber a pump is connected to. In some cases, the solenoid 508 can be controlled by the processor 502 directly. In some cases, the solenoid 508 can be controlled by the pump controller 504.

A remote interface 510 of the motherboard 402 can allow the motherboard 402 to communicate with other components of a data processing system. For example, the motherboard 402 can be able to communicate with one or more daughterboards, with peripheral sensors, and/or with peripheral controllers through the remote interface 510. The remote interface 510 can provide any technologically appropriate communication interface, including but not limited to multiple communication interfaces such as WIFI, Bluetooth, and copper wired networks.

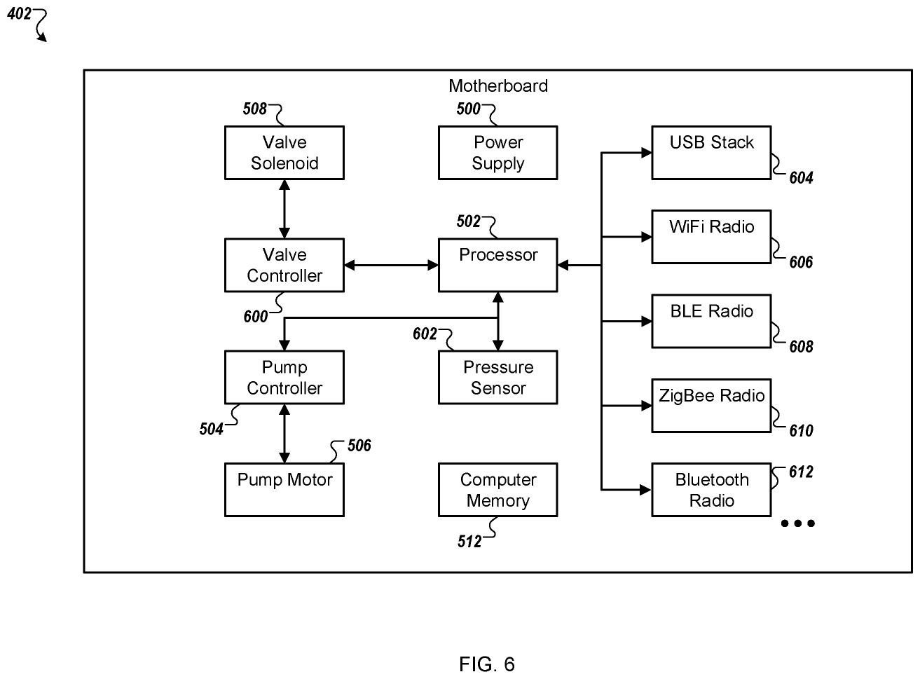

FIG. 6 is a block diagram of an example of the motherboard 402 that can be used in a data processing system associated with a bed system, including those described above with respect to FIGS. 1-3. Compared to the motherboard 402 described with reference to FIG. 5, the motherboard 402 in FIG. 6 can contain more components and provide more functionality in some applications.

In addition to the power supply 500, processor 502, pump controller 504, pump motor 506, and valve solenoid 508, this motherboard 402 is shown with a valve controller 600, a pressure sensor 602, a universal serial bus (USB) stack 604, a WiFi radio 606, a Bluetooth Low Energy (BLE) radio 608, a ZigBee radio 610, a Bluetooth radio 612, and a computer memory 512.

Similar to the way that the pump controller 504 converts commands from the processor 502 into control signals for the pump motor 506, the valve controller 600 can convert commands from the processor 502 into control signals for the valve solenoid 508. In one example, the processor 502 can issue a command to the valve controller 600 to connect the pump to a particular air chamber out of a group of air chambers in an air bed. The valve controller 600 can control the position of the valve solenoid 508 so that the pump is connected to the indicated air chamber.

The pressure sensor 602 can read pressure readings from one or more air chambers of the air bed. The pressure sensor 602 can also preform digital sensor conditioning. As described herein, multiple pressure sensors 602 can be included as part of the motherboard 402 or otherwise in communication with the motherboard 402.

The motherboard 402 can include a suite of network interfaces 604, 606, 608, 610, 612, etc., including but not limited to those shown in FIG. 6. These network interfaces can allow the motherboard to communicate over a wired or wireless network with any number of devices, including but not limited to peripheral sensors, peripheral controllers, computing devices, and devices and services connected to the Internet 412.

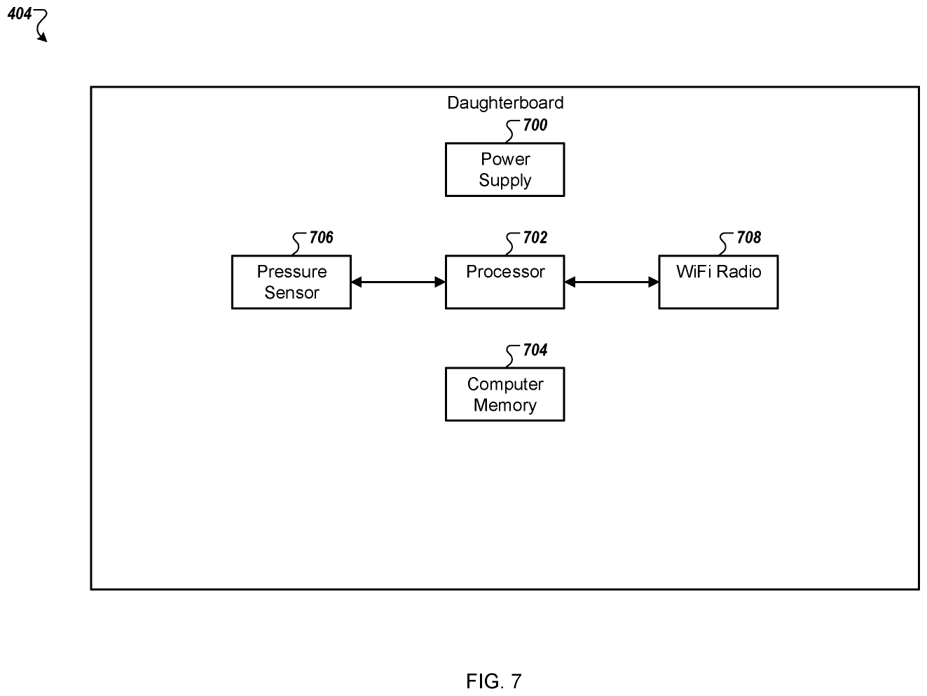

FIG. 7 is a block diagram of an example of a daughterboard 404 that can be used in a data processing system associated with a bed system, including those described above with respect to FIGS. 1-3. In some configurations, one or more daughterboards 404 can be connected to the motherboard 402. Some daughterboards 404 can be designed to offload particular and/or compartmentalized tasks from the motherboard 402. This can be advantageous, for example, if the particular tasks are computationally intensive, proprietary, or subject to future revisions. For example, the daughterboard 404 can be used to calculate a particular sleep data metric. This metric can be computationally intensive, and calculating the sleep metric on the daughterboard 404 can free up the resources of the motherboard 402 while the metric is being calculated. Additionally and/or alternatively, the sleep metric can be subject to future revisions. To update the system 400 with the new sleep metric, it is possible that only the daughterboard 404 that calculates that metric need be replaced. In this case, the same motherboard 402 and other components can be used, saving the need to perform unit testing of additional components instead of just the daughterboard 404.

The daughterboard 404 is shown with a power supply 700, a processor 702, computer readable memory 704, a pressure sensor 706, and a WiFi radio 708. The processor 702 can use the pressure sensor 706 to gather information about the pressure of an air chamber or chambers of an air bed. From this data, the processor 702 can perform an algorithm to calculate a sleep metric (e.g., sleep quality, whether a user is presently in the bed, whether the user has fallen asleep, a heartrate of the user, a respiration rate of the user, movement of the user, etc.). In some examples, the sleep metric can be calculated from only the pressure of air chambers. In other examples, the sleep metric can be calculated using signals from a variety of sensors (e.g., a movement sensor, a pressure sensor, a temperature sensor, and/or an audio sensor). In an example in which different data is needed, the processor 702 can receive that data from an appropriate sensor or sensors. These sensors can be internal to the daughterboard 404, accessible via the WiFi radio 708, or otherwise in communication with the processor 702. Once the sleep metric is calculated, the processor 702 can report that sleep metric to, for example, the motherboard 402. The motherboard 402 can then generate instructions for outputting the sleep metric to the user or otherwise using the sleep metric to determine one or more other information about the user or controls to control the bed system and/or peripheral devices.

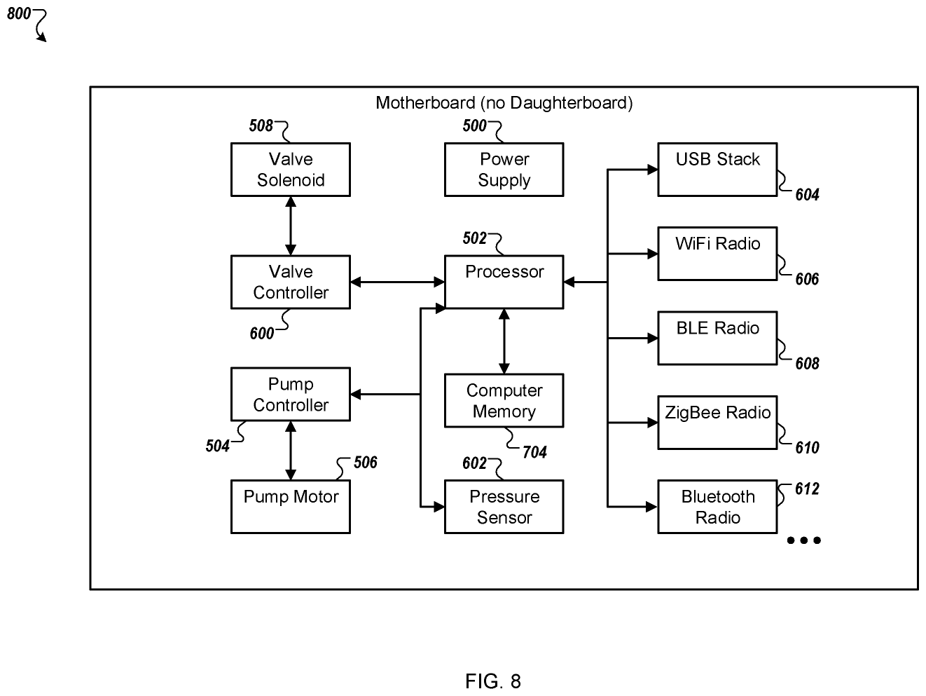

FIG. 8 is a block diagram of an example of a motherboard 800 with no daughterboard that can be used in a data processing system associated with a bed system, including those described above with respect to FIGS. 1-3. In this example, the motherboard 800 can perform most, all, or more of the features described with reference to the motherboard 402 in FIG. 6 and the daughterboard 404 in FIG. 7.

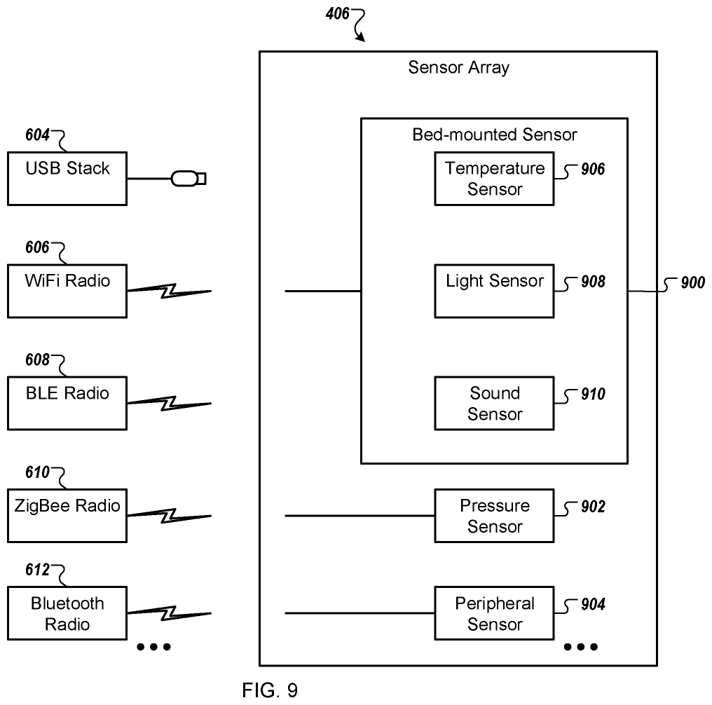

FIG. 9 is a block diagram of an example of the sensory array 406 that can be used in a data processing system associated with a bed system, including those described above with respect to FIGS. 1-3. In general, the sensor array 406 is a conceptual grouping of some or all the peripheral sensors that communicate with the motherboard 402 but are not native to the motherboard 402.

The peripheral sensors 902, 904, 906, 908, 910, etc. of the sensor array 406 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a sensor that outputs a reading over a USB cable can communicate through the USB stack 604.

Some of the peripheral sensors of the sensor array 406 can be bed mounted sensors 900, such as a temperature sensor 906, a light sensor 908, and a sound sensor 910. The bed mounted sensors 900 can be, for example, embedded into the structure of a bed and sold with the bed, or later affixed to the structure of the bed (e.g., part of a pressure sensing pad that is removably installed on a top surface of the bed, part of a temperature sensing or heating pad that is removably installed on the top surface of the bed, integrated into the top surface of the bed, attached along connecting tubes between a pump and air chambers, within air chambers, attached to a headboard of the bed, attached to one or more regions of an adjustable foundation, etc.). Other sensors 902 and 904 can be in communication with the motherboard 402, but optionally not mounted to the bed. The other sensors 902 and 904 can include a pressure sensor 902 and/or peripheral sensor 904. For example, the sensors 902 and 904 can be integrated or otherwise part of a user mobile device (e.g., mobile phone, wearable device, etc.). The sensors 902 and 904 can also be part of a central controller for controlling the bed and peripheral devices in the home. Sometimes, the sensors 902 and 904 can also be part of one or more home automation devices or other peripheral devices in the home.

In some cases, some or all of the bed mounted sensors 900 and/or sensors 902 and 904 can share networking hardware, including a conduit that contains wires from each sensor, a multi-wire cable or plug that, when affixed to the motherboard 402, connect all of the associated sensors with the motherboard 402. In some embodiments, one, some, or all of sensors 902, 904, 906, 908, and 910 can sense one or more features of a mattress, such as pressure, temperature, light, sound, and/or one or more other features of the mattress. In some embodiments, one, some, or all of sensors 902, 904, 906, 908, and 910 can sense one or more features external to the mattress. In some embodiments, pressure sensor 902 can sense pressure of the mattress while some or all of sensors 902, 904, 906, 908, and 910 can sense one or more features of the mattress and/or external to the mattress.

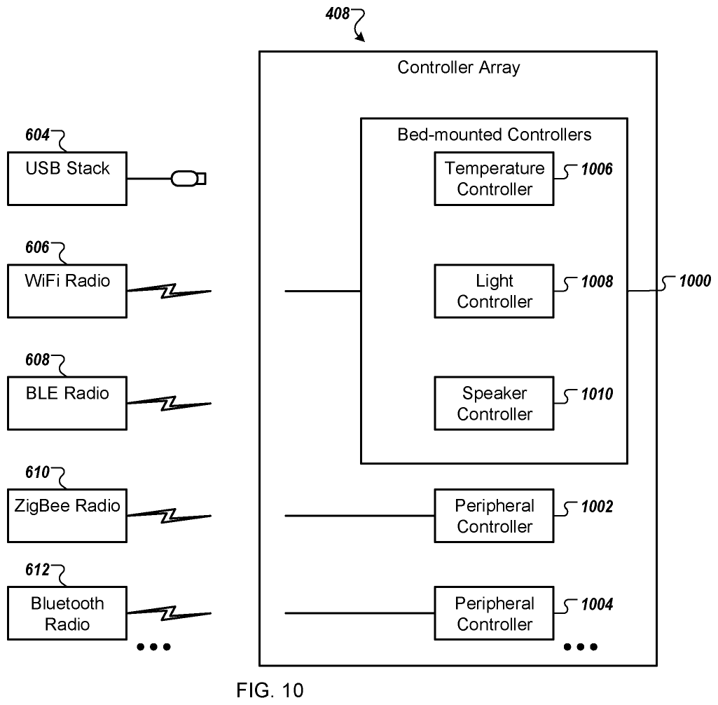

FIG. 10 is a block diagram of an example of the controller array 408 that can be used in a data processing system associated with a bed system, including those described above with respect to FIGS. 1-3. In general, the controller array 408 is a conceptual grouping of some or all peripheral controllers that communicate with the motherboard 402 but are not native to the motherboard 402.

The peripheral controllers of the controller array 408 can communicate with the motherboard 402 through one or more of the network interfaces of the motherboard, including but not limited to the USB stack 604, WiFi radio 606, Bluetooth Low Energy (BLE) radio 608, ZigBee radio 610, and Bluetooth radio 612, as is appropriate for the configuration of the particular sensor. For example, a controller that receives a command over a USB cable can communicate through the USB stack 604.

Some of the controllers of the controller array 408 can be bed mounted controllers 1000, such as a temperature controller 1006, a light controller 1008, and a speaker controller 1010. The bed mounting controllers 1000 can be, for example, embedded into the structure of a bed and sold with the bed, or later affixed to the structure of the bed, as described in reference to the peripheral sensors in FIG. 9. Other peripheral controllers 1002 and 1004 can be in communication with the motherboard 402, but optionally not mounted to the bed. In some cases, some or all of the bed mounted controllers 1000 and/or the peripheral controllers 1002 and 1004 can share networking hardware, including a conduit that contains wires for each controller, a multi-wire cable or plug that, when affixed to the motherboard 402, connects all of the associated controllers with the motherboard 402.

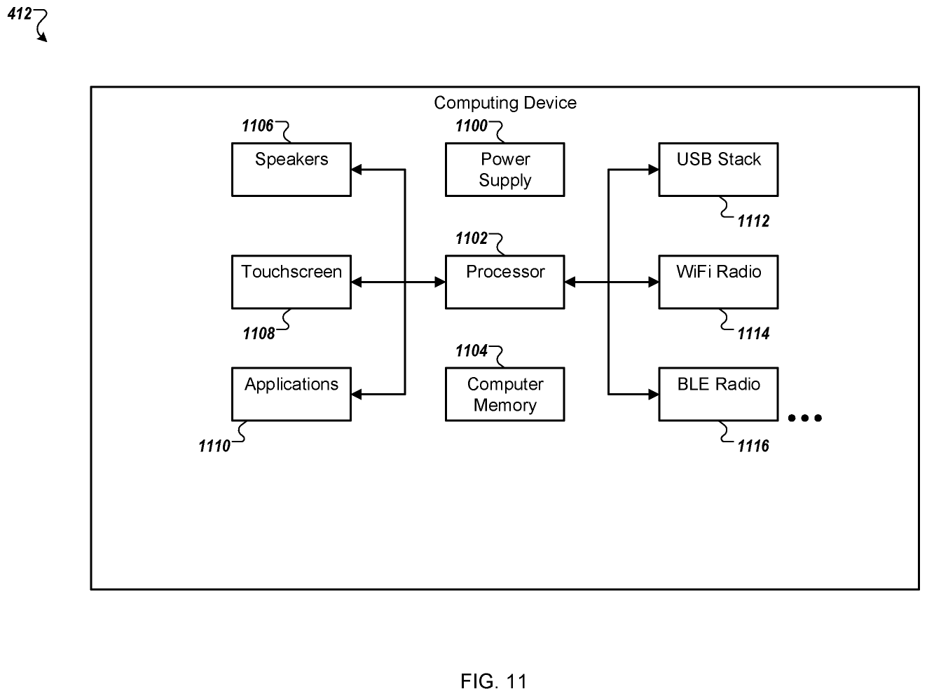

FIG. 11 is a block diagram of an example of the computing device 412 that can be used in a data processing system associated with a bed system, including those described above with respect to FIGS. 1-3. The computing device 412 can include, for example, computing devices used by a user of a bed. Example computing devices 412 include, but are not limited to, mobile computing devices (e.g., mobile phones, tablet computers, laptops, smart phones, wearable devices), desktop computers, home automation devices, and/or central controllers or other hub devices.

The computing device 412 includes a power supply 1100, a processor 1102, and computer readable memory 1104. User input and output can be transmitted by, for example, speakers 1106, a touchscreen 1108, or other not shown components, such as a pointing device or keyboard. The computing device 412 can run one or more applications 1110. These applications can include, for example, applications to allow the user to interact with the system 400. These applications can allow a user to view information about the bed (e.g., sensor readings, sleep metrics), information about themselves (e.g., health conditions that are detected based on signals that are sensed at the bed), and/or configure the behavior of the system 400 (e.g., set a desired firmness to the bed, set desired behavior for peripheral devices). In some cases, the computing device 412 can be used in addition to, or to replace, the remote control 122 described previously.

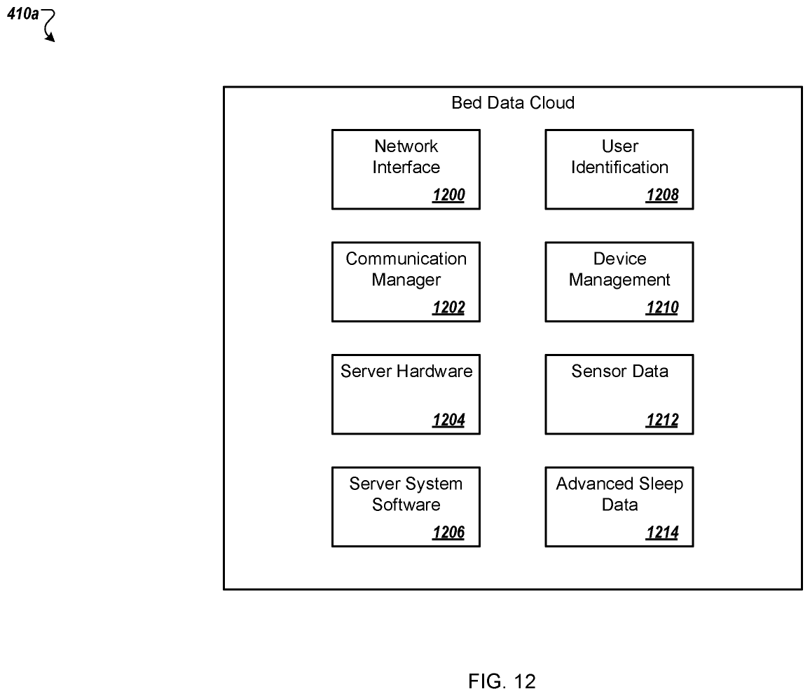

FIG. 12 is a block diagram of an example bed data cloud service 410a that can be used in a data processing system associated with a bed system, including those described above with respect to FIGS. 1-3. In this example, the bed data cloud service 410a is configured to collect sensor data and sleep data from a particular bed, and to match the sensor and sleep data with one or more users that use the bed when the sensor and sleep data was generated.

The bed data cloud service 410a is shown with a network interface 1200, a communication manager 1202, server hardware 1204, and server system software 1206. In addition, the bed data cloud service 410a is shown with a user identification module 1208, a device management 1210 module, a sensor data module 1210, and an advanced sleep data module 1214.

The network interface 1200 generally includes hardware and low level software used to allow one or more hardware devices to communicate over networks. For example the network interface 1200 can include network cards, routers, modems, and other hardware needed to allow the components of the bed data cloud service 410a to communicate with each other and other destinations over, for example, the Internet 412.

The communication manager 1202 generally comprises hardware and software that operate above the network interface 1200. This includes software to initiate, maintain, and tear down network communications used by the bed data cloud service 410a. This includes, for example, TCP/IP, SSL or TLS, Torrent, and other communication sessions over local or wide area networks. The communication manager 1202 can also provide load balancing and other services to other elements of the bed data cloud service 410a.

The server hardware 1204 generally includes physical processing devices used to instantiate and maintain the bed data cloud service 410a. This hardware includes, but is not limited to, processors (e.g., central processing units, ASICs, graphical processers) and computer readable memory (e.g., random access memory, stable hard disks, tape backup). One or more servers can be configured into clusters, multi-computer, or datacenters that can be geographically separate or connected.

The server system software 1206 generally includes software that runs on the server hardware 1204 to provide operating environments to applications and services. The server system software 1206 can include operating systems running on real servers, virtual machines instantiated on real servers to create many virtual servers, server level operations such as data migration, redundancy, and backup.

The user identification 1208 can include, or reference, data related to users of beds with associated data processing systems. For example, the users can include customers, owners, or other users registered with the bed data cloud service 410a or another service. Each user can have, for example, a unique identifier, user credentials, contact information, billing information, demographic information, or any other technologically appropriate information.

The device manager 1210 can include, or reference, data related to beds or other products associated with data processing systems. For example, the beds can include products sold or registered with a system associated with the bed data cloud service 410a. Each bed can have, for example, a unique identifier, model and/or serial number, sales information, geographic information, delivery information, a listing of associated sensors and control peripherals, etc. Additionally, an index or indexes stored by the bed data cloud service 410a can identify users that are associated with beds. For example, this index can record sales of a bed to a user, users that sleep in a bed, etc.

The sensor data 1212 can record raw or condensed sensor data recorded by beds with associated data processing systems. For example, a bed's data processing system can have a temperature sensor, pressure sensor, motion sensor, audio sensor, and/or light sensor. Readings from one or more of these sensors, either in raw form or in a format generated from the raw data (e.g. sleep metrics) of the sensors, can be communicated by the bed's data processing system to the bed data cloud service 410a for storage in the sensor data 1212. Additionally, an index or indexes stored by the bed data cloud service 410a can identify users and/or beds that are associated with the sensor data 1212.

The bed data cloud service 410a can use any of its available data, such as the sensor data 1212, to generate advanced sleep data 1214. In general, the advanced sleep data 1214 includes sleep metrics and other data generated from sensor readings, such as health information associated with the user of a particular bed. Some of these calculations can be performed in the bed data cloud service 410a instead of locally on the bed's data processing system, for example, because the calculations can be computationally complex or require a large amount of memory space or processor power that may not be available on the bed's data processing system. This can help allow a bed system to operate with a relatively simple controller and still be part of a system that performs relatively complex tasks and computations.

For example, the bed data cloud service 410a can retrieve one or more machine learning models from a remote data store and use those models to determine the advanced sleep data 1214. The bed data cloud service 410a can retrieve different types of models based on a type of the advanced sleep data 1214 that is being generated. As an illustrative example, the bed data cloud service 410a can retrieve one or more models to determine overall sleep quality of the user based on currently detected sensor data 1212 and/or historic sensor data (e.g., which can be stored in and accessed from a data store). The bed data cloud service 410a can retrieve one or more other models to determine whether the user is currently snoring based on the detected sensor data 1212. The bed data cloud service 410a can also retrieve one or more other models that can be used to determine whether the user is experiencing some health condition based on the detected sensor data 1212.

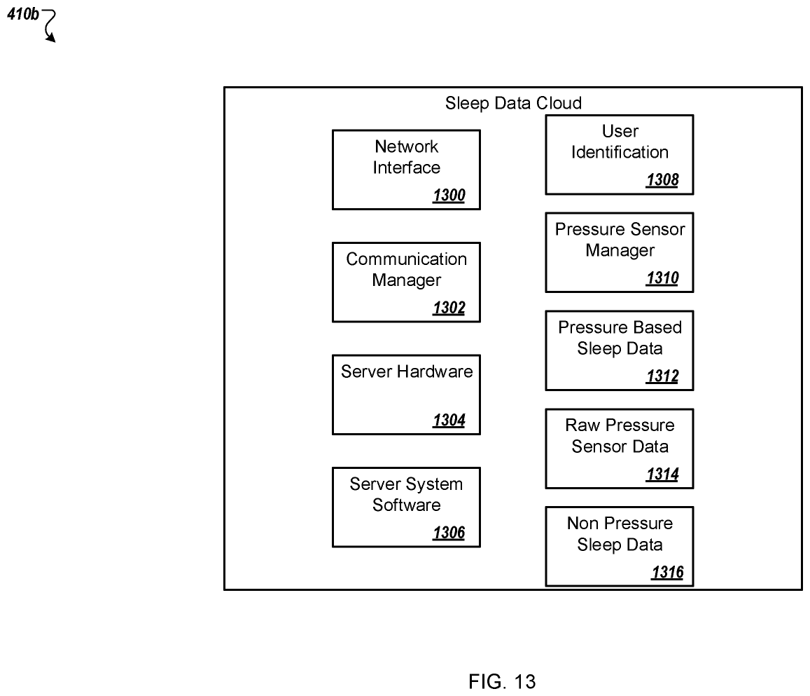

FIG. 13 is a block diagram of an example sleep data cloud service 410b that can be used in a data processing system associated with a bed system, including those described above with respect to FIGS. 1-3. In this example, the sleep data cloud service 410b is configured to record data related to users' sleep experience.

The sleep data cloud service 410b is shown with a network interface 1300, a communication manager 1302, server hardware 1304, and server system software 1306. In addition, the sleep data cloud service 410b is shown with a user identification module 1308, a pressure sensor manager 1310, a pressure based sleep data module 1312, a raw pressure sensor data module 1314, and a non-pressure sleep data module 1316. Sometimes, the sleep data cloud service 410b can include a sensor manager for each of the sensors that are integrated or otherwise in communication with the bed. In some implementations, the sleep data cloud service 410b can include a sensor manager that relates to multiple sensors in beds. For example, a single sensor manager can relate to pressure, temperature, light, movement, and audio sensors in a bed.

Referring to the sleep data cloud service 410b in FIG. 13, the pressure sensor manager 1310 can include, or reference, data related to the configuration and operation of pressure sensors in beds. For example, this data can include an identifier of the types of sensors in a particular bed, their settings and calibration data, etc.

The pressure based sleep data 1312 can use raw pressure sensor data 1314 to calculate sleep metrics specifically tied to pressure sensor data. For example, user presence, movements, weight change, heartrate, and breathing rate can all be determined from raw pressure sensor data 1314. Additionally, an index or indexes stored by the sleep data cloud service 410b can identify users that are associated with pressure sensors, raw pressure sensor data, and/or pressure based sleep data.

The non-pressure sleep data 1316 can use other sources of data to calculate sleep metrics. For example, user-entered preferences, light sensor readings, and sound sensor readings can all be used to track sleep data. Additionally, an index or indexes stored by the sleep data cloud service 410b can identify users that are associated with other sensors and/or non-pressure sleep data 1316.

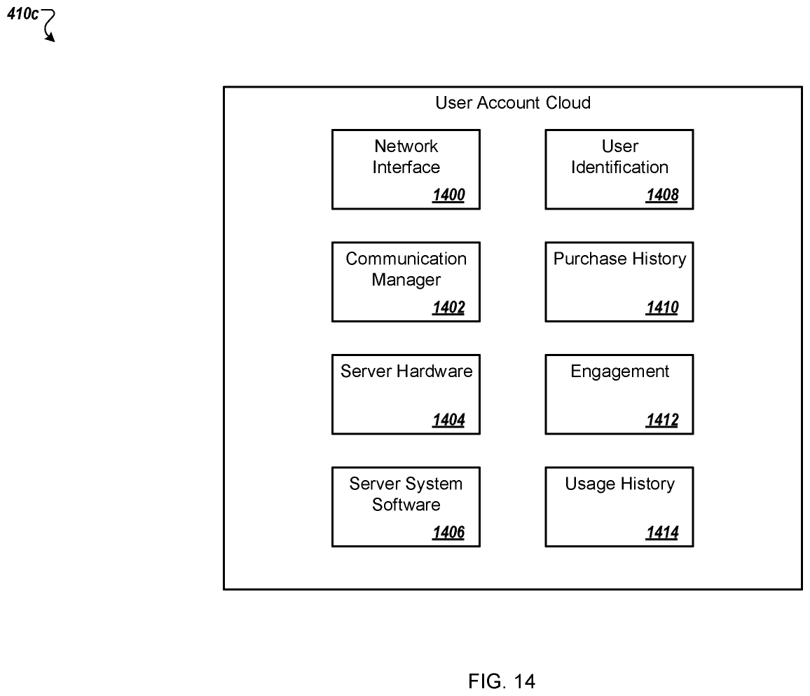

FIG. 14 is a block diagram of an example user account cloud service 410c that can be used in a data processing system associated with a bed system, including those described above with respect to FIGS. 1-3. In this example, the user account cloud service 410c is configured to record a list of users and to identify other data related to those users.

The user account cloud service 410c is shown with a network interface 1400, a communication manager 1402, server hardware 1404, and server system software 1406. In addition, the user account cloud service 410c is shown with a user identification module 1408, a purchase history module 1410, an engagement module 1412, and an application usage history module 1414.

The user identification module 1408 can include, or reference, data related to users of beds with associated data processing systems. For example, the users can include customers, owners, or other users registered with the user account cloud service 410c or another service. Each user can have, for example, a unique identifier, and user credentials, demographic information, or any other technologically appropriate information. Each user can also have user-inputted preferences pertaining to the user's bed system (e.g., firmness settings, heating/cooling settings, inclined and/or declined positions of different regions of the bed, etc.), ambient environment (e.g., lighting, temperature, etc.), and/or peripheral devices (e.g., turning on or off a television, coffee maker, security system, alarm clock, etc.).

The purchase history module 1410 can include, or reference, data related to purchases by users. For example, the purchase data can include a sale's contact information, billing information, and salesperson information that is associated with the user's purchase of the bed system. Additionally, an index or indexes stored by the user account cloud service 410c can identify users that are associated with a purchase of the bed system.

The engagement 1412 can track user interactions with the manufacturer, vendor, and/or manager of the bed and or cloud services. This engagement data can include communications (e.g., emails, service calls), data from sales (e.g., sales receipts, configuration logs), and social network interactions. The engagement data can also include servicing, maintenance, or replacements of components of the user's bed system.

The usage history module 1414 can contain data about user interactions with one or more applications and/or remote controls of a bed. For example, a monitoring and configuration application can be distributed to run on, for example, computing devices 412. The computing devices 412 can include a mobile phone, laptop, tablet, computer, smartphone, and/or wearable device of the user. The computing devices 412 can also include a central controller or hub device that can be used to control operations of the bed system and one or more peripheral devices. Moreover, the computing devices 412 can include a home automation device. The application that is presented to the user via the computing devices 412 can log and report user interactions for storage in the application usage history module 1414. Additionally, an index or indexes stored by the user account cloud service 410c can identify users that are associated with each log entry. User interactions that are stored in the application usage history module 1414 can optionally be used to determine or otherwise predict user preferences and/or settings for the user's bed and/or peripheral devices that can improve the user's overall sleep quality.

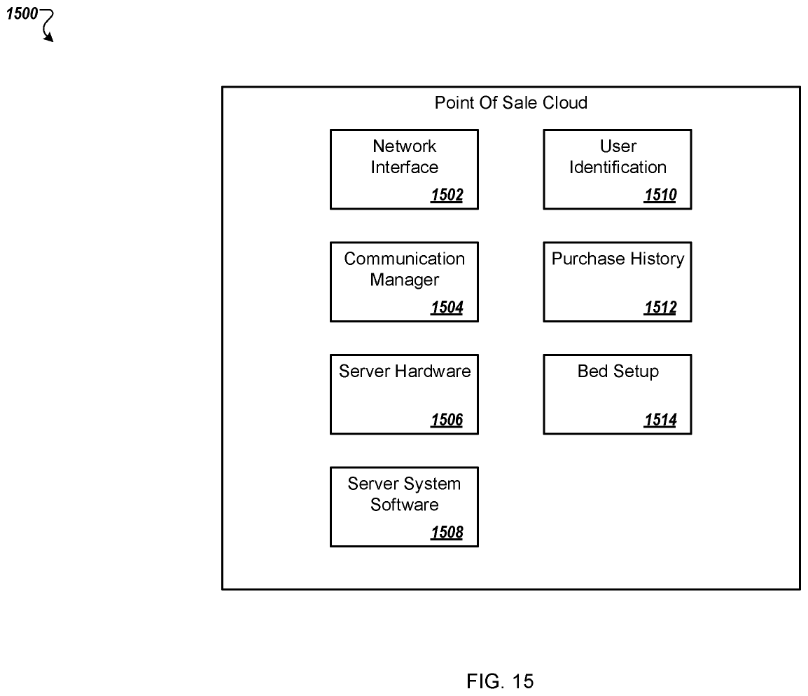

FIG. 15 is a block diagram of an example point of sale cloud service 1500 that can be used in a data processing system associated with a bed system, including those described above with respect to FIGS. 1-3. In this example, the point of sale cloud service 1500 is configured to record data related to users' purchases, specifically purchases of bed systems described herein.

The point of sale cloud service 1500 is shown with a network interface 1502, a communication manager 1504, server hardware 1506, and server system software 1508. In addition, the point of sale cloud service 1500 is shown with a user identification module 1510, a purchase history module 1512, and a bed setup module 1514.

The purchase history module 1512 can include, or reference, data related to purchases made by users identified in the user identification module 1510. The purchase information can include, for example, data of a sale, price, and location of sale, delivery address, and configuration options selected by the users at the time of sale. These configuration options can include selections made by the user about how they wish their newly purchased beds to be setup and can include, for example, expected sleep schedule, a listing of peripheral sensors and controllers that they have or will install, etc.

The bed setup module 1514 can include, or reference, data related to installations of beds that users purchase. The bed setup data can include, for example, a date and address to which a bed is delivered, a person who accepts delivery, configuration that is applied to the bed upon delivery (e.g., firmness settings), name or names of a user or users who will sleep on the bed, which side of the bed each user will use, etc.

Data recorded in the point of sale cloud service 1500 can be referenced by a user's bed system at later dates to control functionality of the bed system and/or to send control signals to peripheral components according to data recorded in the point of sale cloud service 1500. This can allow a salesperson to collect information from the user at the point of sale that later facilitates automation of the bed system. In some examples, some or all aspects of the bed system can be automated with little or no user-entered data required after the point of sale. In other examples, data recorded in the point of sale cloud service 1500 can be used in connection with a variety of additional data gathered from user-entered data.

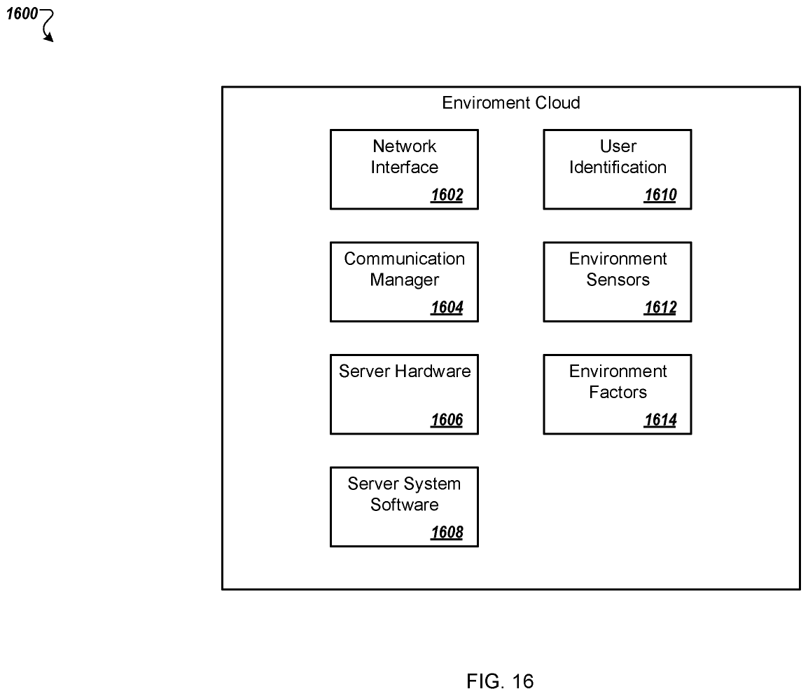

FIG. 16 is a block diagram of an example environment cloud service 1600 that can be used in a data processing system associated with a bed system, including those described above with respect to FIGS. 1-3. In this example, the environment cloud service 1600 is configured to record data related to users' home environment.

The environment cloud service 1600 is shown with a network interface 1602, a communication manager 1604, server hardware 1606, and server system software 1608. In addition, the environment cloud service 1600 is shown with a user identification module 1610, an environmental sensors module 1612, and an environmental factors module 1614.

The environmental sensors module 1612 can include a listing and identification of sensors that users identified in the user identification module 1610 have installed in and/or surrounding their bed. These sensors may include any sensors that can detect environmental variables, including but not limited to light sensors, noise/audio sensors, vibration sensors, thermostats, movement sensors (e.g., motion), etc. Additionally, the environmental sensors module 1612 can store historical readings or reports from those sensors. The environmental sensors module 1612 can then be accessed at a later time and used by one or more of the cloud services described herein to determine sleep quality and/or health information of the users.

The environmental factors module 1614 can include reports generated based on data in the environmental sensors module 1612. For example, the environmental factors module 1614 can generate and retain a report indicating frequency and duration of instances of increased lighting when the user is asleep based on light sensor data that is stored in the environment sensors module 1612.

In the examples discussed here, each cloud service 410 is shown with some of the same components. In various configurations, these same components can be partially or wholly shared between services, or they can be separate. In some configurations, each service can have separate copies of some or all of the components that are the same or different in some ways. Additionally, these components are only provided as illustrative examples. In other examples, each cloud service can have different number, types, and styles of components that are technically possible.

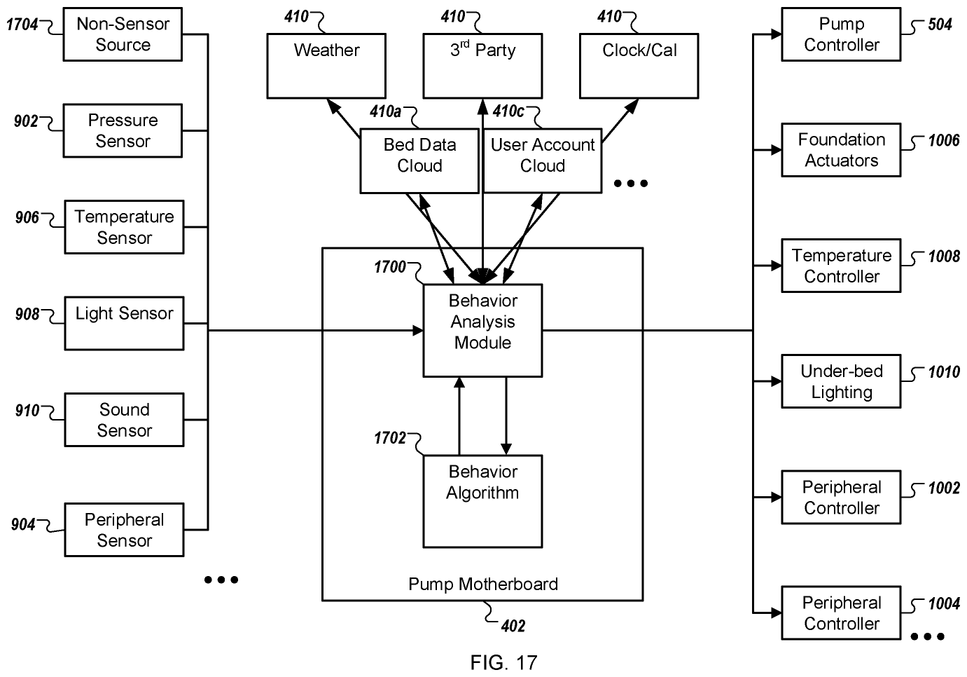

FIG. 17 is a block diagram of an example of using a data processing system associated with a bed (e.g., a bed of the bed systems described herein, such as in FIGS. 1-3) to automate peripherals around the bed. Shown here is a behavior analysis module 1700 that runs on the pump motherboard 402. For example, the behavior analysis module 1700 can be one or more software components stored on the computer memory 512 and executed by the processor 502.

In general, the behavior analysis module 1700 can collect data from a wide variety of sources (e.g., sensors 902, 904, 906, 908, and/or 910, non-sensor local sources 1704, cloud data services 410a and/or 410c) and use a behavioral algorithm 1702 (e.g., one or more machine learning models) to generate one or more actions to be taken (e.g., commands to send to peripheral controllers, data to send to cloud services, such as the bed data cloud 410a and/or the user account cloud 410c). This can be useful, for example, in tracking user behavior and automating devices in communication with the user's bed.

The behavior analysis module 1700 can collect data from any technologically appropriate source, for example, to gather data about features of a bed, the bed's environment, and/or the bed's users. Some such sources include any of the sensors of the sensor array 406 that is previously described (e.g., including but not limited to sensors such as 902, 904, 906, 908, and/or 910). For example, this data can provide the behavior analysis module 1700 with information about a current state of the environment around the bed. For example, the behavior analysis module 1700 can access readings from the pressure sensor 902 to determine the pressure of an air chamber in the bed. From this reading, and potentially other data, user presence in the bed can be determined. In another example, the behavior analysis module 1700 can access the light sensor 908 to detect the amount of light in the bed's environment. The behavior analysis module 1700 can also access the temperature sensor 906 to detect a temperature in the bed's environment and/or one or more microclimates in the bed. Using this data, the behavior analysis module 1700 can determine whether temperature adjustments should be made to the bed's environment and/or components of the bed in order to improve the user's sleep quality and overall comfortability.

Similarly, the behavior analysis module 1700 can access data from cloud services and use such data to make more accurate determinations of user sleep quality, health information, and/or control of the user's bed and/or peripheral devices. For example, the behavior analysis module 1700 can access the bed cloud service 410a to access historical sensor data 1212 and/or advanced sleep data 1214. Other cloud services 410, including those previously described can be accessed by the behavior analysis module 1700. For example, the behavior analysis module 1700 can access a weather reporting service, a 3^{rd} party data provider (e.g., traffic and news data, emergency broadcast data, user travel data), and/or a clock and calendar service. Using data that is retrieved from the cloud services 410, the behavior analysis module 1700 can more accurately determine user sleep quality, health information, and/or control of the user's bed and/or peripheral devices.

Similarly, the behavior analysis module 1700 can access data from non-sensor sources 1704. For example, the behavior analysis module 1700 can access a local clock and calendar service (e.g., a component of the motherboard 402 or of the processor 502). The behavior analysis module 1700 can use the local clock and/or calendar information to determine, for example, times of day that the user is in the bed, asleep, waking up, and/or going to bed.

The behavior analysis module 1700 can aggregate and prepare this data for use with one or more behavioral algorithms 1702. As mentioned, the behavioral algorithm 1702 can include machine learning models. The behavioral algorithms 1702 can be used to learn a user's behavior and/or to perform some action based on the state of the accessed data and/or the predicted user behavior. For example, the behavior algorithm 1702 can use available data (e.g., pressure sensor, non-sensor data, clock and calendar data) to create a model of when a user goes to bed every night. Later, the same or a different behavioral algorithm 1702 can be used to determine if an increase in air chamber pressure is likely to indicate a user going to bed and, if so, send some data to a third-party cloud service 410 and/or engage a peripheral controller 1002 or 1004, foundation actuators 1006, a temperature controller 1008, and/or an under-bed lighting controller 1010.

In the example shown, the behavioral analysis module 1700 and the behavioral algorithm 1702 are shown as components of the pump motherboard 402. However, other configurations are possible. For example, the same or a similar behavioral analysis module 1700 and/or behavioral algorithm 1702 can be run in one or more cloud services, and resulting output can be sent to the pump motherboard 402, a controller in the controller array 408, or to any other technologically appropriate recipient described throughout this document.

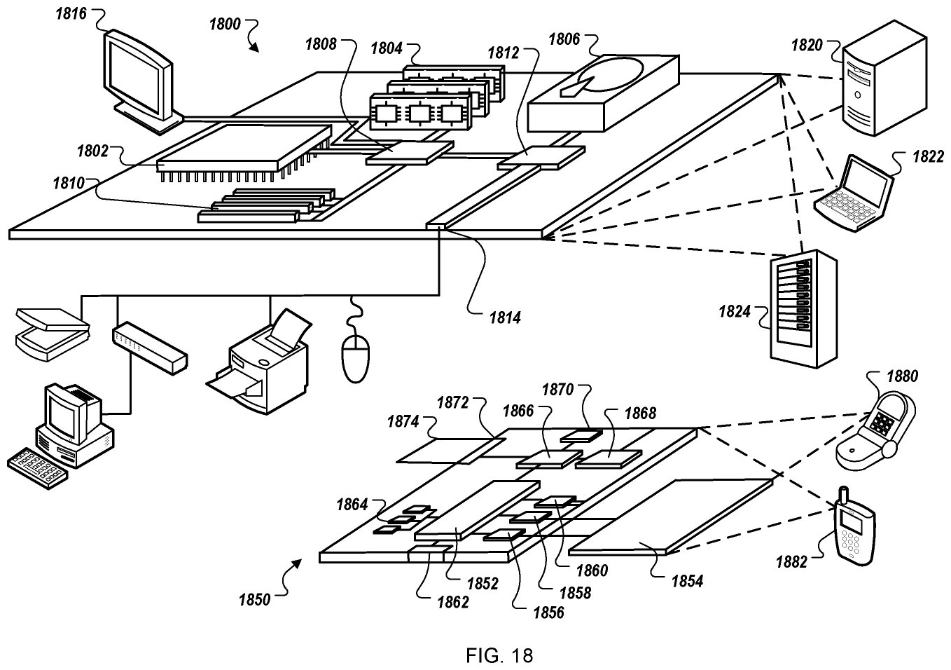

FIG. 18 shows an example of a computing device 1800 and an example of a mobile computing device that can be used to implement the techniques described here. The computing device 1800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1800 includes a processor 1802, a memory 1804, a storage device 1806, a high-speed interface 1808 connecting to the memory 1804 and multiple high-speed expansion ports 1810, and a low-speed interface 1812 connecting to a low-speed expansion port 1814 and the storage device 1806. Each of the processor 1802, the memory 1804, the storage device 1806, the high-speed interface 1808, the high-speed expansion ports 1810, and the low-speed interface 1812, are interconnected using various busses, and can be mounted on a common motherboard or in other manners as appropriate. The processor 1802 can process instructions for execution within the computing device 1800, including instructions stored in the memory 1804 or on the storage device 1806 to display graphical information for a GUI on an external input/output device, such as a display 1816 coupled to the high-speed interface 1808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1804 stores information within the computing device 1800. In some implementations, the memory 1804 is a volatile memory unit or units. In some implementations, the memory 1804 is a non-volatile memory unit or units. The memory 1804 can also be another form of computer-readable medium, such as a magnetic or optical disk.

The storage device 1806 is capable of providing mass storage for the computing device 1800. In some implementations, the storage device 1806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1804, the storage device 1806, or memory on the processor 1802.

The high-speed interface 1808 manages bandwidth-intensive operations for the computing device 1800, while the low-speed interface 1812 manages lower bandwidth-intensive operations. Such allocation of functions is exemplary only. In some implementations, the high-speed interface 1808 is coupled to the memory 1804, the display 1816 (e.g., through a graphics processor or accelerator), and to the high-speed expansion ports 1810, which can accept various expansion cards (not shown). In the implementation, the low-speed interface 1812 is coupled to the storage device 1806 and the low-speed expansion port 1814. The low-speed expansion port 1814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1800 can be implemented in a number of different forms, as shown in the figure. For example, it can be implemented as a standard server 1820, or multiple times in a group of such servers. In addition, it can be implemented in a personal computer such as a laptop computer 1822. It can also be implemented as part of a rack server system 1824. Alternatively, components from the computing device 1800 can be combined with other components in a mobile device (not shown), such as a mobile computing device 1850. Each of such devices can contain one or more of the computing device 1800 and the mobile computing device 1850, and an entire system can be made up of multiple computing devices communicating with each other.

The mobile computing device 1850 includes a processor 1852, a memory 1864, an input/output device such as a display 1854, a communication interface 1866, and a transceiver 1868, among other components. The mobile computing device 1850 can also be provided with a storage device, such as a micro-drive or other device, to provide additional storage. Each of the processor 1852, the memory 1864, the display 1854, the communication interface 1866, and the transceiver 1868, are interconnected using various buses, and several of the components can be mounted on a common motherboard or in other manners as appropriate.

The processor 1852 can execute instructions within the mobile computing device 1850, including instructions stored in the memory 1864. The processor 1852 can be implemented as a chipset of chips that include separate and multiple analog and digital processors. The processor 1852 can provide, for example, for coordination of the other components of the mobile computing device 1850, such as control of user interfaces, applications run by the mobile computing device 1850, and wireless communication by the mobile computing device 1850.

The processor 1852 can communicate with a user through a control interface 1858 and a display interface 1856 coupled to the display 1854. The display 1854 can be, for example, a TFT (Thin-Film-Transistor Liquid Crystal Display) display or an OLED (Organic Light Emitting Diode) display, or other appropriate display technology. The display interface 1856 can comprise appropriate circuitry for driving the display 1854 to present graphical and other information to a user. The control interface 1858 can receive commands from a user and convert them for submission to the processor 1852. In addition, an external interface 1862 can provide communication with the processor 1852, so as to enable near area communication of the mobile computing device 1850 with other devices. The external interface 1862 can provide, for example, for wired communication in some implementations, or for wireless communication in other implementations, and multiple interfaces can also be used.

The memory 1864 stores information within the mobile computing device 1850. The memory 1864 can be implemented as one or more of a computer-readable medium or media, a volatile memory unit or units, or a non-volatile memory unit or units. An expansion memory 1874 can also be provided and connected to the mobile computing device 1850 through an expansion interface 1872, which can include, for example, a SIMM (Single In Line Memory Module) card interface. The expansion memory 1874 can provide extra storage space for the mobile computing device 1850, or can also store applications or other information for the mobile computing device 1850. Specifically, the expansion memory 1874 can include instructions to carry out or supplement the processes described above, and can include secure information also. Thus, for example, the expansion memory 1874 can be provide as a security module for the mobile computing device 1850, and can be programmed with instructions that permit secure use of the mobile computing device 1850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1864, the expansion memory 1874, or memory on the processor 1852. In some implementations, the computer program product can be received in a propagated signal, for example, over the transceiver 1868 or the external interface 1862.

The mobile computing device 1850 can communicate wirelessly through the communication interface 1866, which can include digital signal processing circuitry where necessary. The communication interface 1866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1868 using a radio-frequency. In addition, short-range communication can occur, such as using a Bluetooth, WiFi, or other such transceiver (not shown). In addition, a GPS (Global Positioning System) receiver module 1870 can provide additional navigation- and location-related wireless data to the mobile computing device 1850, which can be used as appropriate by applications running on the mobile computing device 1850.

The mobile computing device 1850 can also communicate audibly using an audio codec 1860, which can receive spoken information from a user and convert it to usable digital information. The audio codec 1860 can likewise generate audible sound for a user, such as through a speaker, e.g., in a handset of the mobile computing device 1850. Such sound can include sound from voice telephone calls, can include recorded sound (e.g., voice messages, music files, etc.) and can also include sound generated by applications operating on the mobile computing device 1850.

The mobile computing device 1850 can be implemented in a number of different forms, as shown in the figure. For example, it can be implemented as a cellular telephone 1880. It can also be implemented as part of a smart-phone 1882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

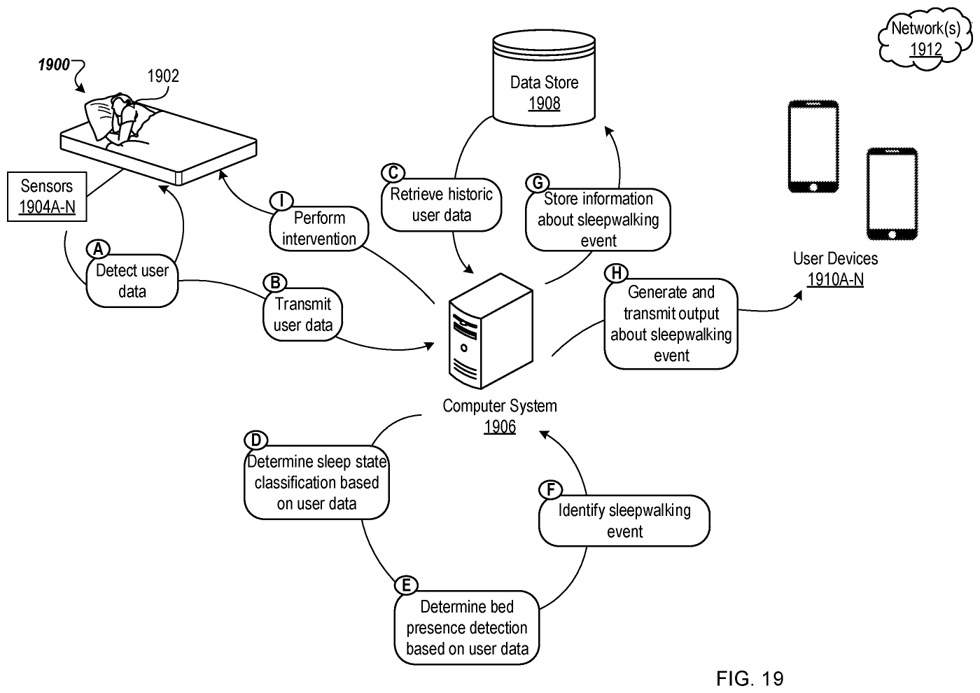

FIG. 19 is a conceptual diagram for detecting and preventing sleepwalking events of a user 1902 in a bed system 1900. Components of the bed system 1900, such as sensors 1904A-N, can be in communication with a computer system 1906 via network(s) 1912 (e.g., wired and/or wireless communication). The computer system 1906 can also communicate, via the network(s) 1912, with a data store 1908 and user devices 1910A-N.

In brief, the bed system 1900 can be a smart bed or another sleep system as described throughout this disclosure. The sensors 1904A-N can be pressure sensors. In some implementations, the sensors 1904A-N can be load cells. In some implementations, the sensors 1904A-N can include sensors on wearable devices and/or other sensors worn by the user 1902 (e.g., smart watch, heartrate monitor, smart clothes, smart bracelet, sometimes called a smart band, etc.). The computer system 1906 can be configured to detect and prevent sleepwalking events (e.g., somnambulism) of the user 1902. The computer system 1906 can be remote from the bed system 1900 and/or can be part of the bed system 1900 (e.g., a controller of the bed system 1900). The computer system 1906 can also be any other computing device, network of devices, network of computing systems, and/or cloud-based system, as described throughout this disclosure. The data store 1908 can be any type of storage device and/or cloud-based storage system that can be used for storing historic data about the user 1902 and other users of other bed and/or sleep systems. The user devices 1910A-N can be any type of computing device, including but not limited to mobile phones, smartphones, wearable devices, laptop, computer, tablet, etc., that may be used by the user 1902 or other relevant users. The other relevant users can include family members of the user 1902, healthcare providers associated with the user 1902, etc.

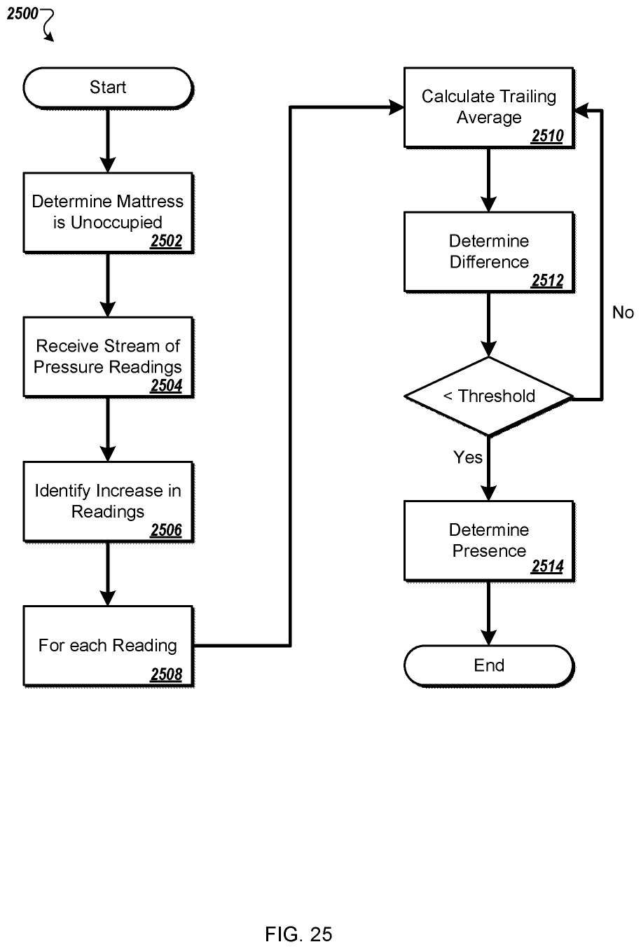

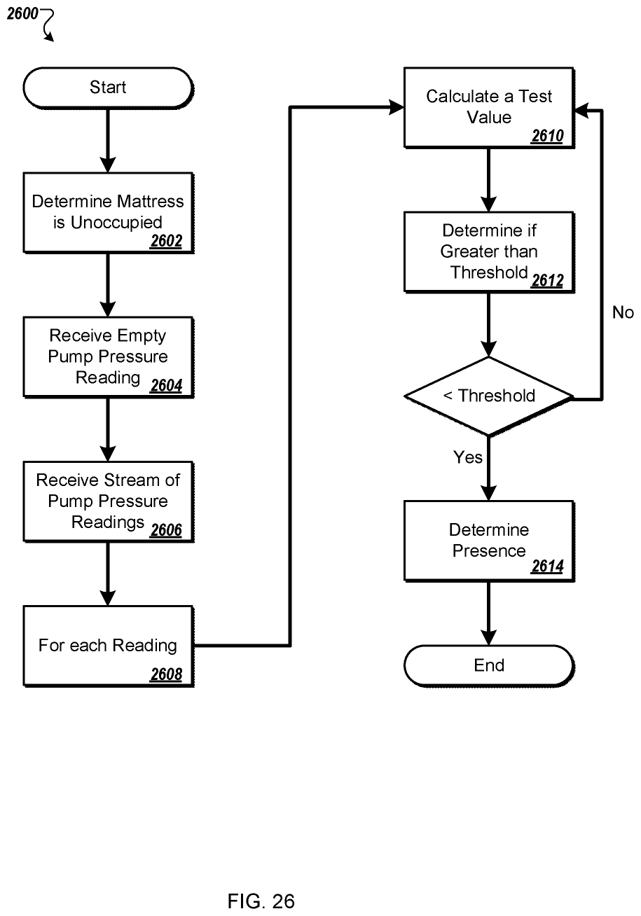

The sensors 1904A-N can detect user data in block A. The user data can be continuously detected by the sensors 1904A-N. The user data can also be determined at one or more predetermined time intervals. The user data can include, but is not limited to, BCG signals and other biometrics that can be used with the sleep state classification techniques described herein. The sensors 1904A-N can also detect data such as pressure data and/or temperature data in the bed system 1900. This data can also be used with the sleep state classification and/or bed exit detection techniques described herein. For example, pressure changes in an air chamber or chambers of the bed system 1900 and/or changes in temperature in a microclimate of the bed system 1900 can be used to determine if the user 1902 is in the bed system 1900 and when the user 1902 exits the bed system 1900, as described further below in reference to FIGS. 25-26.

In block B, the sensors 1904A-N can transmit the user data to the computer system 1906. The data can be transmitted to the computer system 1906 in real-time and/or near real-time, as the data is collected/detected by the sensors 1904A-N. Therefore, the data can be continuously transmitted to the computer system 1906. In some implementations, the data can be transmitted in batches, at one or more predetermined time intervals (e.g., every 5 minutes, every 10 minutes, at or after each sleep state, once during a sleep session, twice during a sleep session, etc.).

The computer system 1906 can also receive historic user data in block C. The historic user data can be about the user from prior sleep sessions. For example, the historic user data can include prior detections of sleepwalking events for the user 1902. The prior detections can be used to predict sleepwalking onset during a current sleep session and determine one or more interventions to prevent the predicted onset. The prior detections can also be used to more accurately detect a sleepwalking event. In some implementations, the historic user data can include prior detections of sleepwalking events for other users (e.g., users from a same or similar geographic location/region, users with one or more same or similar demographics as the user 1902, users with a similar timing of the sleepwalking event etc.), which can then be used by the computer system 1906 to train sleep state classifiers and/or more accurately detect sleepwalking and preventing the onset of somnambulism in the user 1902. Block C can be performed before, during, or after one or more other blocks described herein. For example, block C can be performed before block B. Block C can also be performed before block A.

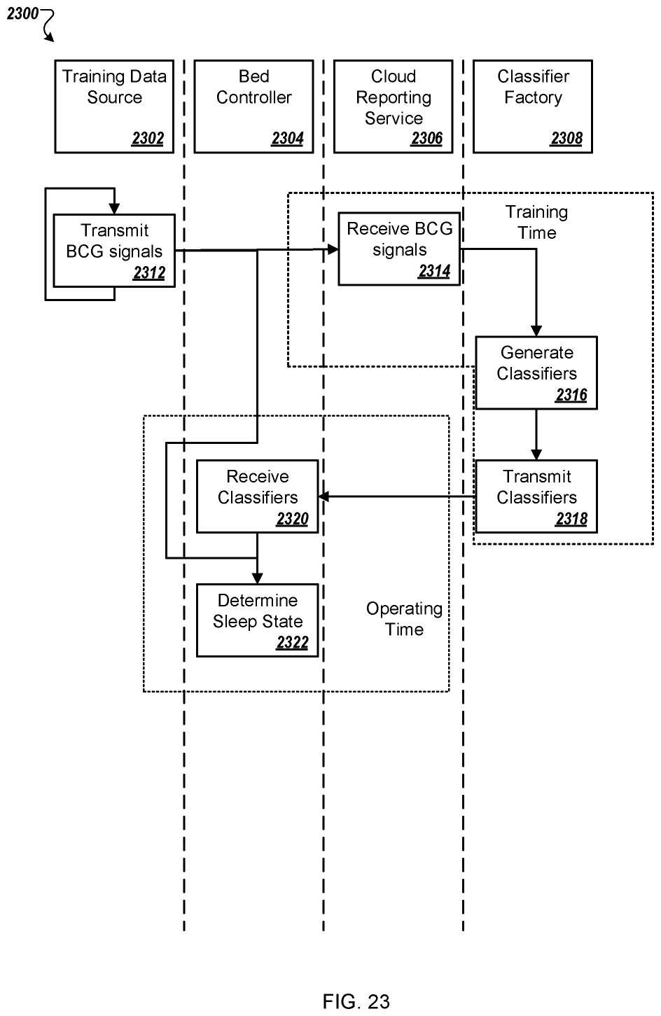

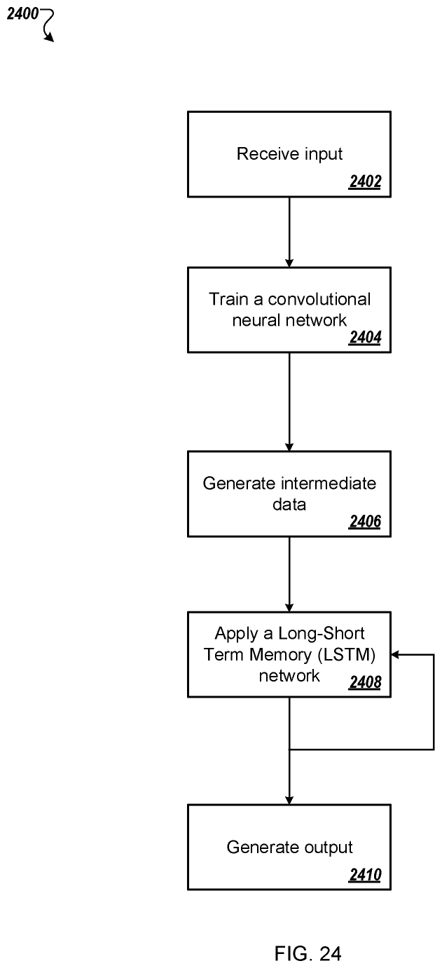

The computer system 1906 can determine sleep state classification based on the user data (block D). Refer to FIGS. 23-24 for additional discussion about determining sleep state classification.

The computer system 1906 can also determine bed presence detection based on the user data (block E). Refer to FIGS. 25-26 for additional discussion about determining bed presence detection. In some implementations, block E can be performed at the same time as block D and/or before block D.

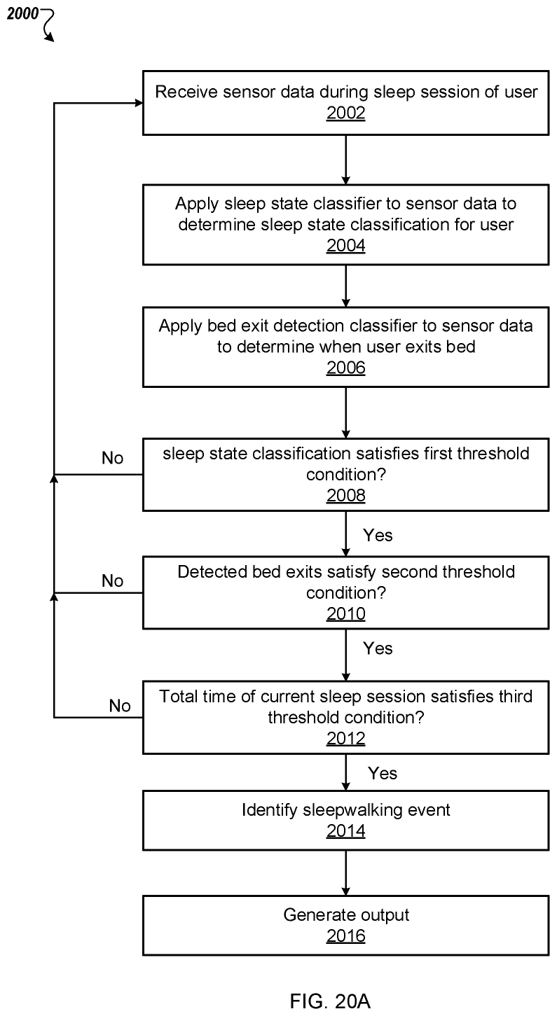

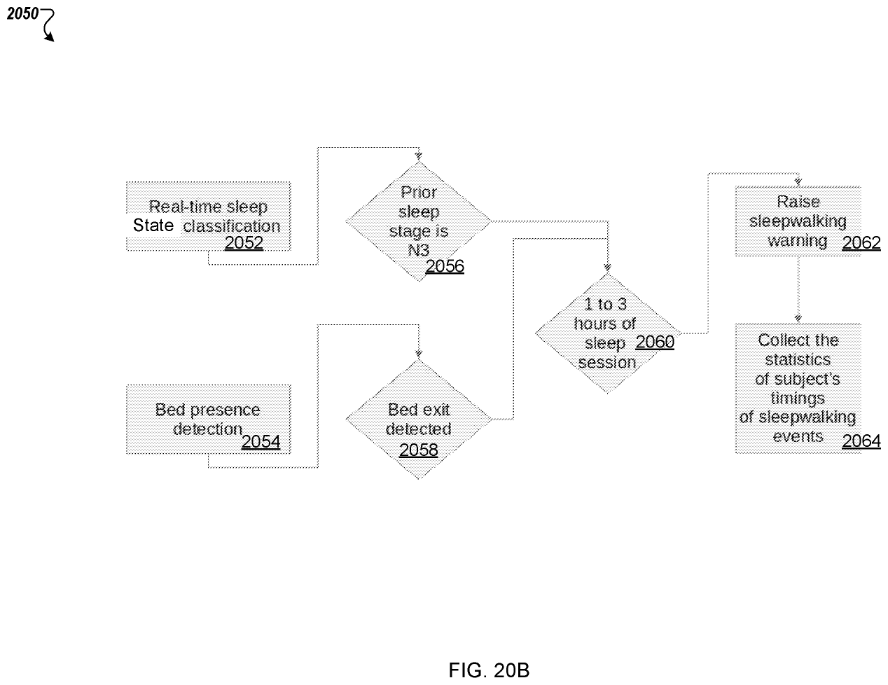

The computer system 1906 can identify a sleepwalking event in block F. In block F, the computer system 1906 can also predict a probability of sleepwalking onset for the user 1902 during the current sleep session. The sleepwalking event can be identified based on determinations in blocks D and E. For example, as described further in FIGS. 20A-B, the computer system 1906 can determine whether the sleep state classification (block D) is N3, whether the user is detected as exiting the bed (block E), and that a current time is within 1 to 3 hours since sleep onset (e.g., 1 to 2 sleep cycles). If each of these conditions is satisfied, the computer system 1906 can identify the sleepwalking event in block F.

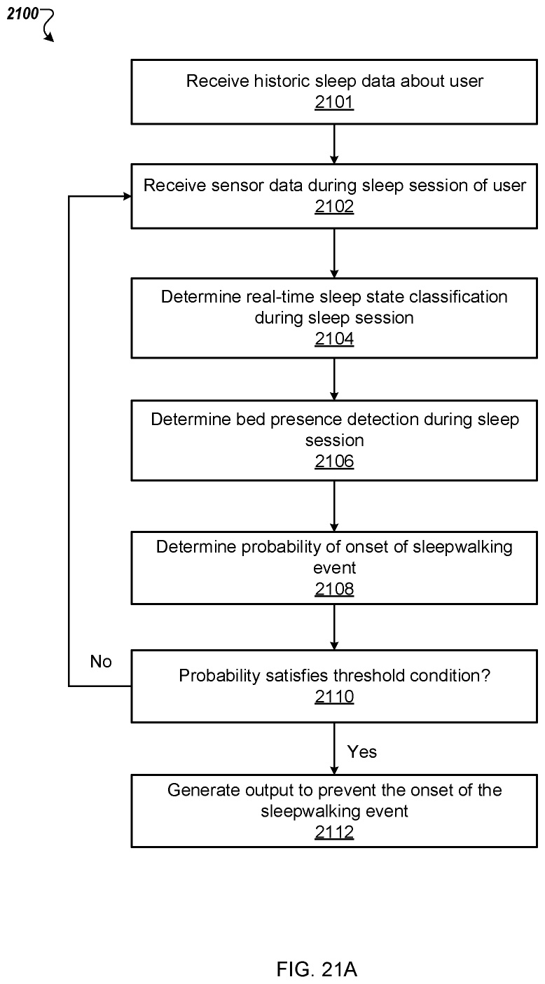

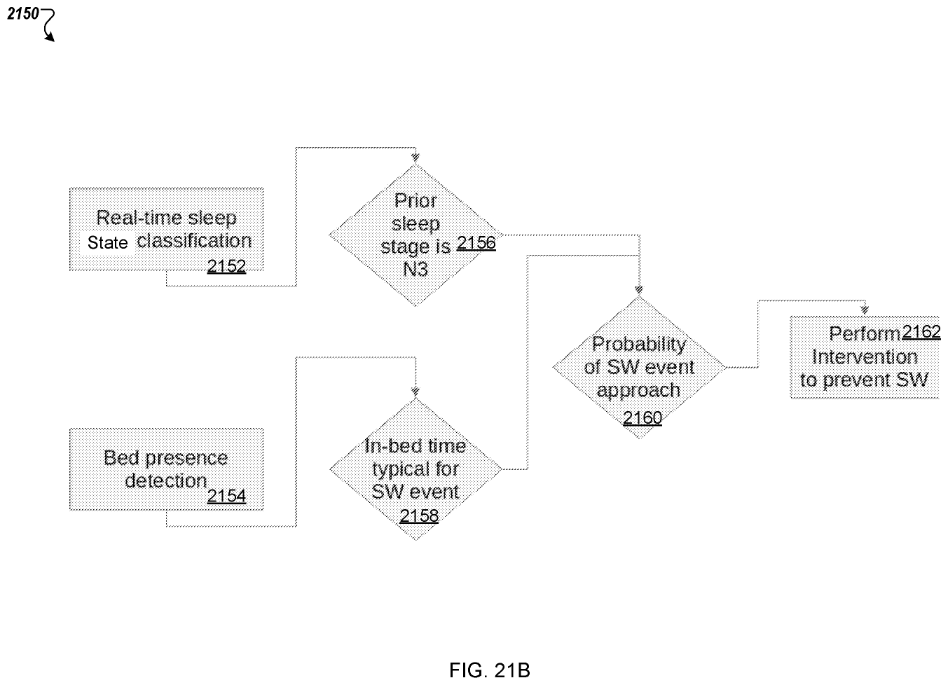

Moreover, the computer system 1906 can predict the probability of sleepwalking onset as described further in FIGS. 21A-B. For example, the computer system 1906 can determine whether the sleep state classification (block D) is N3 and whether a bed presence time for the user 1902 (block E) corresponds to the historic user data (block C) of prior sleepwalking events. If these conditions are satisfied, the computer system 1906 can predict the probability that the user 1902 is likely to sleepwalk within some threshold amount of time from the current time.

The computer system 1906 can store information about the sleepwalking event in the data store 1908 in block G. For example, the computer system 1906 can store information about the identified sleepwalking event, such as a time at which the user was detected as exiting the bed system 1900. The information can then be retrieved at a later time as part of the historic user data that is used to determine the probability of sleepwalking onset during a current sleep session and prevent the sleepwalking onset.

In block H, the computer system 1906 can generate and transmit output about the sleepwalking event to one or more of the user devices 1910A-N. For example, when the computer system 1906 identifies the sleepwalking event in block F, the computer system 1906 can generate a notification for presentation to the user 1902 and/or a relevant healthcare provider of the user 1902. The notification can indicate that the user 1902 sleepwalked during their sleep session. The notification can include at least some of the information about the identified sleepwalking event that is stored in the data store 1908 (block G). In some implementations, the output in block H can be part of an intervention performed by the computer system 1902 in real-time, to prevent sleepwalking onset during a current sleep session.

In block I, the computer system 1906 can perform an intervention based on the sleepwalking event. For example, if the computer system 1906 determines the probability of sleepwalking onset to satisfy a threshold condition (e.g., the probability exceeds a threshold value or range), then the computer system 1906 can generate and transmit instructions to the bed system 1900 that can cause a controller of the bed system 1900 to adjust the bed system 1900 in such a way that prevents the onset of somnambulism. For example, as described further in reference to FIGS. 21A-B, the intervention can include increasing a temperature of a microclimate of the bed system 1900 such that the user 1902's CBT can decrease, thereby advancing N3 sleep and preventing the onset of sleepwalking. As another example, the intervention can include delivering sensory stimulation (e.g., audio) to the user 1902 to shallow the user's sleep and make N3 sleep occur later, thereby preventing the onset of sleepwalking.

In some implementations, one or more firmness adjustments can be made to the bed system 1900 as an intervention in block I. Adjusting the firmness of a mattress, for example, can increase detectability of out-of-bed events for users having insomnia. Moreover, firmness can be adjusted to increase sensitivity of detecting out-of-bed events associated with sleepwalking. In some implementations, firmness adjustments can be made to indirectly modify sleep architecture. After all, by increasing comfort of the user, their deep sleep can last longer, thereby reducing a possibility that the user will experience a sleepwalking event.

As described throughout this disclosure, the computer system 1906 may not perform block I. For example, the computer system 1906 can perform the techniques described herein in order to detect sleepwalking events and collect information about sleepwalking patterns/routines of the user 1902. This information can then be stored in the data store (block G) and used during subsequent sleep sessions to predict whether the user 1902 is likely to sleepwalk and whether to perform an intervention. In some implementations, the computer system 1906 may perform block I but may not perform blocks G and/or H. For example, the computer system 1906 can receive real-time user data during a current sleep session and compare it to the historic user data to determine the probability of sleepwalking onset during the current sleep session. The computer system 1906 can then perform an intervention in block I without performing blocks G and H in order to prevent onset of sleepwalking during the current sleep session. One or more other combinations of the blocks A-I that are described in FIG. 19 may be performed.

FIG. 20A is a flowchart of a process 2000 for detecting a sleepwalking event of a user. The process 2000 can be performed during and/or throughout a sleep session of the user. In some implementations, the process 2000 can be continuously performed during the sleep session of the user, even if a sleepwalking event is not detected during a beginning of the sleep session (e.g., during a first 1 to 2 sleep cycles). After all, it is possible that the user may sleepwalk during later sleep cycles during the sleep session. The sleepwalking event can be detected based on a combination of sleep state classification and bed exit detection. Sleep state classifiers can be trained, using machine learning techniques, such as a machine learning model, to be more sensitive to deep sleep, thereby ensuring greater accuracy in detecting sleep states in which sleepwalking (e.g., somnambulism) is likely to occur, such as N3 (refer to FIG. 23 for sleep state classifier training). The process 2000 can be performed in order to learn timing and nature of sleepwalking events of the user, which can then be used in real-time to determine interventions that can prevent onset of sleepwalking events. Refer to FIGS. 21A-B for additional discussion about preventing the onset of sleepwalking events. Therefore, the process 2000 can be performed to determine that the user is sleepwalking based on detection of the user exiting the bed during a particular sleep state and the detection of the user exiting the bed falling within a threshold duration since the beginning of the user's sleep session.

The process 2000 can be performed by the computer system 102 and/or any of the components of the data processing system 400 described herein. In some implementations, the computer system 102 can be the data processing system 400. One or more other systems, computing systems, devices, network of devices, and/or cloud-based systems can be used to perform the same or a similar process. For illustrative purposes, the process 2000 is described from the perspective of a computer system.

Referring to the process 2000 in FIG. 20A, the computer system can receive sensor data during a sleep session of a user in block 2002, as described throughout this disclosure. As described herein, the data can be received from at least one sensor. The at least one sensor can be part of a bed system. The at least one sensor can also be another type of sensor that is in communication with the computer system, including but not limited to a wearable device.

In block 2004, the computer system can apply a sleep state classifier to the sensor data to determine a sleep state classification for the user. Machine learning techniques can be used to determine the sleep state classification for the user. For example, the computer system can provide, as input to the sleep state classifier, a first portion of the sensor data. The sleep state classifier was trained using machine learning techniques such as a machine learning model to determine the user's sleep states during the sleep session. The computer system can receive, as output from the sleep state classifier, a sleep state classification for the user. Refer to FIGS. 23-24 for additional discussion about determining the sleep state classification for the user.

The computer system can also apply a bed exit detection classifier to the sensor data to determine when the user exits the bed in block 2006. Machine learning techniques can be used to determine and detect bed exits of the user. For example, the computer system can provide, as input to the bed exit detection classifier, a second portion of the sensor data. The bed exit detection classifier was trained using machine learning techniques to determine when the user exits the bed throughout the sleep session. The computer system can receive, as output from the bed exit detection classifier, a bed exit detection classification for the user during the current sleep session. Refer to FIGS. 25-26 for additional discussion about determining bed exits of the user.

Referring to both blocks 2004 and 2006, machine learning techniques such as logistic regression models and training and deep neural networks can be used to accurately determine the sleep state classification for the user and detect bed exits of the user.

In block 2008, the computer system can determine whether the sleep state classification satisfies a first threshold condition. The sleep state, as described throughout this disclosure, can be a continuum that characterizes sleep depth. The first threshold condition can therefore be applied to the sleep state. For example, when the sleep state value is high, this can be correlated to a sleep stage of N3 (e.g., deep sleep). The first condition can be whether the sleep state classification is a deep sleep state, such as N3. As described herein, a user is more likely to sleepwalk during deeper sleep states, such as N3. Therefore, if the user is not in N3, then the user is not likely to sleepwalk and the process 2000 may not continue, in some implementations.

If the first threshold condition is satisfied (meaning the sleep state classification is a deep sleep state), the computer system can determine whether the detected bed exit(s) of the user satisfies a second threshold condition in block 2010. The second threshold condition can be whether the user has been detected as exiting the bed (e.g., detection of at least one bed exit event). If the user is in a deep sleep state and has exited the bed (e.g., within some threshold amount of time since onset of the deep sleep state), then the computer system can determine that the user is likely sleepwalking. On the other hand, if the user is in the deep sleep state but has not exited the bed, then the computer system can determine that the user is not sleepwalking and thus the process 2000 may not continue, in some implementations.

If the second threshold condition is satisfied, the computer system can determine whether a total time of the current sleep session satisfies a third threshold condition in block 2012. The computer system can therefore determine a duration of the sleep session of the user based on the sensor data received in block 2002. The third threshold condition can be whether the total time from a start of the current sleep session is within a threshold range. The threshold range can be approximately 1 to 3 hours (or a time period of 3 hours or less). The threshold range can be approximately 1 to 2 sleep cycles, in some implementations. Sometimes, the third threshold condition may not be excluding, meaning that sleepwalking events can still be detected if the duration of the sleep session exceeds the threshold range. For example, a particular user may experience sleepwalking events at the end of a sleep session, for example 4 to 6 hours after sleep onset. Although these events occur outside the range of the third threshold condition, they may still be detected for this particular user. A combination of sleep state classification and the total time of the current sleep session can be used to accurately determine what stage of sleep the user is experiencing and whether a sleepwalking event is in fact detected. Therefore, the first, second, and third threshold conditions may be satisfied for the computer system to accurately detect a sleepwalking event of the user.

If the third threshold condition is satisfied, the computer system can identify a sleepwalking event of the user in block 2014. After all, if the user is in a deep stage of sleep, they have been detected as exiting the bed, and the user is within 1 to 3 hours of falling asleep, then it is likely the user is sleepwalking. Therefore, the computer system can determine that the user is sleepwalking as a function of determining the sleep state classification, the bed presence of the user (e.g., detected bed exits), and a duration of the sleep session of the user.

The computer system can then generate output about the sleepwalking event in block 2016. The computer system can generate output to be presented at at least one output device (e.g., user device) and based on a determination that the user is sleepwalking. Generating the output can include generating a notification, warning, or other type of message indicating that a sleepwalking event was detected/identified. The notification can include information about the sleepwalking event, such as at what time the sleepwalking event was detected and/or when during the user's sleep session (or sleep cycle) the sleepwalking event was detected. The notification can be transmitted to a user device associated with the user. For example, the notification can be presented in a mobile application at the user device when the user wakes up from their current sleep session (e.g., the next morning). The notification can also be transmitted to a user device of a healthcare provider associated with the user. The notification can be, in some implementations, machine instructions to control an automation device. For example, the notification can include instructions that cause a medicine dispenser to stop dispensing or delivering medication to the user. The notification can include instructions that cause one or more other devices to automatically perform actions intended to ensure safety of the user.

In some implementations, generating the output can include storing information about the identified sleepwalking event in a data store. This information can then be retrieved and used in real-time to determine whether the user is likely to sleepwalk during a subsequent/future sleep session. For example, the computer system can store the sleep state classification, the bed exit detection classification, and/or the total time of the current sleep session (e.g., total time between the sleep session starting and the sleepwalking event being detected) in the data store. The computer system can also store the identification of the sleepwalking event in the data store. The identification of the sleepwalking event can include, but is not limited to, information about a time during the user's sleep session when the sleepwalking event was identified.

Referring back to block 2008, if the first threshold condition is not satisfied, the computer system can return to block 2002 in which the computer system continues to receive sensor data during the sleep session of the user.

Referring back to block 2010, if the second threshold condition is not satisfied, the computer system can also return to block 2002.

Referring back to block 2012, if the third threshold condition is not satisfied, the computer system can also return to block 2002.

FIG. 20B is a flowchart of another process 2050 for detecting a sleepwalking event of a user. The process 2050 is similar to or the same as the process 2000 described in FIG. 20A. The process 2050 can be performed by the computer system 102 and/or any of the components of the data processing system 400 described herein. In some implementations, the computer system 102 can be the data processing system 400. One or more other systems, computing systems, devices, network of devices, and/or cloud-based systems can be used to perform the same or a similar process. For illustrative purposes, the process 2050 is described from the perspective of a computer system.

Referring to the process 2050 in FIG. 20B, the computer system can perform real-time sleep state classification in block 2052, as described throughout this disclosure. The computer system can also perform bed presence detection in block 2054, as described throughout this disclosure. Blocks 2052 and 2054 can be performed simultaneously. Block 2052 can be performed before block 2054. In some implementations, block 2054 may be performed before block 2052.

In block 2056, the computer system can determine whether a sleep state determined during the classification in block 2052 is N3. In other words, the computer system can determine it predicted a deep stage of sleep for the user, such as N3. If the sleep state is not N3, then the process 2050 can stop or the computer system can return to blocks 2052 and 2054 and continue to classify the user's sleep states and/or detect bed presence. If the sleep state is N3, then the computer system can proceed to block 2060, if the condition in block 2058 is also satisfied/true.

In block 2058, the computer system can determine whether at least one bed exit was detected. If the user is not detected as exiting the bed, then the process 2050 can stop or the computer system can return to blocks 2052 and 2054 and continue to classify the user's sleep states and/or detect bed presence. If at least one bed exit event is detected, then the computer system can proceed to block 2060, assuming the condition in block 2056 is also satisfied/true.

If both the sleep state is N3 and at least one bed exit was detected, the computer system can determine whether a current duration of the sleep session is 1 to 3 hours since sleep onset (block 2060). In other words, the computer system can determine whether the total time from start of the current sleep session is within a range of approximately 1 to 3 hours. The range can also be approximately 1 to 2 sleep cycles, in some implementations. If the current duration is not 3 hours or less, then the process 2050 can stop or the computer system can return to blocks 2052 and 2054.

If, on the other hand, the current duration of the sleep session is 3 hours or less since sleep onset, the computer system can raise a sleepwalking warning in block 2062. Raising the sleepwalking warning can include generating a notification, message, or alert indicating that a sleepwalking event was detected. The notification can include additional information about the sleepwalking event, such as timing of the sleepwalking event. The notification can be transmitted to a device of a healthcare provider associated with the user. The notification can also be transmitted to a device of the user. For example, the notification can be received and presented at the user's device in the morning, when the user wakes up from the current sleep session.

The computer system can also collect statistics (e.g., information) about the user's timing(s) of sleepwalking events during the sleep session (block 2064). Collecting the information about the detected sleepwalking events can include storing the information in a data store, as described herein. The information can then be used in real-time to predict the onset of sleepwalking events in subsequent sleep sessions and perform an intervention to prevent the onset of the predicted sleepwalking events.

The processes 2000 and 2050 of FIGS. 20A-B can be performed to detect sleepwalking events over one night, multiple nights, or a predetermined amount of nights (e.g., sleep sessions). In some implementations, for example, the processes 2000 and 2050 can be continuously performed, each night that the user goes to sleep in their bed (e.g., during each sleep session). The processes 2000 and 2050 to detect sleepwalking events can also be performed to determine somnambulism onset prevention for a next night, as described in further detail in FIGS. 21A-B. The techniques described in FIGS. 21A-B may, for example, be performed after the computer system has learned the sleepwalking habits of the user, as described in the processes 2000 and 2005. As a result of performing the techniques described in FIGS. 20-21, the computer system can perform actions (e.g., interventions) during subsequent sleep sessions to prevent the onset of sleepwalking in a gentle way to ensure the user continues to experience uninterrupted and quality sleep.

FIG. 21A is a flowchart of a process 2100 for preventing onset of a sleepwalking event of a user. The process 2100 can be performed to preemptively trigger an intervention that can prevent onset of a sleepwalking event, where that sleepwalking event may occur around the same time during each sleep session. In other words, the process 2100 can be performed to prevent the user from sleepwalking upon detection of user activity in a bed (e.g., bed presence detection, a particular sleep state, such as N3, a combination of bed presence detection and a particular sleep state, etc.) that corresponding to historic sleep data for the user. The techniques described in the process 2100 can be performed to prevent the user from sleepwalking within a threshold amount of time of detecting the user activity in the bed. The threshold amount of time, as described further below, can be approximately 0 to 15 minutes. The process 2100 can be performed to make deep sleep deeper or increase differentiations between deep sleep and a wake sleep state to ensure that the user does not sleepwalk in order to prevent the sleepwalking event.

The detection techniques described in reference to FIGS. 20A-B can be used to accurately determine whether the user is likely to sleepwalk during a current sleep session and, if so, perform an intervention to prevent onset of that sleepwalking event. In other words, the techniques described in FIGS. 21A-B can be performed as a subset of the techniques described in FIGS. 20A-B. For example, the techniques described in FIGS. 20A-B can be performed to detect sleepwalking events of the user, then the techniques described in FIGS. 21A-B can be performed to preemptively prevent the onset of sleepwalking events during the user's current sleep session.

The process 2100 can be performed by the computer system 102 and/or any of the components of the data processing system 400 described herein. In some implementations, the computer system 102 can be the data processing system 400. One or more other systems, computing systems, devices, network of devices, and/or cloud-based systems can be used to perform the same or a similar process. For illustrative purposes, the process 2100 is described from the perspective of a computer system.

Referring to the process 2100 in FIG. 21A, the computer system can receive historic sleep data about the user in block 2101. The computer system can retrieve the historic sleep data from a data store, as described herein. The historic sleep data can include timing information for prior detected sleepwalking events of the user during prior sleep sessions. The historic sleep data can also include any of the information that is determined and/or stored as part of the detection processes 2000 and 2050 described in FIGS. 20A-B.

The computer system can also receive sensor data during a sleep session of the user in block 2102, as described throughout this disclosure.

Block 2101 and 2102 can be performed in any order. Blocks 2101 and 2102 can also be performed simultaneously. In some implementations, block 2101 may be performed when the computer system is ready to perform block 2106 and/or block 2108.

In block 2104, the computer system can determine real-time sleep state classification during the sleep session. Block 2104 can be performed as part of block 2004 in the process 2000 of FIG. 20A. In some implementations, the computer system can determine the real-time sleep state classification in block 2104 based on sensing and analyzing how much the user sweats during the sleep session. For example, users typically sweat during deeper stages of sleep. By monitoring amounts that the user sweats during the current sleep session (e.g., using temperature and/or humidity sensors of the bed, for example), the computer system can identify when the user is entering or is in deeper stages of sleep, such as N3.

In block 2106, the computer system can determine bed presence detection during the sleep session. Block 2106 can be performed as part of block 2006 in the process 2000 of FIG. 20A. As part of block 2106, the computer system can determine whether bed exit detection(s) during the current sleep session align with, or are within a threshold range, of timing for bed exit detections in the historic sleep data.

The computer system may determine a probability of onset of sleepwalking during the current sleep session in block 2108. The probability of the onset of the sleepwalking can indicate a likelihood that the user will experience the sleepwalking event within a threshold amount of time from a current time during the sleep session. This determination can be made based on the determinations of blocks 2104 and 2106. This determination can also be made based at least in part on the historic sleep data about the user. For example, the computer system can generate the probability of the onset of sleepwalking for the user based on a determination that the sleep state classification satisfies a first threshold condition and that the bed exit detection classification satisfies a second threshold condition. The first threshold condition can be an N3 sleep state.

The second threshold condition can be a typical time at which the user has been detected in bed when sleepwalking events occurred. Therefore, the bed exit detection classification can indicate a time at which bed presence of the user is detected. The bed exit detection classification for the user may satisfy the second threshold condition based on a time at which the bed presence of the user is detected corresponding to historic data of detected sleepwalking events for the user. The bed exit detection classification can correspond to the historic data (e.g., information about prior detected sleepwalking events of the user during prior sleep sessions) based on the bed presence detection classification indicating an in-bed time of the user that is similar to (or within a threshold range of) in-bed times associated with prior detected sleepwalking events of the user.

For example, if the user is detected as being in bed at a time (or within a range of time) that does not correspond to prior times that the user was detected to be sleepwalking, then the second threshold condition may not be satisfied (e.g., the user is in N3 sleep and is detected to be in bed at 2:15 AM but historically at or around this time the user has not been detected as sleepwalking). The second threshold condition may be satisfied if the user is detected as being in bed at the time or within the range of time that corresponds to the user's prior sleepwalking events (e.g., the user is in N3 sleep and is detected to be in bed at 3:20 AM and historically the user had sleepwalked around 3:35-3:40 AM).

The probability can be determined using an estimation of a probability function. The probability can be a numeric value. The value can be on a scale of values, such as 0 to 100, where 0 is a lowest probability and 100 is a highest probability. One or more other scales can also be used to determine the probability. Moreover, the probability can be determined for a threshold amount of time from the current time, as described above. The threshold amount of time can be within 15 minutes from the current time. One or more other threshold amounts of time can be used, such as a next 5 minutes, 10 minutes, 20 minutes, 25 minutes, 30 minutes, etc.

As an illustrative example, the historic sleep data can indicate that the user typically sleepwalks around 1:20 AM. A current time during the user's sleep session can be 4:20 AM. The computer system can determine the likelihood that the user will sleepwalk around 4:20 AM to be lower than if the current time was 1:10 AM or 1:30 AM, for example. After all, it is less likely that the user will sleepwalk around 4:20 AM if, historically, the user has not or has rarely sleepwalked around that time. Therefore, the computer system can determine a low probability of sleepwalking onset for the current time in the user's sleep session.

As another illustrative example, the historic sleep data can indicate that the user typically sleepwalks three times a night and that as the night goes on, the user is less likely to sleepwalk again. During the current sleep session, the computer system can generate a high probability of sleepwalking onset early in the sleep session for a first sleepwalking event if the historic sleep data indicates prior instances of sleepwalking onset early in the user's sleep session (e.g., the current time in the sleep session is within a threshold range of the time(s) at which the user had previously sleepwalked). The computer system may generate a lower probability of sleepwalking onset later in the current sleep session for a third sleepwalking event, for example, if the user has already been detected as sleepwalking earlier in the sleep session and/or the user was more likely to sleepwalk once or multiple times earlier in the sleep session. Therefore, as the sleep session goes on through the night, the probability that the user will sleepwalk may decrease.

In block 2110, the computer system can determine whether the probability satisfies a threshold condition. The threshold condition can be a numeric value. The threshold condition can vary depending on the historic sleep data associated with the user. Therefore, the threshold condition can be different for each user, and can be based on the learning/detection phase described in the processes 2000 and 2050 in FIGS. 20A-B. In some implementations, the threshold condition can be a numeric value indicating likelihood that the user will sleepwalk within a threshold amount of time of the computer system determining that the user is in N3 sleep and the user is in bed around a time that prior sleepwalking events were detected. The threshold amount of time can be approximately 15 minutes from the current time (e.g., the time of the computer system's determination).

For example, a user who is more likely to sleepwalk during a sleep session can have a lower threshold condition than a user who is less likely to sleepwalk during a sleep session. The lower threshold condition can make the computer system more sensitive to identify when the user is likely to sleepwalk during the sleep session. This can be beneficial when the user frequently sleepwalks. A higher threshold condition can be beneficial for a user who infrequently sleepwalks because the higher threshold condition can ensure that the computer system is not over-inclusive by identifying normal activity during the user's sleep session (e.g., waking up to go to the bathroom or get water, for example) as potential sleepwalking events. The user who infrequently sleepwalks may frequently get up during the night to get a glass of water, for example. Thus, the higher threshold condition can ensure that the computer system does not identify each time the user gets up during the night as sleepwalking events.

The threshold condition can be adjusted in terms of sensitivity and specified for a particular user. As an example, a user who has a history of prior sleep walking, can be associated with a threshold that ensures at least 95% sensitivity even if specificity is 70%. As another example, another user who does not have a prior history of sleep walking, can be associated with a specificity that is greater than 95%. The different threshold conditions per user can be determined using a Receiving-Operator Curve (ROC). The ROC is a graphical plot that illustrates a connection/trade-off between sensitivity and specificity for possible cut-offs in sleepwalking event detection.

Moreover, in some implementations, firmness of the bed can be adjusted to improve detectability of the user exiting the bed. Detecting in and out of bed events can be more precise/accurate with lower firmness settings of the bed. Therefore, firmness of the bed can be adjusted automatically to increase sensitivity of detecting out of bed events associated with sleepwalking. If, historically, a particular user has a risk of sleep walking, then the computer system can automatically reduce a firmness level (e.g., pressure) of the user's bed by a predetermined amount to increase sensitivity of bed presence detection. The computer system can also selectively reduce the firmness of the user's bed based on sleep stage classification/determination in block 2104. For example, if the computer system determines that the user is entering a sleep stage in which they are most likely to sleep walk, the computer system can generate instructions that cause a pump of the bed to let out air from a mattress of the bed by a predetermined amount, thereby lowering the firmness level of the bed to increase sensitivity of bed presence detection during the current and/or next sleep stage(s) of the user.

In some implementations, the computer system may adjust the firmness level of the bed only if a current firmness level of the bed exceeds a threshold firmness level. If the current firmness level of the bed is less than the threshold firmness level, then the computer system may not generate instructions that cause the firmness of the bed to be adjusted to a lower firmness level/setting. After all, if the current firmness level is below the threshold firmness level, then the sensors of the bed already have higher sensitivity to detect out of bed events and user presence in the bed.

The firmness level can be assigned a numeric value on a scale of 1 to 100. One or more other scales may also be used (e.g., 1 to 50, 1 to 5, 1 to 10, 0 to 10, 0 to 100, etc.). A lower value on the scale can indicate that the bed is less firm and a higher value on the scale can indicate that the bed is more firm. As an illustrative example, the threshold firmness level can be 90, higher on the firmness scale. If the bed has a current firmness level between 90-100 (where 100 represents a bed having maximum firmness), the computer system can generate instructions to reduce the current firmness level by the threshold amount. The threshold amount can be between 1-3 firmness levels, where each firmness level can correspond to a different amount of pressure (e.g., measured in PSI) to be released from the bed (or within air chambers in an air mattress of the bed). The threshold amount can also be one or more other firmness levels, including but not limited to 5 firmness levels, 6 firmness levels, 7 firmness levels, 8 firmness levels, 9 firmness levels, 10 firmness levels, etc.

Referring back to block 2110, if the threshold condition is not satisfied, the computer system can return to block 2102 and continue to monitor the user's sleep session as described in the process 2100. As an illustrative example, the computer system may determine that the user is in a deep sleep state but that they are detected as being in bed (they have not exited the bed). The historic user data can indicate that around this same time, the user typically would sleepwalk (the user would exit the bed). The computer system can determine a low probability of sleepwalking onset at/around the current time in the user's sleep session. This probability may not satisfy the threshold condition because the user is not detected as exiting the bed, even though they are in the deep sleep state. Since the threshold condition is not satisfied, the computer system may not perform an intervention. After all, the user is unlikely or less likely to sleepwalk, so the computer system can continue to monitor the user's sleep session (e.g., by returning to block 2102) instead of performing an intervention.

If the threshold condition is satisfied, the computer system can proceed to block 2112. As an illustrative example, the computer system may determine that the user is in a deep sleep state and the historic user data can indicate that around this same time, the user typically would sleepwalk (the user would exit the bed). The computer system can determine a high probability of sleepwalking onset at/around the current time in the user's sleep session. This probability may satisfy the threshold condition. Since the threshold condition is satisfied, the computer system may perform an intervention. After all, the user is more likely to sleepwalk, so the computer system can perform an intervention to prevent the sleepwalking from occurring in the first place.

In block 2112, the computer system can generate output to prevent the onset of the sleepwalking event. The computer system can generate and perform the output within a threshold amount of time of determining that the probability satisfies the threshold condition. The threshold amount of time can be approximately 15 minutes or less from the time of the determination (e.g., the current time). One or more other threshold amounts of time can be used. For example, the threshold amount of time can vary per user based on the user's historic record of timing of sleepwalking events.

The output can be a form of intervention. The intervention can be intended to prevent sleepwalking from occurring. The computer system can generate one or more different types of output.

The output, for example, can be an intervention to adjust a microclimate of the user's bed. The computer system can generate instructions that are transmitted to a controller of the user's bed. The instructions can cause the controller, when executed, to activate a heating element in the bed to increase a temperature of the microclimate by a threshold amount (or within a threshold range). For example, the temperature of the microclimate can be increased until the temperature is within a range of approximately 25-36° C. In some implementations, the instructions can cause the controller to actuate the heating element to increase the temperature of the microclimate by 0.5 to 1° C. One or more other ranges of temperature increase can also be used to increase the microclimate as described herein.

Increasing the temperature of the microclimate of the bed can cause the user's core body temperature (CBT) to more quickly decrease. The quicker decrease in CBT can make deep sleep occur sooner, thereby preventing the sleepwalking from occurring. The instructions transmitted to the controller can also instruct warming in certain regions/areas of the bed. For example, the instructions can cause a foot warming element to warm a foot end of the bed by a threshold amount and/or until the temperature at the foot end of the bed is within a threshold temperature range. One or more other climate changes can be made to the bed in order to make deep sleep occur sooner and thus prevent the onset of sleepwalking.

The output can also include sensory stimulation that may cause the user to wake up, or transition to another sleep state other than deep sleep, within a range of time (e.g., 15 minutes) before the possible sleepwalking event occurs. As a result of waking up the user, the user can fall back into a normal state of sleep rather than deep sleep, thereby preventing the onset of the possible sleepwalking event. Delivering sensory stimulation can also delay the onset of deep sleep. The sensory stimulation can include audio and/or vibrations outputted by the bed or one or more devices, such as the user's device (e.g., smartphone, mobile phone, wearable device, laptop, tablet, etc.). Thus, when the user is detected as entering or approaching deeper sleep, the user's sleep can be shallowed by outputting audio such as the user's name. Hearing the user's name can cause the user to transition into a normal state of sleep rather than deep sleep. In other words, the user may wake up. The user's name can be outputted once. In some implementations, the user's name can be repeated until the computer system detects that the user is entering a normal state of sleep rather than a deep sleep state.

In some implementations in which the computer system monitors how and when the user sweats during the sleep session, the intervention can include adjusting temperature and/or ventilation of the bed to manipulate the user's sweating. For example, a temperature of the bed can be increased to decrease the user's CBT such that the user does not sweat and therefore does not enter deep sleep at the current time. As another example, the intervention can include activating a ventilation unit (e.g., fan) of the bed to dry the user's sweat and/or decrease the user's CBT. In such implementations, the computer system can determine and perform an intervention based on analysis of the users sweat instead of analyzing and determining the user's real-time sleep state classification(s).

In some implementations, the intervention can include generating instructions to adjust a firmness level of the bed. Increased comfortability of the user can cause deep sleep to last longer or to occur earlier during a sleep session, thereby delaying or otherwise avoiding the onset of sleep walking. The computer system can generate instructions that adjust the firmness level of the bed to increase user comfortability when the user is detected to be entering or currently is in deep sleep. Various firmness levels can be conducive to improved comfortability. Such firmness levels can vary depending on the preferences and historic sleep information of a particular user. For example, some users may experience increased comfortability if their bed is more firm while other users may experience increased comfortability if their bed is less firm. The computer system can leverage personal preferences and historic sleep information to generate appropriate instructions that adjust the firmness level of the particular user's bed, thereby preventing the onset of sleepwalking. Once the bed is adjusted to the new firmness level, the bed can remain at that firmness level until the user is detected as waking up, until a new sleep stage is detected (such as NREM), a predetermined amount of time has passed, or one or more other factors are met. Once the factors are met, the computer system can generate instructions that cause the firmness level of the bed to be adjusted to a firmness level before the first adjustment was made. The computer system can also generate instructions that cause the firmness level of the bed to be adjusted to a user-preferred firmness level.

In some implementations, another intervention can include generating instructions that cause an adjustable foundation/base of the bed to be adjusted. The adjustable foundation can be adjusted to improve safety of the user, should the user sleepwalk. For example, the computer system can generate instructions to cause the adjustable foundation to lower by a predetermined amount towards the ground. As a result, if the user does sleepwalk, the user may have a lower risk of getting injured by falling out of the bed. Therefore, the computer system can generate and perform the intervention based on generating and transmitting instructions to a controller of the bed that, when executed by the controller, cause the controller to lower the adjustable foundation of the bed to a threshold height. The threshold height can be the predetermined amount towards the ground. The threshold height can be a number value. For example, the threshold height can be within a range of approximately 20 to 23 inches from the ground to a top of the mattress. This range can be beneficial to ensure safety of the user should they sleep walk and get out of bed. The threshold height can also be a human-relatable description (e.g., low enough that many users, or users of a particular demographic, can sit on the bed and place their heels on the floor, low enough to be comfortable, etc.). In some implementations, the threshold height can also be a user-specifiable height. Moreover, the threshold height can vary depending on age of the user. As users get older in age, they tend to get shorter. Therefore, the threshold height can be lower (e.g., closer to the ground) for users who are older than users who are young. The threshold height can vary depending on a threshold age. For example, the threshold height can be adjusted to a lower value (e.g., numeric, such as 20 inches from the ground to the top of the mattress) once the user's age is 40 years old or older. The threshold height can continue to be adjusted to a lower value as the user's age increases.

A default intervention can be adjusting the microclimate of the bed. In some implementations, the computer system can determine which intervention may be most effective to prevent somnambulism onset for the particular user. Then the computer system can perform the most effective intervention in block 2112. In some implementations, the computer system may perform one type of intervention for a first predicted sleepwalking event during the user's sleep session and then may perform another type of intervention for a second predicted sleepwalking event during the same sleep session. One or more other variations are also possible.

The computer system can then continue to perform the process 2100. After all, the computer system can continue to monitor the current sleep session to determine whether the user is likely to sleepwalk again. In some cases, once the user sleepwalks a first time during a sleep session, the user is less likely to sleepwalk again during that same sleep session. Therefore, in some implementations (e.g., when the historic sleep data indicates that the user typically sleepwalks once a night at a particular time), the process 2100 may stop if an intervention has been performed to prevent the user from sleepwalking a first time during the sleep session. In some implementations, the computer system can continue to monitor the user's sleep to detect sleepwalking events, as described in the processes 2000 and 2050 of FIGS. 20A-B.

FIG. 21B is a flowchart of another process 2150 for preventing onset of a sleepwalking event of a user. The process 2150 is similar to or the same as the process 2100 described in FIG. 21A. The process 2150 can be performed by the computer system 102 and/or any of the components of the data processing system 400 described herein. In some implementations, the computer system 102 can be the data processing system 400. One or more other systems, computing systems, devices, network of devices, and/or cloud-based systems can be used to perform the same or a similar process. For illustrative purposes, the process 2150 is described from the perspective of a computer system.

Referring to the process 2150 in FIG. 21B, the computer system can perform real-time sleep state classification in block 2152, as described throughout this disclosure. In block 2154, the computer system can perform bed presence detection, as described throughout this disclosure. Blocks 2152 and 2154 can be performed in any order. Blocks 2152 and 2154 can also be performed simultaneously (e.g., in parallel). In some implementations, one or more of the blocks 2152 and 2154 can be continuously performed during/throughout a user's sleep session. Moreover, as described herein, one or more of the blocks 2152 and 2154 can be performed as part of the detection phase described in reference to FIGS. 20A-B (e.g., refer to blocks 2052 and 2054 in the process 2050 of FIG. 20B).

In block 2156, the computer system can determine whether the sleep state classification of block 2152 is N3. If the sleep state classification is not N3, the process 2150 can end or the computer system can return to blocks 2152 and/or 2154 and continue to monitor the user's sleep session. If the sleep state classification is N3, the computer system can proceed to block 2160, assuming the condition of block 2158 is also satisfied/true.

In block 2158, the computer system can determine whether an in-bed time for the user, as determined by the bed presence detection of blocks 2154, is typical for prior identified sleepwalking events for the user. This determination can be made using information about prior occurrences of sleepwalking events for the user, which can be retrieved from a data store. If the in-bed time is not typical for the user, the process 2150 can end or the computer system can return to blocks 2152 and/or 2154 and continue to monitor the user's sleep session. If the in-bed time is typical, the computer system can proceed to block 2160, assuming the condition of block 2156 is also satisfied/true. Blocks 2156 and 2158 can be performed in any order. Moreover, the blocks 2156 and 2158 can be performed simultaneously, in some implementations.

If the prior sleep state is N3 (block 2156) and the in-bed time is typical for prior sleepwalking events of the user (block 2158), the computer system can determine a probability that a sleepwalking event is approaching in block 2160 (e.g., likelihood of onset of a sleepwalking event). The probability, as described herein, can be based on likelihood that the sleepwalking event will occur within a threshold amount of time from a current time. The threshold amount of time can, for example, be 15 minutes from the current time.

If the probability satisfies a threshold condition in block 2160, the computer system can perform an intervention to prevent the predicted sleepwalking event from occurring in block 2162. The threshold condition can be different for each user and based on historic sleepwalking data associated with the user, as described further in the process 2100 in FIG. 21A. If the probability, for example, is greater than a threshold value, the computer system can determine that the sleepwalking event is more likely to occur and thus an intervention should be performed. On the other hand, if the probability is less than the threshold value, the computer system can determine that the sleepwalking event is unlikely to occur (or less likely to occur) during the current sleep session and thus an intervention may not be performed at the current time. The computer system can continue to monitor the user's sleep session by returning to blocks 2152 and 2154 and repeating the process 2150.

The intervention, as described herein, can cause the user to wake up for a short period of time to avoid or otherwise delay deep sleep. As a result, the sleepwalking event can be prevented. The computer system may take advantage of the history of timing and sleep state of the user's sleepwalking events to influence sleep architecture and advance or delay N3 sleep, thereby preventing onset of future sleepwalking events. Advancing N3 can be accomplished by mildly increasing temperature of a microclimate of the user's bed (e.g., by increasing the temperature by 0.5° C.) to promote a faster decrease of CBT.

Other options to modify latency to N3 sleep can include delaying N3 sleep onset. An intervention that delays N3 sleep onset can include delivery of sensory stimulation. Delivering sensory stimulation, such as audio and/or vibration, can shallow sleep so that N3 sleep occurs later. This intervention can be beneficially used in situations where the user may experience somnambulism that can cause harm to the user or others.

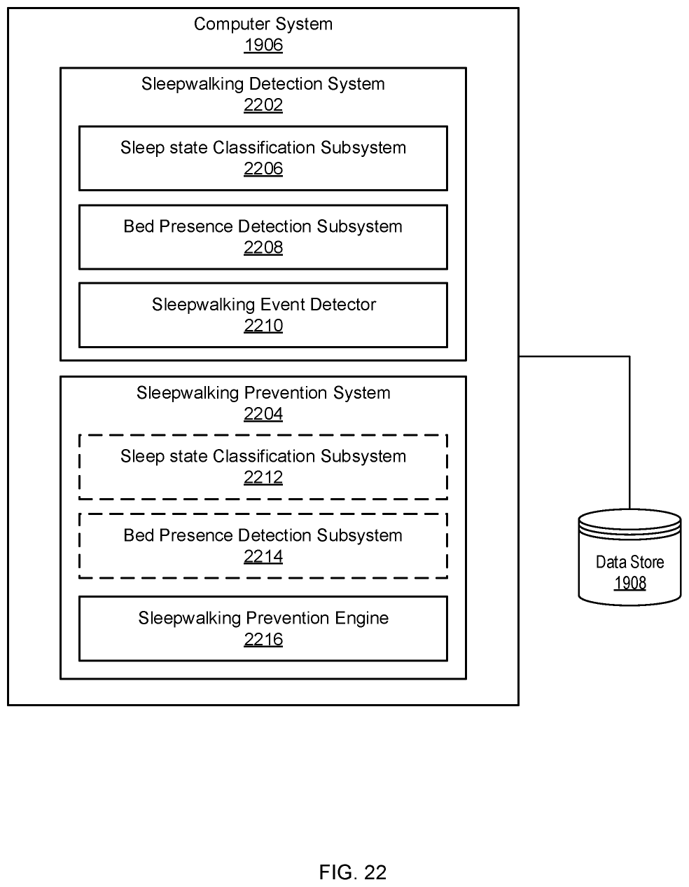

FIG. 22 is a system diagram of components of a computing system that can detect and prevent onset of a sleepwalking event of a user. The computing system can be the computer system 1906 described in reference to FIG. 19. In some implementations, the computing system can also be the data processing system 400 described herein.

The computer system 1906 can include a sleepwalking detection system 2202 and a sleepwalking prevention system 2204. In some implementations, the systems 2202 and 2204 can be combined into one subsystem of the computer system 1906. In some implementations, the systems 2202 and 2204 can be separate systems and may not be subsystems of the computer system 1906.

The sleepwalking detection system 2202 can include a sleep state classification subsystem 2206, a bed presence detection subsystem 2208, and a sleepwalking event detector 2210. The sleep state classification subsystem 2206 can be configured to perform techniques in identifying a current sleep state of a user based on sensed user data. Refer to FIGS. 23-24 for additional discussion about sleep state classification.

The bed presence detection subsystem 2208 can be configured to perform techniques in detecting when the user is present in the bed and when the user exits the bed during a sleep session. Refer to FIGS. 25-26 for additional discussion about bed presence detection.

The sleepwalking event detector 2210 can be configured to determine whether a sleepwalking event is detected. This determination can be made based on the techniques performed by the subsystems 2206 and 2208. The detector 2210 can also be configured to generate information about the detected sleepwalking event and store that information in the data store 1908, as described throughout this disclosure. Refer to the processes 2000 and 2050 in FIGS. 20A-B for additional discussion about detecting sleepwalking events of the user.

The sleepwalking prevention system 2204 can include a sleep state classification subsystem 2212, a bed presence detection subsystem 2214, and a sleepwalking prevention engine 2216. The subsystems 2212 and 2214 can be optionally part of the sleepwalking prevention system 2204. The subsystem 2212 can perform same or similar techniques as the sleep state classification subsystem 2206 of the sleepwalking detection system 2202. The subsystem 2214 can also perform same or similar techniques as the bed presence detection subsystem 2208 of the sleepwalking detection system 2202. In some implementations, the sleepwalking prevention system 2204 may not include the subsystems 2212 and 2214. Instead, the subsystems 2206 and 2208 of the sleepwalking detection system 2202 can perform the techniques described herein, which can then be used by the sleepwalking prevention engine 2216 to determine and perform an intervention.

Accordingly, the sleepwalking prevention engine 2216 can be configured to determine a probability that the user is likely to sleepwalk within a threshold amount of time from a current time that determinations are made by the subsystems 2212 and 2214 (or the subsystems 2206 and 2208, in some implementations). The engine 2216 can then determine whether the probability satisfies a threshold condition to generate and perform an intervention. The engine 2216 can retrieve the historic user sleep data from the data store 1908 and use that data to perform one or more of the techniques described herein. Refer to the processes 2100 and 2150 in FIGS. 21A-B for additional discussion about preventing the onset of sleepwalking during a current sleep session.

FIG. 23 is a swimlane diagram of an example process 2300 for training and using machine-learning classifiers to determine user sleep state. For clarity, the process 2300 is being described with reference to a particular set of components. However, other system or systems can be used to perform the same or a similar process.

In the process 2300, a bed system can use BCG signals from a training data source 2302 to categorize a user's sleep (or lack thereof). The bed system is able to use the BCG signals for a decision engine that classifies the user's sleep into one of a plurality of possible sleep states. Thus, the decision engine can use instantaneous heart rate (IHRs) time series, which are part of BCG signals, as an input to predict or otherwise determine sleep states of a user throughout the user's sleep session. The decision engine can determine sleep states that may include two stages (e.g., asleep or awake), five stages (e.g., awake, N1, N2, N3, and REM), three stages (e.g., awake, light sleep, deep sleep), or any other combination or set of stages.

In some implementations, the training data source 2302 can be an electrocardiography (ECG) source/device that collects signals from a user's body, such as body movement, heart rate, and breathing rate. However, other types of signals (e.g., BCG signals) such as those from which an IBI can be extracted may be used. This type of ECG source/device can include electrodes that are placed on the user's body. In some implementations, the training data source 2302 can be other wearable devices that track the user's body movement, heart rate, and/or breathing rate. Wearables can include smart watches, heart rate monitors, other straps, bracelets, rings, and/or mobile devices. In yet some implementations, the training data source 2302 can be sensors on a bed, such as pressure sensors. As described throughout this disclosure, the bed sensors can be configured to measure pressure changes on a top surface of the bed, which can indicate movement of the user (or lack thereof). One or more of the bed sensors can also be configured to measure changes in pressure in one or more air chambers, which can also indicate movement of the user. Moreover, one or more of the bed sensors can be configured to measure health conditions of the user, such as heart rate and breathing rate.

In some implementations, the training data source 2302 can be a repository, such as a database, or already collected BCG signals and other signals of a variety of users. These already collected signals can be annotated and labeled with different sleep states.

The training data source 2302 can collect sensor signals for a variety of different users. Those sensor signals can be annotated and tagged with different sleep states and used to train one or more machine learning models to detect different sleep states of users. Sleep state classifiers can then be transmitted to one or more beds for run-time use. The one or more beds that receive the classifiers can be different than the training data source 2302. In some implementations, one or more of the beds that receive the classifiers can be the same as the training data source 2302. In some implementations, during run-time use, the classifiers can be used by a computer system, such as the computer system 1906 described in FIG. 19, to determine sleep states of users of one or more different beds. The computer system can, in some implementations, be remote from the one or more different beds.

During run-time use, the sleep state classifiers can be used by the one or more beds to determine current sleep states of users during their sleep sessions. For example, during run-time use, a bed system can collect pressure signals indicating body movement, heart rate, and/or breathing rate of the user as the user rests on the bed. The bed system can apply the sleep state classifiers to the pressure signals in order to determine a current sleep state of the user.

In some implementations, sensor signals can be collected from a first bed, used to train one or more machine learning models to classify user sleep states, and then resulting classifiers can be transmitted back to the first bed and used during run-time. Thus, the process 2300 can be used to refine or otherwise improve one or more existing sleep state classifiers. As a result, the first bed can more accurately detect and determine different sleep states of the particular user(s) who uses the first bed.

In some implementations, sensor signals can be collected from a first bed, used to train one or more machine learning models to classify user sleep states, and then resulting classifiers can be transmitted to a second bed. The second bed can be different than the first bed. Thus, in this example, the process 2300 can be used to prepare the second bed to be able to determine user sleep states. In other words, the second bed might have just been manufactured and purchased by a user. Before the second bed is delivered to the user's home for installation and use, the second bed can be configured/calibrated to perform functions that it is intended to perform, such as detecting sleep states. Thus, the process 2300 can be performed to configure the second bed for detecting sleep states.

Referring to the process 2300, the training data source 2302 can transmit one or more BCG signals to a cloud computing service 2306 in 2312. The BCG signals can include polysomnographic sleep recordings and corresponding sleep states annotation. The BCG signals can include one or more heart beats (or IBI sequences), body movement, breathing rate, and/or heart rate signals, etc. Such signals can be measured from a variety of users and annotated with corresponding sleep states. In some implementations, as described above, the BCG signals can also include streams of pressure readings received from a bed system. The pressure readings can reflect pressure inside of an air bladder within the bed system. The pressure readings can also reflect health conditions of the user of the bed system, such as the user's body movement and/or heart rate.

The cloud reporting service 2306 can receive the BCG signals in 2314. For example, the training data source 2302 can transmit all signals or determine that some signals—and not others—should be transmitted to the cloud reporting service 2306 that is configured to receive signals and, in some cases, other types of data. The signals sent to the cloud reporting service 2306 may be unchanged by the training data source 2302, aggregated (e.g., averages, maximums and minimums, etc.), or otherwise changed by the training data source 2302. As described above, for example, the training data source 2302 can modify the signals by annotating them with sleep states. Another way that the training data source 2302 can modify the signals can be sending just heart rate signals instead of a combination of heart rate and breathing rate signals. Thus, the training data source 2302 can extract the heart rate signals out from the combination and transmit just the extract heart rate signals.

During training time, a classifier factory 2308 generates classifiers from the received BCG signals in 2316. The classifier factory 2308 can train classifiers by first obtaining a large set of pre-classified BCG signal variation patterns. For example, one bed or many beds may report pressure data to the cloud reporting service 2306. This pressure data may be tagged, recorded, and stored for analysis in the creation of pressure classifiers to be used by the bed controller 2304 and/or other bed controllers. This pressure data can indicate a variety of BCG signal patterns. The more data that is collected, the more likely a greater quantity of BCG signal patterns can be used for training. Accordingly, the more robust training datasets, the more likely the classifiers can accurately identify various types of BCG signal patterns that may exist during run-time use.

The classifier factory 2308 can generate features from the BCG signals. The stream of signals may be broken into buffers of, for example, 1 second, 2.125 seconds, 30 seconds, or 3 seconds, to generate features in time or frequency domains. Features can be extracted or otherwise identified in each of these buffers. As an illustrative example, features can include peaks and dips in detected heart rates and/or breathing rates. As another example, features can include detection of body movement (e.g., movement of the user's head, shoulders, arms, torso, legs, and/or feet).

In some cases, the classifier factory 2308 can generate features directly. In some cases (not shown), the bed controller 2304 and/or the training data source 2302 can generate features and send features (as opposed to pressure, as shown) to the cloud reporting service 2306.

For example, such features may include a maximum, minimum, or random pressure value. These features may be derived from the pressure readings within those buffers. For example, such features may include an average pressure, a standard deviation, a slope value that indicates an increase or decrease over time within that buffer, user motion, respiration measurement, cardiac measurement, and cardiorespiratory coupling measurement from the pressure variations. For example, rate/amplitude/duration of inhalation, rate/amplitude/duration of exhalation, rate of inhalation-exhalation cycle, the amplitude, width and location of fundamental frequency of breathing, the heart rate, rate of atrial depolarization, rate/amplitude/duration of atrial repolarization, rate/amplitude/duration of ventricular depolarization, rate of ventricular repolarization, etc. may be used. The values of the feature vectors may be in binary or numerical form. For each buffer, the values may be stored in a predetermined order creating a vector that is composed of a series of fields, where every vector has the same series of fields and data in those fields. Some other features may be computed from the transform domain representations of the pressure signal such as from the Fourier or Wavelet Transform coefficients.

As another example, the classifier factory can identify instances within the signals where the signals match a pattern or rules for a pattern. In one example, a constant or fluctuating pattern may be identified in the motion signal or at least one physiological signal (cardiac beats, breathing or cardiorespiratory coupling) derived from the BCG signals. Such patterns may be identified, and corresponding synthetic information about the pattern (e.g., timestamp, duration, rate of change, frequency of change, max change, slope of change, etc.) may be synthesized from the signal and/or other outside information (e.g., a real-time clock).

The classifier factory 2308 can also combine or reduce the features. For example, the extracted features can be combined using principal component analysis. For a principal component analysis of the features, the classifier factory 2308 can determine a subset of all features that are discriminant of the sleep state of the user. That is, the classifier factory 2308 can sort features into those features that are useful for determining state and those features that are less useful, and the more useful features may be kept. This process may be done on a trial-and-error basis, in which random combinations of features are tested. This process may be done with the use of one or more systematic processes. For example, a linear discriminant analysis or generalized discriminant analysis may be used.

In some cases, a proper subset of features may be selected out of the set of all available features. This selection may be done once per classifier if multiple classifiers are being created. Alternatively, this selection may be done once for a plurality or all classifiers if multiple classifiers are being created.

For example, a random (or pseudorandom) number may be generated and that number of features may be removed. In some cases, a plurality of features may be aggregated into a single aggregate feature. For example, for a case in which a plurality of fluctuating physiological patterns are identified in the BCG signals, the patterns and/or synthetic data related to the patterns may be aggregated. For example, the consecutive heart rate differences may be aggregated into a mean, a standard deviation, a minimum, and/or a maximum heart rate.

The classifier factory 2308 can also process the features. For example, the remaining features may then be processed to rationalize their values so that each feature is handled with a weight that corresponds to how discriminant the feature is. If a feature is found to be highly discriminant so that is highly useful in classifying state, that feature may be given a larger weight than other features. If a second feature is found to be less discriminant than other features, that second feature can be given a lower weight.

Once mapped into kernel space, the features can be standardized to center the data points at a predetermined mean and to scale the features to have unit standard deviation. This can allow the features to all have, for example, a mean value of 0 and a standard deviation of 1. The extracted features are then converted to a vector format using the same vector format as described above.

In some cases, the remaining features can be processed by applying a kernel function to map the input data into a kernel space. A kernel space allows a high-dimensional space (e.g., the vector space populated with vectors of feature data) to be clustered such that different clusters can represent different states. The kernel function may be of any appropriate format, including linear, quadratic, polynomial, radial basis, multilayer perceptron, or custom.

In some cases, the classifier factory 2308 can use machine-learning techniques that do not create features. For example deep learning networks such as convolutional networks, deep feed forward, or deep recurrent networks can be used. The classifier factory 2308 can train a dilated convolutional neural network (CNN) with the BCG signals in which the classifier factory 2308 can detect inter-beat intervals (IBIs) time series from the BCG signals (e.g., or ECG signals), remove missing data segments that are greater than a predetermined amount of time (e.g., 4 seconds), remove outliers that are out of range (e.g., by standard deviation or physiologic considerations), linearly interpolate and resample the IBIs (e.g., to 2 Hz), and normalize a local mean and standard deviation for each sleep session corresponding to each BCG signal (e.g., refer to FIGS. 20-21). Examples of physiologic considerations can include, but are not limited to removing IBIs not expected to occur in normal sleep (e.g., IBIs>2000 milliseconds corresponding to 30 beats per minute or IBI<500 milliseconds corresponding to 120 beats per minute or IBI<500 milliseconds (corresponding to 120 beats per minute.)

Thus, convolutional layers can be used by the classifier factor 2308 to learn local cardiac features. Dilated convolutional blocks can also be used to learn long-range features as sleep states related to temporal cardiac features can be contained in a long-time span.

The classifier factory 2308 can train the classifiers. For example, a pattern recognizer algorithm can use the vectors of extracted features and their corresponding sleep state labels as a dataset to train the classifiers with which new BCG signals can be classified. In some cases, this can include storing the classifiers with the training data for later use.

The classifier factory 2308 can transmit the classifiers in 2318 and the bed controller 2304 can receive the classifiers in 2320. For example, the classifier or classifiers created by the classifier factory 2308 can be transmitted to the bed controller 2304 and/or other bed controllers. As described herein, the classifiers can also be transmitted to a remote computer system, such as the computer system 1906. In some cases, the classifiers can be transmitted on non-transitory computer readable mediums like a compact disk (CD), a Universal Serial Bus (USB) drive, or other device. The classifiers may be loaded onto the bed controller 2304 and/or other bed controllers as part of a software installation, as part of a software update, or as part of another process. In some cases, the classifier factory 2308 can transmit a message to the bed controller 2304 and/or other bed controllers, and the message can contain data defining one or more classifiers that use a stream of pressure readings to classify the bed into one of a plurality of sleep states. In some configurations, the classifier factory 2308 can transmit the classifiers at once, either in one message or a series of messages near each other in time. In some configurations, the classifier factory 2308 can send the classifiers separated in time. For example, the classifier factory 2308 may generate and transmit classifiers. Later, with more BCG signals and training data available, the classifier factory 2308 may generate an updated classifier or a new classifier unlike one already created.

The classifier factory 2308 can transmit the classifiers to the bed controller 2304 of a bed that is different than the training data source 2302. For example, the training data source 2302 can be a first bed and the bed controller 2304 can be part of a second bed. The second bed can be different than the first bed and therefore the second bed can be used by different users than the first bed. In some implementations, the training data source 2302 can be a database and therefore the classifier factory 2308 can transmit the classifiers to a plurality of different beds that otherwise may not be associated with the training data source 2302.

The classifiers may be defined in one or more data structures. For example, the classifier factory 2308 can record a classifier in an executable or interpretable files such as a software library, executable file, or object file. The classifiers may be stored, used, or transmitted as a structured data object such as an extensible markup language (XML) document or a JavaScript object notation (JSON) object. In some examples, a classifier may be created in a binary or script format that the bed controller 2304 can run (e.g., execute or interpret). In some examples, a classifier may be created in a format that is not directly run, but in a format with data that allows the bed controller 2304 to construct the classifier according to the data.

In some implementations, the bed controller 2304 can also use the same BCG signals that were used for training in order to determine sleep states during run-time use in 2322. In some implementations, the bed controller 2304 can use different BCG signals to determine sleep states during run-time use. In some implementations, the bed controller 2304 can receive pressure readings of a user on the bed from one or more bed sensors. The bed sensors can include pressure sensors configured to detect changes in pressure in one or more air chambers of the bed. The bed sensors can also be configured to detect the user's body movements, heart rate, and/or breathing rate, as described throughout this disclosure.

For example, the bed controller 2304 can run one or more classifiers using data from the stream of BCG signals and/or pressure readings from the bed. The classifier can categorize this data into one of a plurality of states (e.g., awake, N1, N2, N3, REM). For example, the classifier may convert the data stream into a vector format described above. The classifier may then examine the vector to mathematically determine if the vector is more like training data labeled as one state or more like training data labeled as another state. Once this similarity is calculated, the categorizer can return a response indicating that state. The bed controller 2304 can also use the classifiers to determine a next sleep state for the user.

The bed controller 2304 can use more than one classifier. That is, the bed controller 2304 may have access to a plurality of classifiers that each function differently and/or use different training data to generate classifications. In such cases, classifier decisions can be treated as a vote and vote aggregation can be used to determine sleep state. If only one classifier is used, the vote of that classifier is the only vote and the vote is used as the sleep state. If there are multiple classifiers, the different classifiers can produce conflicting votes, and the bed controller can select a vote-winning sleep state.

Various vote-counting schemes are possible. In some cases, the bed controller 1094 can count the votes for each sleep state and the sleep state with the most votes is the determined sleep state. In some cases, the bed controller 2304 can use other vote-counting schemes. For example, votes from different classifiers may be weighed based on the classifiers historical accuracy. In such a scheme, classifiers that have been historically shown to be more accurate can be given greater weight while classifiers with lesser historical accuracy can be given less weight. This accuracy may be tracked on a population level or on a particular user level.

In some instances, votes may be cast by systems other than a machine-learning system, and those votes may be incorporated into the vote totals to impact the outcomes of the voting decision. For example, non-machine-learning pressure categorizing algorithms may cast votes based on, for example, comparisons with threshold values.

In some instances, the system may have different operational modes, and may tally votes differently depending on the mode. For example, when a bed is in the process of adjusting or when the adjustable foundation is moving or a portion of the bed is elevated, different vote strategies may be used. In some modes, some classifiers may be given greater weight or lesser weight or no weight as compared to some other modes. This may be useful, for example, when a classifier is shown to be accurate in one mode (e.g. with the bed flat) versus another mode (e.g., with the head of the bed elevated by the foundation).

In some cases, the bed controller 2304 can ensure that there is a user in bed before determining sleep state. For example, the bed controller can initially determine if the user is in the bed or if the bed is empty. This determination can be based on pressure signals that are received from pressure sensors on the bed. If the pressure signals indicate that no pressure is being added to a top surface of the bed, then the user most likely is not presently in the bed. If the user is determined to be in the bed (e.g., pressure signals indicate an amount of pressure on the top surface of the bed that exceeds some predetermined threshold value), the bed controller 2304 can determine if the user is asleep in the bed, and if so, in what sleep state.

In general, the process 2300 can be organized into a training time and an operating time. The training time can include actions that are generally used to create sleep state classifiers, while the operating time can include actions that are generally used to determine a sleep state with the classifiers. Depending on the configuration of the bed system, the actions of one or both of the times may be engaged or suspended. For example, when a user newly purchases a bed, the bed may have access to no pressure readings caused by the user on the bed. When the user begins using the bed for the first few nights, the bed system can collect those pressure readings and supply them to the cloud reporting service 2306 once a critical mass of readings have been collected (e.g. a certain number of readings, a certain number of nights, a certain number of expected entry and exit events based on different tests or heuristics).

The bed system may operate in the training time to update or expand the classifiers. The bed controller 2304 may continue actions of the training time after receipt of the classifiers. For example, the training data source 2302 may transmit BCG signals to the cloud reporting service 2306 on a regular basis, when computational resources are free, at user direction, etc. The classifier factory 2308 may generate and transmit new or updated classifiers, or may transmit messages indicating that one or more classifiers on the bed controller 2304 should be retired.

The bed system can use the same BCG signals from the training data source 2302 to operate in the training time and the operating time concurrently. For example, the bed system can use a stream of pressure readings to determine a sleep state based on sleep categorizers that are currently in use. In addition, the bed system can use the same pressure readings from the stream of pressure readings in the training time actions to improve the categorizers. In this way, a single stream of pressure readings may be used to both improve the function of the bed system and to determine sleep states of the user.

In some cases, a generic set of classifiers may be used instead of, or in conjunction with, personalized classifiers. For example, when a bed is newly purchased or reset to factory settings, the bed system may operate with generic or default sleep state classifiers that are created based on population-level, not individual, pressure readings. That is, generic classifiers may be created for use in a bed system before the bed system has had an opportunity to learn about the particular pressure readings associated with a particular user. These generic classifiers may be generated using machine learning techniques, such as those described in this document, on population-level training data. These generic classifiers may additionally or alternatively be generated using non-machine learning techniques. For example, a classifier may include a threshold value (e.g., pressure, pressure change over time, heart rate change over time, changes in physiological components of the pressure readings over time), and a pressure measure over that threshold may be used to determine one sleep state while pressure readings and/or physiological values under that threshold may be used to determine another sleep state.

While a particular number, order, and arrangement of elements are described here, other alternatives are possible. For example, while the generation of classifiers in 2316 is described as being performed on a classifier factory 2308, classifiers can be instead or additionally generated by the bed controller 2304 and/or the cloud reporting service 2306, possibly without reporting the BCG signals to other devices.

In some implementations, the bed system may accommodate two users. In such a case, the process 2300 can be adapted in one or more ways to accommodate two users. For example, for each user, the bed system may use two sets of classifiers (with or without some classifiers being simultaneously used in both sets.) For example, one set may be used when one side of the bed is occupied, and one set may be used when the other side of the bed is occupied. This may be useful, for example, when the presence or absence of the second user has an impact on BCG signals on the first user's side of the bed.

It will be understood that the system described in reference to FIG. 23 is applicable with many more beds and bed controllers. For example, BCG signals may be received from many training data sources, and training data can be synthesized from these many sources (which may or may not include beds), providing data about bed use by many users. The classifiers can then be distributed to some, none, or all of those training data sources or beds that provided training data. For example, some beds may receive a software updated with new classifiers. Or, as another example, the new classifiers may only be included on newly manufactured beds. Or as another example, each bed may receive classifiers that are particularly tailored to the users of that particular bed.

FIG. 24 is a flowchart of an example process 2400 that may be used to train a sleep-stage classifier. For clarity, the process 2400 is being described with reference to a particular set of components of a computing system. However, other system or systems can be used to perform the same or a similar process. For example, the process 2400 can be performed by a computing system having one or more processors and memory storing instructions that cause the one or more processors to perform the techniques described in reference to the process 2400. The computing system can be a cloud service. In some implementations, the computing system can also be a bed controller. The computing system can be any other system or systems.

Referring to the process 2400, the computing system can receive input in 2402. The computing system can, for example, receive cardiac data defining at least inter-beat interval (IBI) sequences. This input can also include other BCG signals including but not limited to breathing rate and/or body movements. This input can be received from a training data source (e.g., refer to FIG. 23). The input can also be received from sensors of one or more bed systems. The input can include tagging data that otherwise defines tags of sleep-states for the IBI sequences. Moreover, PPG, ECG, and/or BCG signals can be provided as input and tagged, since these signals can produce sequences of IBIs during sleep. The IBI unit (seconds, or more commonly milliseconds) is time, which may the same across platforms and therefore would not require calibration when received as input from a variety of different devices and/or training data sources.

In 2404, the computing system can train a convolutional neural network (CNN) using the input. By training the CNN, the computing system can generate a sleep-state classifier using the cardiac data and the tagging data. For example, the computing system can extract the IBI sequences from the cardiac data. The computing system can then train the CNN using as input the cardiac data and the tagging data. The computing system can train the CNN to map or otherwise correlate sleep states defined by the tagging data with different segments of the IBI sequences.

The computing system can generate intermediate data in 2406. The intermediate data can be output generated by the trained CNN. The computing system can also filter this intermediate data.

The computing system can apply a trained long-short term memory (LSTM) network to the generated intermediate data in 2408. In other words, the computing system can iteratively train a recurrent neural network (RNN) configured to produce state data as output. The iterative training of the RNN can use i) the intermediate data as an initial input and ii) the intermediate data and a previous state data as subsequent input. The RNN can further include at least one other input node for the intermediate data.

Thus, the computing system can iteratively apply the LSTM (e.g., an example RNN) to the intermediate data in 2408 for every sleep state of a user. The LSTM can include at least one feedback connection that connects an output mode of the recurrent neural network to an input node of the same recurrent neural network. This configuration can be advantageous to ensure that the most recently generated data can be used as input in order to train the CNN to more accurately determine sleep states.

The computing system can then generate output in 2410. The output can include sleep states mapped onto different IBI sequences and/or other cardiac data. In other words, the output can include one or more classifiers. Each of the classifiers can correlate sleep states with different IBI sequences. The output can then be used by one or more bed controllers and/or bed systems to determine real-time sleep states of users based on pressure signals that are sensed by sensors of the bed system.

FIG. 25 is a flowchart of an example process 2500 for determining user presence in a bed. The process 2500 may be performed by any technologically appropriate system, including but not limited to the systems described throughout this disclosure.

Referring to the process 2500 in FIG. 25, a computer system, such as a controller of a bed system and/or the computer system 1906 described in reference to FIG. 19, can determine that a mattress is unoccupied in 2502. For example, the computer system may determine that the pressure of the mattress is constant for a period of time, or a previously performed presence test may return a negative result.

A stream of pump pressure readings are received from a mattress pump at the computer system in 2504. The pump pressure readings record the air pressure of the mattress. For example, the pump of a mattress may include a sensor that detects the air pressure of the mattress. This pump may be configured to report these readings in a stream to a computer system. The stream of data may include, but is not limited to, a reporting of readings on a regular schedule, reporting in response to requests for the reading, and other schemes.

The computer system identifies an increase in pump pressure readings within a time window in 2506. For example, the computer system may identify that, within a period of time or a number of readings, the value of the readings increases more than a threshold value. This may represent, for example, a sharp increase in air pressure within the mattress.

After identifying the increase in pump pressure readings within the time window and for each received pump pressure readings, the computer system determines that a difference is less than a threshold value in 2508. For example, until a test generating a difference value returns a difference smaller than a threshold value, the test may be repeated for each received pump pressure value. The test may also be performed on a different sampling frequency (e.g., every other or every third pump pressure value received).

The computer system calculates a trailing average pressure that represents the average of the N most recent pump pressures readings in the stream of pump pressure readings in 2510. For example, if N=7, the computer system may average the most current pump pressure reading with the six preceding that have been stored in memory.

The computer system determines the difference between the received pump pressure reading and the trailing average pressure in 2512. For example, the computer system may subtract the trailing average from the received pump pressure reading to determine the difference. That difference is then compared to a threshold value that may be, for example, pre-determined or calculated in response to changing parameters.

Responsive to the computer system determining that the difference is less than the threshold value, presence is determined in 2514. For example, a value may be stored in computer readable memory to record the presence, a computing event may be raised, and/or a peripheral device may be engaged. Refer to FIGS. 19-22 for discussion about how bed presence detection can be used to detect and/or prevent sleepwalking events.

FIG. 26 is a flowchart of another example process 2600 for determining user presence in a bed. The process 2600 may be performed by any technologically appropriate system, including but not limited to the systems described throughout this disclosure.

A computer system, such as the computer system 1906 described in FIG. 19 and/or a controller of a bed system, determines that a mattress is unoccupied in 2602. For example, the computer system may determine that the pressure of the mattress is constant for a period of time, or a previously performed presence test may return a negative result.

An empty pump pressure reading is received from a mattress pump and at a computer system in 2604. The empty pump pressure reading records the air pressure of the mattress when the mattress is not subject to pressure from a person. For example, the computer system may record the constant pressure as the empty pump pressure reading.

A stream of pump pressure readings are received from a mattress pump at the computer system in 2606. The pump pressure readings record the air pressure of the mattress when the mattress is subject to pressure from an external body.

For each received pump pressure reading, the computer system performs a test in 2608. For example, until a test comparing pressure readings to a threshold value returns true, the test may be repeated for each received pump pressure value. The test may also be performed on a different sampling frequency (e.g., every other or every third pump pressure value received).

The computer system calculates a test value that includes the most recent pump pressure reading in the stream of pump pressure readings in 2610. For example, the test value may be the most recent pump pressure value, a weighted or weighted average, and/or a smoothed value.

The computer system determines that the test value is greater than a threshold value that is greater than the empty pump pressure reading in 2612. For example, if the test value is 2382 and the threshold value is 1849, the computer system determines that the test value is greater.

Responsive to the computer system determining that the difference is less than the threshold value, presence is determined in 2614. For example, a value may be stored in computer readable memory to record the presence, a computing event may be raised, and/or a peripheral device may be engaged. Refer to FIGS. 19-22 for discussion about how bed presence detection can be used to detect and/or prevent sleepwalking events.

In some implementations, a pressure transducer of a bed system can be used to detect a user's presence on the bed, e.g., via a gross pressure change determination and/or via one or more of a respiration rate signal, heart rate signal, and/or other biometric signals. For example, a simple pressure detection process can identify an increase in pressure as an indication that the user is present in the bed. As another example, user presence can be determined based on detected pressure increases above a specified threshold (so as to indicate that a person or other object above a certain weight is positioned on the bed). As yet another example, an increase in pressure can be identified in combination with detected slight, rhythmic fluctuations in pressure, which can correspond to the user being present on the bed. The presence of rhythmic fluctuations can be identified as being caused by respiration or heart rhythm (or both) of the user. The detection of respiration or a heartbeat can also distinguish between the user being present on the bed and another object (e.g., a suit case) being placed upon the bed.

In some implementations, the user's presence on the bed can be determined using a variety of signals received from sensors of the bed. For example, user bed presence can be determined using temperature signals detected by temperature sensors on a top surface of the bed. A model can be trained to correlate spikes in sensed temperature with bed entrance events and dips in sensed temperature with bed exit events. The computer system can also determine bed presence based on a combination of pressure and temperature signals. Moreover, bed presence detection techniques can be performed using any other combination of sensors at the bed. If, for example, the bed includes load cells, signals detected by the load cells can be used with the disclosed techniques to detect bed presence. If the bed includes audio sensors, as another example, signals detected by the audio sensors can be used with the disclosed techniques to detect bed presence. Moreover, as mentioned above, any combination of such signals can be used to detect bed presence. As non-limiting examples, bed presence detection can be performed with the disclosed techniques using a combination of (i) pressure and temperature signals from pressure and temperature sensors of the bed, (ii) temperature and load cell signals from temperature sensors and load sensors of the bed, (iii) pressure, temperature, and load cell signals, (iv) pressure and load cell signals, (v) audio and pressure signals, (vi) audio and temperature signals, (vii) audio and load cell signals, (viii) audio, pressure, temperature, and load cell signals, and/or any other combination thereof

What is claimed is:

1. A system for detecting sleepwalking events of a user in a bed, the system comprising:
at least one sensor; and
a computer system in communication with the at least one sensor, the computer system configured to:
receive sensor data from the at least one sensor during a sleep session of a user of a bed;
provide, as input to a sleep state classifier, a first portion of the sensor data, wherein the sleep state classifier uses a machine-learning model to determine the user's sleep states during the sleep session;
receive, as output from the sleep state classifier, a sleep state classification for the user;
provide, as input to a bed exit detection classifier, a second portion of the sensor data, wherein the bed exit detection classifier uses a machine-learning model to determine when the user exits the bed during the sleep session;
receive, as output from the bed exit detection classifier, a bed exit detection classification for the user;
determine whether (i) the sleep state classification for the user satisfies a first threshold condition and (ii) the bed exit detection classification for the user satisfies a second threshold condition;
identify, based on a determination that the first and the second threshold conditions are satisfied, a sleepwalking event for the user; and
generate output based on identification of the sleepwalking event.

2. The system of claim 1, wherein the first threshold condition is a N3 sleep state.

3. The system of claim 1, wherein the second threshold condition is detection of at least one bed exit event.

4. The system of claim 1, wherein the computer system is further configured to:
identify, based on (i) the determination that the first and the second threshold conditions are satisfied and (ii) a total time from a start of the sleep session is within a threshold time range since sleep onset, wherein the threshold time range is associated with prior sleep session time ranges associated the user, the sleepwalking event for the user.

5. The system of claim 4, wherein the threshold range is 1 to 3 hours.

6. The system of claim 4, wherein the threshold range is 1 to 2 sleep cycles.

7. The system of claim 4, wherein generating the output comprises storing the sleep state classification, the bed exit detection classification, and the total time in a data store.

8. The system of claim 1, wherein generating the output comprises storing the identification of the sleepwalking event in a data store.

9. The system of claim 8, wherein the identification of the sleepwalking event includes information about a time during the user's sleep session when the sleepwalking event was identified.

10. The system of claim 1, wherein generating the output comprises generating a notification indicating that the sleepwalking event was identified during the user's sleep session.

11. The system of claim 10, wherein the computer system is further configured to transmit the notification to a user device of the user for presentation in a graphical user interface (GUI) display when the user wakes up from the sleep session.

12. The system of claim 10, wherein the computer system is further configured to transmit the notification to a user device of a healthcare provider associated with the user, wherein the notification is a machine-instruction to engage an automated device.

13. The system of claim 1, wherein the computer system is further configured to generate, based on a determination that the first and the second threshold conditions are satisfied, a probability of the sleepwalking event for the user.

14. The system of claim 13, wherein the probability of the sleepwalking event indicates a likelihood that the user will experience the sleepwalking event within a threshold amount of time from a current time during the sleep session.

15. The system of claim 14, wherein the threshold amount of time is 1 to 15 minutes.

16. The system of claim 13, wherein the computer system is further configured to generate an intervention to prevent onset of the sleepwalking event for the user based on the probability of the sleepwalking event for the user, wherein generating the intervention comprises transmitting instructions to a controller of the bed that, when executed by the controller, cause the controller to actuate a heating element in the bed to increase a temperature of a microclimate of the bed by a threshold amount.

17. The system of claim 16, wherein generating the intervention comprises transmitting instructions to the controller of the bed that, when executed by the controller, cause the controller to lower an adjustable foundation of the bed to a threshold height.

18. The system of claim 1, wherein:
the bed exit detection classification indicates a time at which bed presence of the user is detected, and
the bed exit detection classification for the user satisfies the second threshold condition based on the time at which the bed presence of the user is detected corresponding to historic data of detected sleepwalking events for the user.

19. A method for detecting sleepwalking events of a user in a bed, the method comprising:
receiving, by a computer system, sensor data from at least one sensor during a sleep session of a user of a bed;
providing, by the computer system and as input to a sleep state classifier, a first portion of the sensor data, wherein the sleep state classifier uses a machine-learning model to determine the user's sleep states during the sleep session;
receiving, by the computer system and as output from the sleep state classifier, a sleep state classification for the user;
providing, by the computer system and as input to a bed exit detection classifier, a second portion of the sensor data, wherein the bed exit detection classifier uses a machine-learning model to determine when the user exits the bed during the sleep session;
receiving, by the computer system and as output from the bed exit detection classifier, a bed exit detection classification for the user;
determining, by the computer system, whether (i) the sleep state classification for the user satisfies a first threshold condition and (ii) the bed exit detection classification for the user satisfies a second threshold condition;
identifying, by the computer system and based on a determination that the first and the second threshold conditions are satisfied, a sleepwalking event for the user; and
generating, by the computer system, output based on identification of the sleepwalking event.

20. The method of claim 19, wherein generating the output comprises generating a notification indicating that the sleepwalking event was identified during the user's sleep session.
